(19) United States
(12) Patent Application Publication
(10) Pub. No.: US 2023/0049700 A1
(43) Pub. Date: Feb. 16, 2023

(54) AUTOANTIBODIES AS BIOMARKERS FOR AUTOIMMUNE POLYGLANDULAR SYNDROME TYPE 1

(71) Applicants: Chan Zuckerberg Biohub, Inc., San Francisco, CA (US); The Regents of the University of California, Oakland, CA (US)

(72) Inventors: Caleigh Mandel-Brehm, Oakland, CA (US); Sara Vazquez, Oakland, CA (US); Mark S. Anderson, Oakland, CA (US); Joseph L. DeRisi, San Francisco, CA (US)

(21) Appl. No.: 17/788,552

(22) PCT Filed: Dec. 28, 2020
(86) PCT No.: PCT/US2020/067190

§ 371 Date: Jun. 23, 2022

Related U.S. Application Data
(60) Provisional application No. 62/955,220, filed on Dec. 30, 2019.

(51) Int. Cl. G01N 33/564; G01N 33/68; G01N 33/543; C07K 16/00

(52) U.S. Cl. CPC G01N 33/564; G01N 33/6893; G01N 33/543; C07K 16/00; G01N 2800/06; G01N 2800/18; G01N 2800/50

(57) ABSTRACT

This disclosure describes methods and compositions for detecting the presence of autoantibodies associated with autoimmune polyglandualar syndrome 1 (APS1) in a biological sample. In particular, detection of APS1-specific autoantibodies that specifically bind to certain APS1 associated autoantigens in such methods is described. Also provided are methods of treating subjects with APS1 or at risk of developing APS1 associated conditions. Also provided are devices and kits useful for the diagnosis and prognostic assessment of subjects having APS1 and for assessing subject risk at developing particular APS1-associated conditions.

### CROSS-REFERENCES TO RELATED APPLICATIONS

This application claims the benefit of and priority to U.S. Provisional Application No. 62/955,220 filed on Dec. 30, 2019, which is hereby incorporated by reference in its entirety.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named seqlist 103182-1216825.txt, created on Dec. 18, 2020, and having a size of 184 kb and is filed concurrently with the specification. The sequence listing contained in this ASCII formatted document is part of the specification and is herein incorporated by reference in its entirety.

### BACKGROUND

The identification of autoantigens remains a critical challenge for understanding and treating autoimmune diseases. Autoimmune polyendocrine syndrome type 1 (APS1) is a disease caused by monogenic mutations in the AIRE gene that result in defects in AIRE-dependent T cell education in the thymus. As a result, patients develop autoimmunity to multiple organs, including endocrine organs, skin, and lung. Although the majority of APS1 autoimmune manifestations are thought to be primarily driven by autoreactive T cells, patients also possess autoreactive B cells and corresponding high-affinity autoantibody responses. Some of these known autoantibody specificities for APS1 exhibit strong, clinically useful associations with their respective organ-specific diseases. For example, in autoimmune lung disease and adrenal insufficiency, there are clearly defined antigenic targets that provide predictive and diagnostic clinical benefits. However, for many of the other clinical manifestations of this syndrome, the underlying cell-type specific candidate antigens remain unknown.

### BRIEF SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim.

Described in this disclosure are methods, devices, and kits useful for the diagnosis and prognostic assessment of subjects having autoimmune polyendocrine syndrome type 1 (APS1), for assessing subject risk at developing particular APS1 associated conditions, and method of treating subjects with APS1.

In one aspect, provided is a method of detecting the presence of an autoantibody associated with autoimmune polyglandular syndrome type 1 (APS1) in a biological sample from a subject suspected to have APS1, or a subject diagnosed with APS1, comprising the steps of:

(a) contacting the biological sample with one or more antigenic polypeptides listed in Table 1, or a fragment of one or more of the antigenic polypeptides listed in Table 1; and

(b) detecting the presence of binding of the antigenic polypeptide or fragment thereof to an APS1 associated autoantibody in the biological sample.

In some instances, the subject has been determined to have a loss of function mutation in the AIRE gene.

In some instances, the subject has one or more of nail dystrophy, hypoparathyroidism, keratoconjunctivitis, chronic mucocutaneous candidiasis, intestinal dysfunction, autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis.

In some instances, the subject has at least one of intestinal dysfunction or primary ovarian insufficiency.

In some instances, the presence of the binding of the antigenic polypeptide or fragment thereof in the biological sample to one or more of said RFX6 autoantibody, KHDC3L autoantibody, ACPT autoantibody, or PLIN1 autoantibody indicates that the subject has APS1.

In some instances, the subject has diarrheal-type intestinal dysfunction, and the RFX6 antigenic polypeptide or fragment thereof binds to said RFX6 autoantibody in the biological sample. In some instances, the subject has premature ovarian failure, and the KHDC3L antigenic polypeptide or fragment thereof binds to said KHDC3L autoantibody in the biological sample. In some instances, the subject has dental enamel hypoplasia, and the ACPT antigenic polypeptide or fragment thereof binds to said ACPT autoantibody in the biological sample. In some instances, the subject has lipodystrophy, and wherein the PLIN1 antigenic polypeptide or fragment thereof binds to said PLIN1 autoantibody in the biological sample.

In another aspect, provided is a method of identifying a subject having a loss of function mutation in the AIRE gene that is at risk of developing diarrheal-type intestinal dysfunction, the method comprising:

(a) contacting a biological sample from the subject with a RFX6 antigenic polypeptide or fragment thereof; and

(b) detecting the presence of binding of the RFX6 antigenic polypeptide or fragment thereof to a RFX6 autoantibody in the biological sample.

In some instances, the subject is at risk of developing diarrheal-type intestinal dysfunction, and the subject has one or more of nail dystrophy, hypoparathyroidism, keratoconjunctivitis, chronic mucocutaneous candidiasis, autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis.

In another aspect, provided is a method of identifying a subject having a loss of function mutation in the AIRE gene that is at risk of developing primary ovarian insufficiency, the method comprising:

(a) contacting a biological sample from the subject with a KHDC3L antigenic polypeptide or fragment thereof; and

(b) detecting the presence of binding of the KHDC3L antigenic polypeptide or fragment thereof to a KHDC3L autoantibody in the biological sample.

In some instances, the subject is at risk of developing primary ovarian insufficiency, and the subject has one or more of nail dystrophy, hypoparathyroidism, keratoconjunctivitis, chronic mucocutaneous candidiasis, diarrheal-type intestinal dysfunction, autoimmune hepatitis, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis.

In another aspect, provided is a method of identifying a subject having a loss of function mutation in the AIRE gene that is at risk of developing dental enamel hypoplasia, the method comprising:

(a) contacting a biological sample from the subject with an ACPT antigenic polypeptide or fragment thereof; and

(b) detecting the presence of binding of the ACPT antigenic polypeptide or fragment thereof to an ACPT autoantibody in the biological sample.

In some instances, the subject is at risk of developing dental enamel hypoplasia, and the subject has one or more of nail dystrophy, hypoparathyroidism, keratoconjunctivitis, chronic mucocutaneous candidiasis, diarrheal-type intestinal dysfunction, autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, testicular failure, tubulointerstital nephritis, hypopituitarism, vitiligo, gastritis, urticarial eruption, alopecia, asplenia, lipodystrophy, or pneumonitis.

In another aspect, provided is a method of identifying a subject having a loss of function mutation in the AIRE gene that is at risk of developing lipodystrophy, the method comprising:

(a) contacting a biological sample from the subject with a PLIN1 antigenic polypeptide or fragment thereof; and

(b) detecting the presence of binding of the PLIN1 antigenic polypeptide or fragment thereof to a PLIN1 autoantibody in the biological sample.

In some instances, the subject is at risk of developing lipodystrophy, and the subject has one or more of nail dystrophy, hypoparathyroidism, keratoconjunctivitis, chronic mucocutaneous candidiasis, diarrheal-type intestinal dysfunction, autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, or pneumonitis.

In some instances, step (b) of detecting the presence of binding of an antigenic polypeptide or fragment thereof to an autoantibody in a biological sample in the methods provided herein is performed by at least one of immunoprecipitation, microarray analysis, enzyme-linked immunosorbent assay (ELISA), or Western blot analysis.

In some instances, the biological sample is serum, plasma, cerebrospinal fluid, blood, or urine.

In some instances, the antigenic polypeptide or fragment thereof is heterologously-expressed on the surface of a cell, a phage or a virus.

In some instances, the antigenic polypeptide or fragment thereof is expressed in a phage display or eukaryotic cell display library.

In some instances, the antigenic polypeptide or fragment thereof is an isolated, purified antigenic polypeptide or fragment thereof.

In some instances, the antigenic polypeptide or fragment thereof is an isolated, purified antigenic polypeptide or fragment thereof that is immobilized on a solid carrier.

In some instances, the RFX6 antigenic polypeptide comprises the sequences of SEQ ID NO:24. In some instances, the RFX6 antigenic polypeptide or fragment thereof comprises one or more of the sequences of SEQ ID NOs: 31-37. In some instances, the KHDC3L antigenic polypeptide comprises the sequence of SEQ ID NO: 13. In some instances, the KHDC3L antigenic polypeptide or fragment thereof comprises one or more of the sequences of SEQ ID NOs: 38-40. In some instances, the ACPT antigenic polypeptide comprises the sequence of SEQ ID NO: 29. In some instances, the ACPT antigenic polypeptide or fragment thereof comprises the sequence of SEQ ID NO: 41. In some instances, the PLIN1 antigenic polypeptide comprises the sequence of SEQ ID NO: 30. In some instances, the PLIN1 antigenic polypeptide or fragment thereof comprises one or more of the sequences of SEQ ID NOs: 42-46.

In some instances, the presence of the binding of the RFX6 antigenic polypeptide or fragment thereof to the RFX6 autoantibody in the biological sample indicates that the subject is at risk of developing diarrheal-type intestinal dysfunction. In some instances, the presence of the binding of the KHDC3L antigenic polypeptide or fragment thereof to the KHDC3L autoantibody in the biological sample indicates that the subject is at risk of developing primary ovarian insufficiency. In some instances, the presence of the binding of the ACPT antigenic polypeptide or fragment thereof to the ACPT autoantibody in the biological sample indicates that the subject is at risk of developing dental enamel hypoplasia. In some instances, presence of the binding of the PLIN1 antigenic polypeptide or fragment thereof to the PLIN1 autoantibody in the biological sample indicates that the subject is at risk of developing lipodystrophy.

In another aspect, provided herein are devices, kits and panels useful for diagnosis and prognostic assessment of subjects having autoimmune polyendocrine syndrome type 1 (APS1), and for assessing subjects at risk of developing particular APS1 associated conditions. In some aspect, provided in this disclosure are kits and panels containing one or more RFX6 polypeptides or antigenic fragments thereof to which RFX6 autoantibodies can specifically bind. In some aspect, provided in this disclosure are kits and panels containing one or more KHDC3L polypeptides or antigenic fragments thereof to which KHDC3L autoantibodies can specifically bind. In some aspect, provided in this disclosure are kits and panels containing one or more ACPT polypeptides or antigenic fragments thereof to which ACPT autoantibodies can specifically bind. In some aspect, provided in this disclosure are kits and panels containing one or more PLIN1 polypeptides or antigenic fragments thereof to which PLIN1 autoantibodies can specifically bind. The polypeptide used in the kits and panels is preferably designed such that it is immunogenic, particularly that it binds to PLIN1 autoantibodies from subjects.

In another aspect, provided is a method of treating a subject having autoimmune polyglandular syndrome type 1 (APS1), comprising the steps of:

(a) detecting the presence an APS1 associated autoantibody that binds specifically to an antigenic polypeptide or fragment thereof listed in Table 1 in a biological sample from a subject, where detecting one or both of the autoantibodies in the biological sample indicates that the subject has APS1; and

(b) administering to the subject an immunosuppressive therapy.

In another aspect, provided is a method of treating a subject having APS1, comprising administering an immunosuppressive therapy to a subject expressing an APS1 associated autoantibody that binds specifically to an antigenic polypeptide or fragment thereof listed in Table 1.

In some instances, the subject being treated has one or more of nail dystrophy, hypoparathyroidism, keratoconjunctivitis, chronic mucocutaneous candidiasis, intestinal dysfunction, autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis.

In some instances, the subject being treated has at least one of intestinal dysfunction, primary ovarian insufficiency, dental enamel hypoplasia, or lipodystrophy.

In some instances, the subject being treated has diarrheal-type intestinal dysfunction, and the presence or absence of a RFX6 autoantibody is detected in the biological sample. In some instances, the subject has primary ovarian insufficiency, and the presence or absence of a KHDC3L autoantibody is detected in the biological sample. In some instances, the subject has dental enamel hypoplasia, and the presence or absence of an ACPT autoantibody is detected in the biological sample. In some instances, the subject has lipodystrophy, and wherein the presence or absence of a PLIN1 autoantibody is detected in the biological sample.

In another aspect, provided is a method of treating a subject that is at risk of developing one or more of diarrheal-type intestinal dysfunction, primary ovarian insufficiency, dental enamel hypoplasia, or lipodystrophy comprising the steps of:

(a) detecting the presence or absence of one or more of a RFX6 autoantibody, a KHDC3L autoantibody, an ACPT autoantibody, or a PLIN1 antibody in a biological sample from a subject, where detecting one or both of the autoantibodies in the biological sample indicates that the subject is at risk of developing one or more of diarrheal-type intestinal dysfunction, primary ovarian insufficiency, dental enamel hypoplasia, or lipodystrophy; and

(b) administering to the subject an immunosuppressive therapy.

In another aspect, provided is a method of treating a subject that is at risk of developing one or more of diarrheal-type intestinal dysfunction, primary ovarian insufficiency, dental enamel hypoplasia, or lipodystrophy comprising, comprising administering an immunosuppressive therapy to a subject expressing a RFX6 autoantibody, a KHDC3L autoantibody, an ACPT autoantibody, and/or a PLIN1 antibody.

In some instances, the subject being treated has been determined to have a loss of function mutation in the AIRE gene.

In some instances, the subject being treated has one or more of nail dystrophy, hypoparathyroidism, keratoconjunctivitis, chronic mucocutaneous candidiasis, intestinal dysfunction, autoimmune hepatitis, hypertension, hypothyroidism, primary ovarian insufficiency, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis.

In some instances, the immunosuppressive therapy comprises at least one of an immunosuppressant drug, intravenous immunoglobulin administration, plasma exchange plasmapheresis, immunoadsorption, or oral administration of the antigenic polypeptide or immunogenic fragments thereof.

In some instances, in the provided methods of treatment, the subject being treated is also administered:

(i) a therapy for diarrheal-type intestinal dysfunction if a RFX6 autoantibody is detected in the biological sample;

(ii) a therapy for primary ovarian insufficiency if a KHDC3L autoantibody is detected in the biological sample;

(iii) a therapy for dental enamel hypoplasia if an ACPT autoantibody is detected in the biological sample; and/or

(iv) a therapy for lipodystrophy if a PLIN1 autoantibody is detected in the biological sample.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows radioligand binding assay (RLBA) orthogonal validation of literature-reported antigens CYP11A1, SOX10, and NLRP5 within the expanded cohort of APS1 (n=67) and healthy controls (n=61) according to aspects of this disclosure. For each antigen, p-value was calculated across all samples using a Mann-Whitney U test.

FIG. 2 shows scatterplots of PhIP-seq enrichment values (log 10) over mock-IP as compared to RLBA antibody index (1=commercial antibody signal) for literature-reported antigens CYP11A1, SOX10, and NLRP5 according to aspects of this disclosure. Pearson correlation coefficient r values are shown on each plot.

FIG. 3 shows a plot of the identified known and novel PhIP-seq autoantigens that were shared across the highest number of individual APS1 sera (left panel) according to aspects of this disclosure. ASMT and PDX1 were positive hits in 3 and 2 sera, respectively, but are known to be highly tissue specific (right panel). Antigens marked with asterisks (*) were chosen for validation in whole protein binding assay (shown in FIG. 4) according to aspects of this disclosure.

FIG. 4 shows validation of novel PhIP-seq antigens as selected from those identified in FIG. 3 (marked with *) by radiolabeled binding assay according to aspects of this disclosure. Analysis was performed with discovery cohort sera (black, nAPS1=39), validation cohort sera (light grey, nAPS1=28), and healthy control sera (nHC=61). For each antigen, p-value was calculated across all samples using a Mann-Whitney U test.

FIG. 5 shows a heatmap of p-values (Kolmogorov-Smirnov testing) for differences in gene enrichments for patients with versus without each clinical phenotype according to aspects of this disclosure. Significant p-values in the negative direction (where mean PhIP-Seq enrichment is higher in patients without disease) are masked (colored >0.05). Disease-antigen correlations marked with “A”, “B”, and “C” are detailed in FIG. 6 (with corresponding markings) and Example 6, below. The disease-antigen correlation marked with a black arrowhead is detailed in FIG. 9 and FIG. 10 and Example 10, below. Genes are listed on the left; Disease manifestations are listed across the top. ND, nail dystrophy. HP, hypoparathyroidism. KC, keratoconjunctivitis. CMC, chronic mucocutaneous candidiasis. ID (D), Intestinal dysfunction (diarrheal-type). AIH, autoimmune hepatitis. POI, primary ovarian insufficiency. HTN, hypertension. HT, hypothyroidism. B12 def, B12 (vitamin) deficiency. DM, diabetes mellitus. SS, Sjogren's-like syndrome. Pneum, Pneumonitis. GH def, Growth hormone deficiency. AI, Adrenal Insufficiency. EH, (dental) enamel hypoplasia.

FIG. 6 shows heatmaps illustrating the APS1 disease-antigen correlations as identified in FIG. 5 according to aspects of this disclosure. For each antigen, p-value was calculated by Kolmogorov-Smirnov test. Panels “A”, “B”, and “C” correspond to the disease-antigen correlations identified in FIG. 5. Top panel marked “A” shows a comparison of anti-CYP11A1 PhIP-seq enrichments between APS1 patients with and without adrenal insufficiency (p=0.0080). The middle panel marked “B” shows a comparison of anti-KHDC3L PhIP-seq enrichments between APS1 patients with and without ovarian insufficiency (p=0.0016). The bottom panel marked “C” shows a comparison of anti-SOX10 PhIP-seq enrichments between APS1 patients with and without Vitiligo (p=0.0002).

FIG. 7 shows scatterplots illustrating the distribution of RLBA antibody indices for both KHDC3L (left panel) and NLRP5 (right panel) in APS1 patients divided into male and female patients according to aspects of this disclosure.

FIG. 8 shows a plot of RLBA antibody indices for both KHDC3L and NLRP5 for female APS1 patients by age according to aspects of this disclosure. Anti-KHDC3L antibodies were found to have 100% sensitivity for primary ovarian insufficiency (POI). Note that many of the patients with anti-KHDC3L antibodies but without POI are younger and therefore cannot be fully evaluated for ovarian insufficiency.

FIG. 9 shows strip plots illustrating RLBA antibody index for RFX6 in patients without and with intestinal dysfunction (“ID −” and “ID +”, respectively) according to aspects of this disclosure. Patient data points are grey or black circles, with grey circles (“RLBA+”) indicating patients that fall above 6 standard deviations of the mean over healthy control RLBA antibody index. p=0.006 by Mann-Whitney U test.

FIG. 10 shows violin plots comparing the anti-RFX6 antibody index amongst patients with the diarrheal subtype of intestinal dysfunction (ID), patients with the constipation subtype of ID, and patients without ID according to aspects of this disclosure. Patients with the diarrhea subtype were found to have a higher frequency of anti-RFX6 antibody positivity as compared to patients with the constipation subtype (p=0.0028 by Mann-Whitney U test) or no ID (p=0.0015 by Mann-Whitney U test) according to aspects of this disclosure.

FIG. 11 shows clustered disease correlations in the APS1 patient cohort according to aspects of this disclosure. Association between the APS1 associated conditions (i.e. phenotypes) was assessed using Spearnman's rank correlation coefficient. The associated heat map is not shown. n=67. Disease condition abbreviations are the same as set forth for FIG. 5 plus Urt, Urticarial eruption and Pneum, pneumonitis.

FIG. 12 shows PhIP-Seq analysis results illustrating the discovery and validation of autoantibodies to Perilipin-1 in Aire −/− mouse sera with known idiopathic antibodies according to aspects of this disclosure. Individualized PhIP-Seq analysis show reads per 100,000 for PLIN1 protein from Aire +/+ mice (n=3) and Aire −/− mice (n=4).

FIG. 13 shows gel images illustrating immunoblotting results that validate antibody reactivity to PLIN1 according to aspects of this disclosure. Whole cell lysates generated from 293T cells expressing full-length mouse PLIN1 were incubated with sera from AIRE (n=4) or WT (n=3) mice. Antibodies were immunoprecipitated using AG beads and IP elutions were subject to SDS-PAGE immunoblotting. IP elutions were immunostained with either anti-Flag IgG to identify positive ZSCAN1 signal, or anti-mouse IgG to show qualitative capture of IgG from sera.

FIG. 14 shows bar graphs illustrating relative expression of Plin1 in Aire+/+ versus Aire−/− mTECS according to aspects of this disclosure. Dataset were re-analyzed from Sansom et al 2014.

FIG. 15 shows a plot illustrating radioligand binding assay results that indicate autoantibodies to PLIN1 in sera from a patient with APS1 involving acquired generalized lipodystrophy (AGL) according to aspects of this disclosure. Human PLIN1 protein (in vitro T7 transcribed) radiolabeled with ³⁵S-methionine was used. Radiolabeled PLIN1 was incubated with sera from healthy controls (HC, n=54) or from APS1 patients with or without Lipodystrophy (APS1+/−LD, n=68/1). Dotted line indicates mean+3 std. dev. of healthy controls.

### DETAILED DESCRIPTION

As used in the disclosure and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations, and methodologies which are described in the publication and which might be used in connection with the presently described invention.

Identification of the specificity of autoantibodies in autoimmune diseases is important for understanding underlying disease pathogenesis and for identifying those at risk for disease. Despite the long-known association of autoantibodies with specific autoimmune diseases in both monogenic and sporadic autoimmunity, many autoantibody specificities have yet to be fully determined. Challenges in antigen identification include the weak affinity of some autoantibodies for their target antigen, as well as rare or low expression pattern of the target antigen. Autoimmune polyendocrine syndrome type 1 (APS1) is a defined monogenic autoimmune syndrome with a broad spectrum of high affinity autoantibodies. In this invention, PhIP-seq interrogation of APS1 was used to yield clinically meaningful targets, consistent with previously described APS1 autoantibody specificities that exhibit strong, clinically useful associations with their respective organ-specific diseases.

The methods provided herein are useful for the evaluation of subjects who present with one or more clinical symptoms associated with autoimmune polyendocrine syndrome type 1 (APS1) for novel autoantibodies to assist in clinical diagnosis and appropriate therapeutic intervention. In some instances, the methods may identify a subject that does not have a particular condition associated with APS1 but who is as at risk of developing such condition. Such patients can then be monitored for development of such conditions so that treatment can be administered prophylactically and/or promptly.

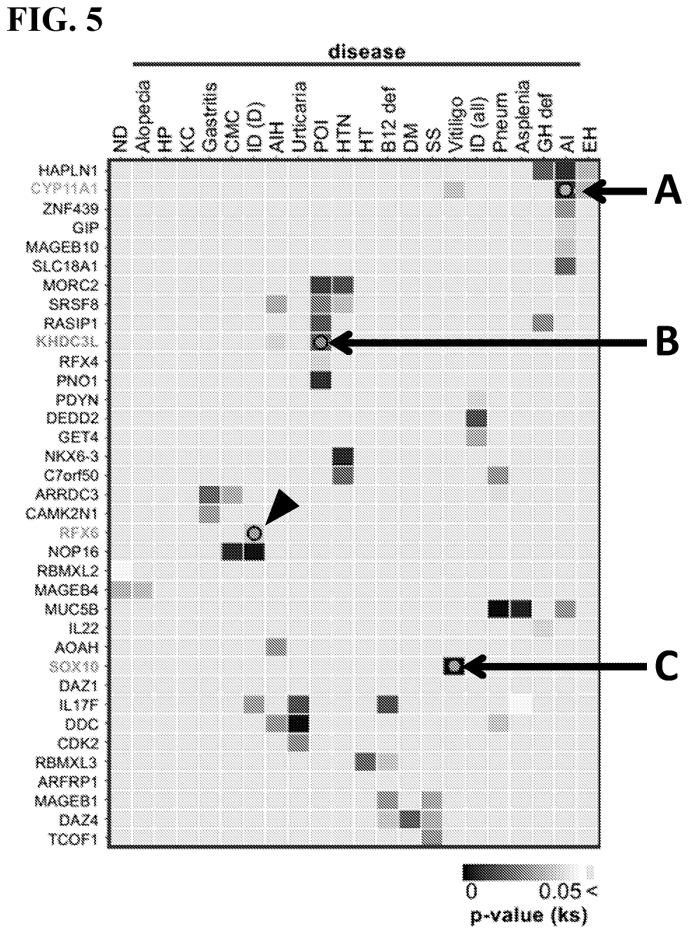

As described in this disclosure, autoantibody markers have been identified in patients with APS1 that bind to previously unknown APS1 associated autoantigens. These novel APS1 autoantigens exhibit significant tissue-restriction, such as expression in enteroendocrine cells, pineal gland, and dental enamel depending on the antigen, and are targeted in organ-restricted autoimmunity. The novel autoantigens identified in this disclosure are listed in Table 1. Specifically, some of the new APS1 associated autoantigens provided in this disclosure are ACPT, AOAH, ARFRP1, ARRDC3, C7orf50, CAMK2N1, CDK2, DAZ1, DAZ4, DEDD2, GET4, GIP, HAPLN1, KHDC3L, MORC2, MUC5B, NKX6-3, NOP16, PDYN, PLIN1, PNO1, RASIP1, RBMXL2, RBMXL3, RFX4, RFX6, SLC18A1, SRSF8, TCOF1, and ZNF439. One autoantigen target is KHDC3L and anti-KHDC3L autoantibodies are linked with premature ovarian failure in APS1 patients. Another autoantigen target is RFX6, and anti-RFX6 autoantibodies are linked with diarrheal-type intestinal dysfunction in APS1 patients. Another autoantigen target is PLIN1, and anti-PLIN1 autoantibodies are linked with lipodystrophy in APS1 patients. Yet another autoantigen target is ACPT, and anti-ACPT autoantibodies are linked with dental enamel hypoplasia in APS1 patients. Other new APS1 autoantibodies are shown in this disclosure to be associated with other APS1 associated conditions as shown in FIG. 5.

TABLE 1
Selected Novel APS1 Autoantigens

| | UniProt/UniProtKB | |
|---|---|---|
| Autoantigen | Database Entry No. | Sequence |
| ACPT/ACP4 | Q9BZG2 | SEQ ID NO: 29 |
| AOAH | P28039 | SEQ ID NO: 1 |
| ARFRP1 | Q13795 | SEQ ID NO: 2 |
| ARRDC3 | Q96B67 | SEQ ID NO: 3 |
| C7orf50 | Q9BRJ6 | SEQ ID NO: 4 |
| CAMK2N1 | Q7Z7J9 | SEQ ID NO: 5 |
| CDK2 | P24941 | SEQ ID NO: 6 |
| DAZ1 | Q9NQZ3 | SEQ ID NO: 7 |
| DAZ4 | Q86SG3 | SEQ ID NO: 8 |
| DEDD2 | Q8WXF8 | SEQ ID NO: 9 |
| GET4 | Q7L5D6 | SEQ ID NO: 10 |
| GIP | P09681 | SEQ ID NO: 11 |
| HAPLN1 | P10915 | SEQ ID NO: 12 |
| KHDC3L | Q587J8 | SEQ ID NO: 13 |
| MORC2 | Q9Y6X9 | SEQ ID NO: 14 |
| MUC5B | Q9HC84 | SEQ ID NO: 15 |
| NKX6-3 | A6NJ46 | SEQ ID NO: 16 |
| NOP16 | Q9Y3C1 | SEQ ID NO: 17 |
| PDYN | P01213 | SEQ ID NO: 18 |
| PNO1 | Q9NRX1 | SEQ ID NO: 19 |
| PLIN1 | O60240 | SEQ ID NO: 30 |
| RASIP1 | Q5U651 | SEQ ID NO: 20 |
| RBMXL2 | O75526 | SEQ ID NO: 21 |
| RBMXL3 | Q8N7X1 | SEQ ID NO: 22 |
| RFX4 | Q33E94 | SEQ ID NO: 23 |
| RFX6 | Q8HWS3 | SEQ ID NO: 24 |
| SLC18A1 | P54219 | SEQ ID NO: 25 |
| SRSF8 | Q9BRL6 | SEQ ID NO: 26 |
| TCOF1 | Q13428 | SEQ ID NO: 27 |
| ZNF439 | Q8NDP4 | SEQ ID NO: 28 |

This disclosure describes methods and compositions for immunohistochemically detecting the presence of the novel autoantibodies identified in this disclosure in a biological sample. In one aspect, this disclosure provides methods of detecting one or more autoantibodies in a biological sample from a subject that this is suspected of having APS1. For example, this disclosure provides methods of detecting autoantibodies in a biological sample from a subject that this is suspected of having APS1 that bind specifically to at least one of the APS1 associated autoantigens listed in Table 1. Also provided in this disclosure are methods of identifying a subject with APS1 that is at risk of developing one or more of diarrheal-type intestinal dysfunction, primary ovarian insufficiency, dental enamel hypoplasia, and/or lipodystrophy. According to some embodiments, provided are methods of identifying a subject with APS1 that is at risk of developing diarrheal-type intestinal dysfunction by detecting in a biological sample the presence of RFX6 autoantibodies. According to some embodiments, provided are methods of identifying a subject with APS1 that is at risk of developing primary ovarian insufficiency by detecting in a biological sample the presence of KHDC3L autoantibodies. According to some embodiments, provided are methods of identifying a subject with APS1 that is at risk of developing enamel hypoplasia by detecting in a biological sample the presence of ACPT autoantibodies. According to some embodiments, provided are methods of identifying a subject with APS1 that is at risk of developing lipodystrophy by detecting in a biological sample the presence of PLIN1 autoantibodies. In some instances, the subject has a loss of function mutation in the AIRE gene. This disclosure also provides methods of treating a subject that has APS1 where the subject is positive for APS1-associated autoantibodies that bind to at least one of the APS1 autoantigens listed in Table 1. In some instances, the patients are serologically positive for one or more of the autoantibodies.

APS1, also referred to as autoimmune polyendocrine syndrome type 1 or autoimmune polyendocrinopathy-candidiasis-ectodermal dystrophy (APECED), is an inherited autoimmune condition that affects many of the body's organs (OMIM #240300). Symptoms often begin in childhood or adolescence and may include mucocutaneous candidiasis, hypoparathyroidism, and Addison disease. This syndrome can cause a variety of additional signs and symptoms, such as weak teeth (enamel hypoplasia) and chronic diarrhea or constipation. Also, about 60% of the women with APS1 who are younger than 30 years of age develop primary ovarian insufficiency. Complications of APS-1 can affect the bones, joints, skin, and nails, the gonads (ovaries and testicles), the eyes, the thyroid, and several internal organs (kidneys, liver, lungs and the spleen). Anemia may also be present due to a lack of production of the red blood cells. Type 1 diabetes also occurs in some patients with this condition. Another condition that may occur in APS1 patients is lipodystrophy, which is characterized by fat loss and/or abnormal distribution of fat in the body. APS1 is progressive, with symptoms appearing at different time intervals. For example, chronic mucocutaneous candidiasis and hypoparathyroidism classically appear early in childhood, whereas adrenal insufficiency usually start in the second decade of life. Diagnosis is suspected when there are at least two of these features, particularly in young people. Treatment may include hormone-replacement, and medication for candidiasis, as well as specific treatment of any complications.

APS1 is defined by loss of function mutations in AIRE, a well-studied thymic transcriptional regulator. Inheritance is autosomal recessive. Loss-of-function mutations, also called inactivating mutations, result in the gene product having less or no function (being partially or wholly inactivated). When the allele has a complete loss of function it can be referred to as a null allele. AIRE is a transcription factor expressed in the medulla (inner part) of the thymus. The AIRE protein is part of the mechanism which eliminates self-reactive T cells that cause autoimmune disease. In the absence of AIRE, many of the tissue-restricted antigens (TSAs) fail to be expressed, resulting in aberrant T cell education to “self” and subsequent autoimmune response in the periphery. Because APS1 patients tend to develop various autoimmune manifestations over the course of many years, it is thought that each manifestation may be explained by autoimmune response to one or few initial protein targets. In principle, these target proteins would most likely (1) exhibit Aire-dependency and (2) be restricted to the single or narrow range of tissues associated with the corresponding autoimmune disease. For example, adrenal insufficiency, which results from autoimmune response to cells of the adrenal gland, is thought to occur due to targeting of adrenally-expressed cytochrome p450 family members. However, a more complete understanding of the protein target spectrum paired with clinical phenotypic associations has been lacking. This, combined with the limited applicability of murine observations to the human disease, has left the question of which clinical characteristics best associate with APS1 autoantigens a heavily debated subject.

With respect to APS1, the identification of key B cell autoantigens has occurred most commonly through candidate-based approaches and by whole-protein microarrays. For example, lung antigen BPIFB1 autoantibodies, which are commonly used to assess APS1 patients for risk of interstitial lung disease, were discovered first in Aire-deficient mice using a combination of targeted immunoblotting, tissue microscopy, and mass spectrometry (Shum et al., 2013 and 2009). Since then, a higher-throughput antibody target profiling approach utilizing a fixed protein microarray technology (ProtoArray®, ThermoFisher) has enabled detection of a wider range of proteins targeted by patient autoantibodies directly from human serum (Fishman et al., 2017; Landegren et al., 2016; Meyer et al., 2016). Despite initial success of this technology in uncovering shared antigens across cohorts of APS1 patients, it is likely that many shared antigens remain to be discovered, given that these arrays do not encompass the full coding potential of the proteome.

As described in this disclosure, the approach taken in developing the described invention merged these two approaches by (1) using serum from a cohort of well-defined patients with APS1) and high affinity autoantibodies and (2) by screening with an antigenic library that broadly covers the majority of protein coding genes. The novel APS1 autoantigen markers identified using this approach are listed in Table 1. Four of the autoantigen markers identified are RFX6, KHDC3L, ACPT, and PLIN1 which are discussed in more detail below.

The human DNA-binding protein RFX6 (RFX6) is a 928 amino acid residue protein. See UniProt Database Entry UniProt/UniProtKB Database Entry Q8HWS3. RFX6 is a transcription factor required to direct islet cell differentiation during endocrine pancreas development, including differentiation of four of the five islet cell types and for the production of insulin. Not required for pancreatic PP (polypeptide-producing) cells differentiation. Acts downstream of NEUROG3 and regulates the transcription factors involved in beta-cell maturation and function, thereby restricting the expression of the beta-cell differentiation and specification genes, and thus the beta-cell fate choice. Activates transcription by forming a heterodimer with RFX3 and binding to the X-box in the promoter of target genes. Involved in glucose-stimulated insulin secretion by promoting insulin and L-type calcium channel gene transcription. Some individual APS1 patients have been reported to feature histologic loss of intestinal enteroendocrine cells on biopsy. The association of anti-RFX6 antibodies with the diarrheal type of intestinal dysfunction discussed herein is consistent with published studies in murine models of Rfx6 (and enteroendocrine cell) ablation (Piccand et al., 2019; S. B. Smith et al., 2010). In addition, human enteroendocrine cell deficiency as well as mutations in enteroendocrine gene NEUROG3 have been linked to chronic diarrhea and malabsorption, and recently, intestinal enteroendocrine cells have been suggested to play a role in mediating intestinal immune tolerance (Ohsie et al., 2009; Sifuentes-Dominguez et al., 2019; J. Wang et al., 2006).

The human KHDC3-like protein (KHDC3L) is an 217 amino acid residue protein. See UniProt Database Entry UniProt/UniProtKB Database Entry Q587J8. Considered by a number of resources to be the ortholog of rodent Khdc3/Filia. However, sequence similarity is low and synteny is not conserved. Studies suggest that KHDC3L/ECAT1 has been lost in rodents. See Pierre, A. et al., Genomics 90(5):583-590 (2007). As described in this disclosure, an association of anti-KHDC3L antibodies with ovarian insufficiency was uncovered. Ovarian insufficiency is a disease that affects over half of all women with APS1 and manifests as abnormal menstrual cycling, reduced fertility, and early menopause. While autoreactivity to the steroidogenic granulosa cells—the cells surrounding and supporting the oocytes—has been proposed as one etiology of the clinical ovarian insufficiency, it has also been suggested that there may exist an autoimmune response to the oocyte itself (Jasti et al., 2012; Maclaren et al., 2001; Obermayer-Straub, Strassburg, & Manns, 2000; Otsuka et al., 2011; Welt, 2008). Interestingly, primary ovarian insufficiency in the absence of AIRE-deficiency is increasingly common and affects an estimated 1 in 100 women; up to half of these cases have been proposed to have autoimmune etiology (Huhtaniemi et al., 2018; Jasti et al., 2012; Nelson, 2009; Silva et al., 2014).

As described in this disclosure, the majority of patients with antibodies to KHDC3L also exhibited antibodies to NLRP5, and the majority of patients with antibodies to NLRP5 also exhibited antibodies to KHDC3L. Both of these proteins are parts of a subcortical maternal complex (SCMC) in both human and murine oocytes (Li et al., 2008; K. Zhu et al., 2015). Indeed, “multi-pronged” targeting of the same pathway has been previously implicated in APS1, where antibodies to DDC and TPH—enzymes in the melatonin synthesis pathway—have been described (Ekwall et al., 1998; Husebye et al., 1997; Kluger, Jokinen, Lintulahti, Krohn, & Ranki, 2015).

The human testicular acid phosphatse (ACPT) protein is an acid phosphatase and 426 amino acids in length. See UniProt Database Entry Q9BZG2. ACPT is expressed mainly in testis and to a lower extent in the prostate, trachea, and other tissues. As described in this disclosure, an association of anti-ACPT antibodies with dental enamel hypoplasia was uncovered. Dental enamel hypoplasia is a developmental condition characterized by undermineralization of tooth enamel, resulting in thinner and weaker enamel on adult teeth. Genetic defects in ACPT have been linked to enamel hypoplasia, presumably due to its expression and function in secretory (immature) ameloblasts where it may play a role in differentiation and mineralization of ondontblasts. The identification of antibodies to ACPT provides the first suggestion of an autoimmune etiology for some cases of dental enamel hypoplasia.

The human Perilipin-1 (PLIN1) is 522 amino acids in length and a classical lipid droplet associated protein. See UniProt Database Entry 060240. It is an abundant protein within adipocytes (fat cells) and regulates lipid homeostasis. Within adipocytes, PLIN1 is localized to specialized intracellular organelles known as lipid droplets which provide the main source of lipid storage. More specifically, PLIN1 is localized in the membrane encapsulating the lipid droplet. Here PLIN1 plays a dynamic role in regulating the total amount of lipids in lipid droplets by altering lipid droplet size or specifically recruiting enzymes that breakdown lipids. PLIN1 was discovered through candidate testing of a subset of patients with autoimmune acquired generalized lipodystrophy (AGL), providing the first link between autoimmunity and a lipodystrophy phenotype (Corvillo et al. (2018), “Autoantibodies Against Perilipin 1 as a Cause of Acquired Generalized Lipodystrophy,’ Front. Immunol. 9:2142). The association of an immune response to PLIN1 and clinical lipodystrophy is relatively new knowledge, poorly understood and non-exhaustively explored. As described in this disclosure, an association of anti-PLIN1 antibodies with APS1 associated lipodystrophy was uncovered. Lipodystrophy is a disease defined by an abnormal distribution of fat in the body. This can refer both to fat loss (lipoatrophy) and abnormal accumulation of fat tissue. A reduction in subcutaneous fat may occur in some parts of the body (partial lipodystrophy) or throughout (AGL). Akin to an excessive gain in fat, excessive fat loss can be damaging to health. The pathological loss of adipocytes leads to an imbalance of lipid metabolism, storage and signaling, which can manifest as a myriad of clinically severe outcomes, including insulin resistance from lipolysis or metabolic disorder or death. Interestingly, the Examples below show that Plin1 is expressed in the thymus in an Aire-dependent fashion, mirroring the expression pattern of other known self-antigens like Ins2 (insulin).

As described herein, an additional autoantibody-targeted enzyme ASMT, which catalyzes the final step in the melatonin synthesis pathway, was discovered. While the earlier DDC- and TPH-catalyzed steps occur primarily in the intestine, ASMT is expressed in the pineal gland, suggesting that targeting of this pathway does not occur solely due to antigen location.

As provided herein, proteins listed in Table 1 are autoantigens associated with APS1, and autoantibodies that bind specifically these proteins are valuable markers (e.g., serological markers) of a subject's immune response to the condition. Such markers are not found in healthy subjects and are accompanied by clinical symptoms and signs associated with APS1. The identity of these autoantigens permits the identification of patients that have APS1 as well as patients that are at risk of developing certain clinical manifestations, including, for example, diarrheal-type intestinal dysfunction with respect to RFX6 autoantibodies and, for example, ovarian insufficiency with respect to KHDC3L autoantibodies. Tests using individual antigenic polypeptides or fragments thereof, as well as panels of antigenic polypeptides or fragments thereof can be used to screen APS1 patients to identify those at risk of identifying conditions or sub-syndromes associated with APS1. In some cases the antigenic polypeptides is a fragment of the autoantigen that is immobilized on a solid surface or displayed on a phase display or array, to create a panel for identifying APS1 patients at risk for developing conditions associated with the disease.

### A. Methods of Detection and Compositions

In one aspect, an APS1 associated autoantigen polypeptide or fragment or variant thereof can be used in various immunological techniques to detect APS1-associated autoantibodies. Selected autoantigen polypeptides of this disclosure are listed in Table 1. The entire APS1 associated autoantigen polypeptide can used in the provided methods, a fragment of the polypeptide may be used, a variants of the polypeptide may be used, or a combination of two or more of the full length polypeptide, a fragment, or a variant thereof as described in this disclosure may be used. In some embodiments, the autoantigen polypeptide is a RFX6 polypeptide or fragments thereof. In some embodiments, the autoantigen polypeptide is a KHDC3L polypeptide. In some embodiments, the autoantigen polypeptide is an ACPT polypeptide or fragments thereof. In some embodiments, the autoantigen polypeptide is a PLIN1 polypeptide or fragments thereof. In some embodiments, one or more of the APS1 autoantigens listed in Table 1 or disclosed herein may be used in an immunoassay to detect the presence of one or more APS1-associated autoantibodies. For example, a RFX6 polypeptide or fragment or variant thereof can be used in an immunoassay to detect RFX6 autoantibodies in a biological sample. In another example, a KHDC3L polypeptide or fragment or variant thereof can be used in an immunoassay to detect KHDC3L autoantibodies in a biological sample. In another example, an ACPT polypeptide or fragment or variant thereof can be used in an immunoassay to detect ACPT autoantibodies in a biological sample. In another example, a PLIN1 polypeptide or fragment or variant thereof can be used in an immunoassay to detect PLIN1 autoantibodies in a biological sample. The autoantigen polypeptides used in an immunoassay can be in a cell lysate (such as, for example, a whole cell lysate or a cell fraction), or purified autoantigen polypeptides or fragments thereof can be used provided at least one antigenic site recognized by autoantigen-specific antibodies (such as RFX6 autoantibodies or KHDC3L autoantibodies) remains available for binding.

In one aspect, provided are methods of detecting the presence of an autoantibody in a biological sample from a subject presenting with one or more clinical symptoms associated with APS1, comprising the steps of contacting the biological sample with an autoantigen polypeptide as listed in Table 1 or fragment or variant thereof and detecting the presence of binding of the autoantigen polypeptide or fragment or variant thereof to autoantibodies that bind specifically to the autoantigen in the biological sample. In some embodiments, the autoantigen polypeptide or fragment or variant thereof is a RFX6 polypeptide or fragment or variant thereof. In some embodiments, the autoantigen polypeptide or fragment or variant thereof is a KHDC3L polypeptide or fragment or variant thereof. In some embodiments, the autoantigen polypeptide or fragment or variant thereof is an ACPT polypeptide or fragment or variant thereof. In some embodiments, the autoantigen polypeptide or fragment or variant thereof is a PLIN1 polypeptide or fragment or variant thereof.

A “biological sample,” as used herein, is generally a sample from a subject, preferably a mammalian subject. Exemplary subjects include, but are not limited to humans, non-human primates such as monkeys, dogs, cats, mice, rats, cows, horses, camels, goats, and sheep. The subject may be male or female. In some embodiments, the subject is a female human. In some embodiments, the female subject has ovaries. The subject may be a juvenile subject, such subject being a pre-pubescent subject. Alternatively, the subject may be an adult subject, including post-pubescent subjects. Non-limiting examples of biological samples include blood, serum, plasma, cerebrospinal fluid (CSF), urine. Additionally, solid tissues, for example, tissue biopsies of ovarian, intestinal, or other organs or tissues may be used.

In some embodiments, the subject has or is suspected to have APS1. In some embodiments, the subject is diagnosed with APS1. In some embodiments, the subject has been determined to have a mutation in the AIRE gene resulting in loss of function of the encoded protein. In some embodiments, the subject has one or more of nail dystrophy, hypoparathyroidism, keratoconjunctivitis, chronic mucocutaneous candidiasis, intestinal dysfunction (which can include constipation-type intestinal dysfunction or diarrheal-type intestinal dysfunction), autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis. In one example, the subject has intestinal dysfunction, particularly diarrheal-type intestinal dysfunction. The symptoms of diarrheal-type intestinal dysfunction are frequent, loose or watery stools, and a subjective sense of urgency to evacuate bowels. The symptoms of constipation-type intestinal dysfunction are infrequent bowel movements (e.g., less than 3 per week), passage of hard stools, and sometimes difficulty in passing stools. In one example, the subject has primary ovarian insufficiency (POI). Primary ovarian insufficiency (POI), also known as premature ovarian failure, refers to the loss of normal function of the ovaries in a post-pubescent female subject prior to the age of 40, which is characterized as the depletion or dysfunction of ovarian follicles with cessation of menses before age 40 years of age. Dental enamel hypoplasia refers to undermineralization of tooth enamel that results in thinner and weaker enamel on adult teeth.

Generally, the biological sample is assessed for the presence of APS1 autoantibodies by contacting it with an autoantigen polypeptide listed in Table 1 or fragment or variant thereof. For example, the biological sample can be assessed for the presence of RFX6 autoantibodies by contacting it with a RFX6 polypeptide or fragment or variant thereof. In another example, the biological sample can be assessed for the presence of KHDC3L autoantibodies by contacting it with a KHDC3L polypeptide or fragment or variant thereof. In another example, the biological sample can be assessed for the presence of ACPT autoantibodies by contacting it with an ACPT polypeptide or fragment or variant thereof. In another example, the biological sample can be assessed for the presence of PLIN1 autoantibodies by contacting it with a PLIN1 polypeptide or fragment or variant thereof. In some embodiments, an APS1 associated autoantibody that binds specifically to an autoantigen polypeptide listed in Table 1 is present in a solid tissue such as a tissue section. For example, a tissue sample comprising an RFX6 autoantibody, a KHDC3L autoantibody, an ACPT antibody, and/or a PLIN1 antibody may be used, which may be in the form of a tissue section fixed on a carrier, for example a glass slide for microscopic analysis. For example, the solid tissue can be ovarian tissue. In another example, the solid tissue can be intestinal tissue. In some embodiments, the autoantigen polypeptide or fragment thereof is present in a sample from a mammal. For example, mouse tissue is routinely used in immunohistochemistry but tissue from other rodents (e.g., rats) or other mammals (e.g., rabbits, non-human primates, or humans) also can be used in the present methods. Tissue sections used in immunohistochemistry are well known in the art and are commercially available from a number of companies (e.g., Asterand, Inc. (Detroit, Mich.); Euroimmun (Morris Plains, N.J.); and Imgenex (San Diego, Calif.)).

In other embodiments, a liquid sample from a subject that comprises an APS1-specific autoantibody that binds specifically to an autoantigen polypeptide listed in Table 1 may be used to practice the methods. Exemplary liquid samples include cell lysate, blood, serum, cerebrospinal fluid (CSF), and urine. A step of contacting a liquid sample comprising APS1-specific autoantibodies with an autoantigen polypeptide listed in Table 1 or fragment or variant thereof may be carried out by incubating an immobilized form of said polypeptide in the presence of the liquid sample under conditions that are compatible with the formation of a complex comprising said polypeptides and said autoantibodies. Optionally, the liquid sample, then at least partially depleted of autoantibodies may subsequently be removed to facilitate detection of a complex between the autoantibodies and the autoantigen polypeptide or fragment thereof. Optionally, one or more washing steps may be contemplated. In some embodiments, the liquid sample comprises RFX6 autoantibodies and is contacted with a RFX6 polypeptide or fragment thereof. In some embodiments, the liquid sample comprises KHDC3L autoantibodies and is contacted with a KHDC3L polypeptide or fragment thereof. In some embodiments, the liquid sample comprises ACPT autoantibodies and is contacted with an ACPT polypeptide or fragment thereof. In some embodiments, the liquid sample comprises PLIN1 autoantibodies and is contacted with a PLIN1 polypeptide or fragment thereof.

In some embodiments, the autoantigen polypeptide listed in Table 1 or fragment thereof is an isolated, purified polypeptide or fragment thereof as discussed below. For example, the autoantigen polypeptide or fragment thereof may be an isolated, purified RFX6 polypeptide or fragment thereof. In another example, the autoantigen polypeptide or fragment thereof may be an isolated, purified KHDC3L polypeptide or fragment thereof. In another example, the autoantigen polypeptide or fragment thereof may be an isolated, purified ACPT polypeptide or fragment thereof. In another example, the autoantigen polypeptide or fragment thereof may be an isolated, purified PLIN1 polypeptide or fragment thereof. In some embodiments, the autoantigen polypeptide or fragment thereof is in a phage display or eukaryotic cell display library. In some embodiments, the autoantigen polypeptide or fragments thereof is heterologously-expressed on the surface of a cell.

In some embodiments, the biological sample is contacted with an autoantigen polypeptide listed in Table 1 or fragment thereof and a secondary antibody. As is well known in the art, the secondary antibody is an antibody raised against the IgG of the animal species in which the primary antibody originated. Secondary antibodies bind to the primary antibody to assist in detection, sorting and purification of target antigens to which a specific primary antibody is first bound. The secondary antibody must have specificity both for the antibody species as well as the isotype of the primary antibody being used. If an APS1-specific autoantibody is present in the biological sample, under appropriate conditions, a complex is formed between the autoantigen polypeptide listed in Table 1 or fragment thereof, the autoantibody in the biological sample, and the secondary antibody. For example, if the autoantigen polypeptide is a RFX6 polypeptide or a fragment thereof, and RFX6 autoantibodies are present in the biological sample, a complex would be formed between the RFX6 polypeptide or a fragment thereof, the RFX6 autoantibodies, and the secondary antibody. In another example, if the autoantigen polypeptide is a KHDC3L polypeptide or a fragment thereof, and KHDC3L autoantibodies are present in the biological sample, a complex would be formed between the KHDC3L polypeptide or a fragment thereof, the KHDC3L autoantibodies, and the secondary antibody. In another example, if the autoantigen polypeptide is an ACPT polypeptide or a fragment thereof, and ACPT autoantibodies are present in the biological sample, a complex would be formed between the ACPT polypeptide or a fragment thereof, the ACPT autoantibodies, and the secondary antibody. In another example, if the autoantigen polypeptide is a PLIN1 polypeptide or a fragment thereof, and PLIN1 autoantibodies are present in the biological sample, a complex would be formed between the PLIN1 polypeptide or a fragment thereof, the PLIN1 autoantibodies, and the secondary antibody.

A complex comprising the APS1-specific autoantibodies and the APS1 autoantigen polypeptides listed in Table 1 or fragments thereof may be detected using a variety of methods known to the person skilled in the art, for example immunofluorescence microscopy or spectroscopy, luminescence, NMR spectroscopy, immunodiffusion, radioactivity, chemical crosslinking, surface plasmon resonance, native gel electrophoresis, or enzymatic activity. Depending on the nature of the sample, either or both immunoassays and immunocytochemical staining techniques may be used. Enzyme-linked immunosorbent assays (ELISA), Western blot, and radioimmunoassays are methods used in the art, and can be used as described herein to detect the presence of APS1-specific autoantibodies in a biological sample. While some of these methods allow for the direct detection of the complex, in some embodiments, the second antibody is labeled such that the complex may be detected specifically owing to intrinsic properties of the label such as, for example, fluorescence, radioactivity, enzymatic activity, visibility in NMR, or MRI spectra or the like. In some embodiments, the detection method may include any of Western blot, dot blot, protein microarray, ELISA, line blot radioimmune assay, immunoprecipitation, indirect immunofluorescence microscopy, radioimmunoassay, radioimmunodiffusion, ouchterlony immunodiffusion, rocket immunoelectrophoresis, immunohistostaining, complement fixation assay, FACS, and protein chip, but is not limited thereto. Methods and compositions are described herein that can be used for detecting, by immunohistochemistry, the presence of APS1-specific autoantibodies in a biological sample. Immunohistochemical methods are well known in the art, and non-limiting exemplary methods are described in U.S. Pat. Nos. 5,073,504; 5,225,325; and 6,855,552. See also Dabbs, Diagnostic Immunohistochemistry, 2^{nd} Ed., 2006, Churchill Livingstone; and Chu & Weiss, Modern Immunohistochemistry, 2009, Cambridge University Press. It would be understood by those skilled in the art that immunohistochemistry routinely includes steps that are not necessarily discussed herein in detail such as washing the tissue samples to remove unbound secondary antibodies and the parallel staining experiments with proper controls. Exemplary detection methods are described in the Examples of this disclosure, including radiolabeled ligand binding assays, immunofluorescence, and cell-based expression assays. While particular protocols are described below, variations of these assays are routine and known in the art.

In some instances, the secondary antibody is conjugated to a detectable label. Detectable labels are well known in the art and include, without limitation, a fluorescent label, an enzymatic label, a radioactive label, a luminescent label, or an affinity tag such as biotin or streptavidin. Exemplary fluorescent dyes include water-soluble rhodamine dyes, fluoresceins, 2′,7′-dichlorofluoresceins, fluorescein isothiocyanate (FITC), DyLight™ 488, phycoerythrin (PE), propidium iodide (PI), PerCP, PE-Alexa Fluor® 700, Cy5, allophycocyanin, Cy7, benzoxanthene dyes, and energy transfer dyes, as disclosed in the following references: Handbook of Molecular Probes and Research Reagents, 8th ed. (2002), Molecular Probes, Eugene, Oreg.; U.S. Pat. Nos. 6,191,278, 6,372,907, 6,096,723, 5,945,526, 4,997,928, and 4,318,846; and Lee et al., 1997, Nucleic Acids Research 25:2816-2822. Exemplary enzymatic labels include but are not limited to alkaline phosphatase (AP) and horseradish peroxidase (HP)). Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Suitable radioactive labels include, for example, ³²P, ³³P, ¹⁴C, ¹²⁵I, ¹³¹I, ³⁵S, and ³H. In some instances, the detectable label can be a heterologous polypeptide such as an antigenic tag such as, for example, FLAG, polyhistidine, hemagglutinin (HA), glutathione-S-transferase (GST), or maltose-binding protein (MBP)) for use in purifying the APS1 autoantigen polypeptides or fragments or variants thereof. In some instances, the detectable label can be a heterologous polypeptide that is useful as diagnostic or detectable marker such as, for example, luciferase, a fluorescent protein (such as a green fluorescent protein (GFP)), or chloramphenicol acetyl transferase (CAT). Another labeling technique which may result in greater sensitivity is the coupling of the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

In some embodiments, the method comprises contacting an autoantigen polypeptide listed in Table 1 or fragment or variant thereof with a biological sample from a subject and a secondary antibody having a suitable label thereon under conditions in which a complex is formed between the autoantigen polypeptide or fragment or variant thereof, a corresponding APS1-specific autoantibody in the biological sample that binds specifically to the autoantigen polypeptide or fragment of variant thereof, if present, and the secondary antibody; and detecting the complex formed, if formed, by detecting the label of the secondary antibody, wherein the presence of the secondary antibody is indicative of the presence of a APS1-specific autoantibody in the biological sample that binds specifically to the autoantigen polypeptide or fragment or variant thereof, and wherein the absence of the secondary antibody is indicative of the absence of a APS1-specific autoantibody in the biological sample that binds specifically to the autoantigen polypeptide or fragment or variant thereof. In some instances, the secondary antibody is detectably-labeled. Immobilization of an autoantigen polypeptide listed in Table 1 or fragment or variant thereof on a solid carrier can facilitate the method of APS1-specific autoantibody detection as discussed below. In some instances, the method comprises contacting an autoantigen polypeptide listed in Table 1 or fragment or variant thereof having a suitable label thereon with a biological sample from a subject, and immunoprecipitating any complex formed between the autoantigen polypeptide or fragment or variant thereof and a corresponding autoantibody in the biological sample, and monitoring for said label on any of said complexes, wherein the presence of said label is indicative of the presence of a autoantibody in the biological sample that binds specifically to said autoantigen polypeptide and the absence of said label is indicative of the absence of a autoantibody in the biological sample that binds specifically to said autoantigen polypeptide. In some instances, the method comprises a combination of immunoprecipitation and Western blot analysis to detect the presence of a APS1-specific autoantibody in a biological sample from a subject. For example, the method may comprise contacting an APS1 autoantigen polypeptide listed in Table 1 or fragment or variant thereof with a biological sample from a subject under conditions in which a complex is formed between the autoantigen polypeptide or fragment or variant thereof and a corresponding APS1-specific autoantibody in the biological sample, if present; immunoprecipitating any complex formed between the autoantigen polypeptide or antigenic fragment or variant thereof and a corresponding APS1-specific autoantibody in the biological sample to produce an immunoprecipitate comprising any such complex formed; separating components of the immunoprecipitate from each other (e.g., by electrophoresis), said components comprising the autoantigen polypeptide or fragment or variant thereof and a corresponding APS1-specific autoantibody in the biological sample, if present; and contacting the components of the immunoprecipitate with a secondary antibody having a suitable label thereon that specifically binds to a constant region of the APS1-specific autoantibody, if present; and detecting the complex formed, if formed, by detecting the label of the secondary antibody, wherein the presence of the secondary antibody is indicative of the presence of an APS1-specific autoantibody in the biological sample, and wherein the absence of the secondary antibody is indicative of the absence of an APS1-specific autoantibody in the biological sample. Exemplary labels include any of the detectable labels described in this disclosure including, for example, fluorescent dyes and radioactive labels.

In some embodiments, a APS1 autoantigen polypeptide listed in Table 1 or fragment or variant thereof is heterologously-expressed on the surface of a cell. For example, a vector comprising the coding sequence of the autoantigen polypeptide or fragment or variant thereof operably linked to a promoter can be introduced into a cell. The vector may comprise elements that cause the autoantigen polypeptide or fragment or variant thereof to be expressed on the surface of the cell. For example, autoantigen polypeptides, fragments, and variants may be expressed as fusion proteins with a membrane protein on the surface of the cell. In some instances, the cell is a bacteria cell or a eukaryotic cell. For example, the eukaryotic cell may be a yeast cell or a mammalian cell such as a human cell. Methods of transfection and transduction of cells to introduce recombinant nucleic acids are well known in the art.

In some embodiments, the APS1 autoantigen polypeptide listed in Table 1 or fragment or variant thereof is an isolated polypeptide or fragment or variant thereof. Protein expression and purification methods are well known in the art. In some embodiments, the isolated and purified autoantigen polypeptide is a RFX6 polypeptide having the amino acid residue sequence represented by UniProt/UniProtKB Database Entry No. Q8HWS3 (SEQ ID NO:24). In some embodiments, the isolated autoantigen polypeptide is a KHDC3L polypeptide having the sequence represented by UniProt/UniProtKB Database Entry No. Q587J8 (SEQ ID NO:13). In some embodiments, the isolated autoantigen polypeptide is an ACPT polypeptide having the sequence represented by UniProt/UniProtKB Database Entry No. Q9BZG2 (SEQ ID NO:29). In some embodiments, the isolated autoantigen polypeptide is a PLIN1 polypeptide having the sequence represented by UniProt/UniProtKB Database Entry No. 060240 (SEQ ID NO:30). These and all sequence accession numbers referred to throughout this application, refers to the sequence deposited in the UniProtKB and NCBI databases, more specifically the versions publicly available on Dec. 19, 2019. However, the teachings of the present disclosure may not only be carried out using polypeptides having the exact amino acid sequences referred to in this application explicitly, for example by name, sequence or accession number, or implicitly, but also using fragments or variants of such polypeptides. Thus, modified versions of the APS1 autoantigen polypeptide listed in Table 1 and fragments and variants thereof are also contemplated, such as those in which one or more amino acid residues are substituted or modified (such as with glutaraldehyde).

An “isolated” or “purified” polypeptide, or portion thereof, is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the APS1 autoantigen polypeptide or antigenic portion thereof is recombinantly produced, optimally culture medium represents less than about 30%, 20%, 10%, 5%, or 1% (by dry weight) of chemical precursors or non-protein-of-interest chemicals.

The term “fragment”, with regard to the autoantigen polypeptides listed in Table 1 as described in this disclosure, refers to an amino acid residue sequence of a portion of the full length protein, encompassing, for example, an amino acid residue sequence that is truncated at one or both termini by one or more amino acids. Within the context of this disclosure, a fragment of an autoantigen polypeptide listed in Table 1 retains its antigenicity such that it is specifically bound under appropriate conditions by an APS1 autoantibody that would specifically bind to the corresponding full length autoantigen polypeptide under appropriate conditions. An antigenic portion of the autoantigen proteins listed in Table 1 can be a polypeptide that is, for example, 10, 25, 50, 100, 150, 200, 250 or more amino acid residues in length of the full length protein. For example, with respect to RFX6 protein fragments and KHDC3L protein fragments, the fragments may be 10, 25, 50, 100, 150, 200, 250 or more amino acid residues in length of the full length proteins as set forth in SEQ ID NOs: 24 or 13, respectively. In another example, with respect to the ACPT protein fragments, the fragments may be 10, 25, 50, 100, 150, 200, 250 or more amino acid residues in length of the full length proteins as set forth in SEQ ID NOs: 29. In another example, with respect to the PLIN1 protein fragments, the fragments may be 10, 25, 50, 100, 150, 200, 250 or more amino acid residues in length of the full length proteins as set forth in SEQ ID NOs: 30. In some embodiments, an autoantigen polypeptide sequence may comprise one or more internal deletions of one or more amino acid residues. The residual length of the fragment equals or exceeds the length of one or more continuous or conformational epitopes, e.g., 6, 7, 8, 9, 10, 11, 12, 13, 14, 15, 16, 17, 18, 19, 20, 21, 22, 23, 24, 25, 26, 27, 28, 29, 30, 21, 32, 33, 34, 35, 36, 37, 38, 39, 40, 41, 42, 43, 44, 45, 46, 47, 48, 49, 50, 55, 60, 65, 70, 75, 80, 85, 90, 95, 100 or more amino acid residues. In some embodiments, a fragment comprises at least 6 contiguous amino acid residues of at least one of SEQ ID NOs: 1-30. In some embodiments, a fragment comprises at least 8 contiguous amino acids of at least one of SEQ ID NOs: 1-30. In some embodiments, a fragment comprises at least 12 contiguous amino acids of at least one of SEQ ID NOs: 1-28. In some embodiments, a fragment comprises 8-12 contiguous amino acids of at least one of SEQ ID NOs: 1-30. In some embodiments, a fragment comprises 30-60 contiguous amino acids of at least one of SEQ ID NOs: 1-30. In some embodiments, a fragment comprises 47 contiguous amino acids of at least one of SEQ ID NOs: 1-30, such as, for example, one or more of the sequences set forth in SEQ ID NO:13, SEQ ID NO:24, SEQ ID NO:29, and/or SEQ ID NO:30. In some instances, a plurality of fragments is provided, each fragment comprising 49 contiguous amino acids of any one of SEQ ID NOs: 13, 24, 29, and/or 30. In some instances, a plurality of fragments is provided, each fragment comprising 48 contiguous amino acids of any one of SEQ ID NOs: 13, 24, 29, and/or 30 as identified in Table 2. In some instances, a plurality of fragments is provided, each fragment comprising 48 contiguous amino acids of any one of SEQ ID NOs: 13, 24, 29, and/or 30 as identified in Table 6. For example, a first fragment may comprise amino acids 1-49 of SEQ ID NO:13, a second fragment comprising amino acids 25-73, a third fragment comprising amino acids 49-97 of SEQ ID NO:13, and so on with each additional fragment having a first amino acid residue 22 amino acids downstream in the amino acid sequence of SEQ ID NO:13 relative to the first amino acid residue of the prior fragment. In another example, a first fragment may comprise amino acids 1-49 of SEQ ID NO:24, a second fragment comprising amino acids 25-73, a third fragment comprising amino acids 49-97 of SEQ ID NO:24, and so on with each additional fragment having a first amino acid residue 22 amino acids downstream in the amino acid sequence of SEQ ID NO:24 relative to the first amino acid residue of the prior fragment. In another example, a first fragment may comprise amino acids 1-49 of SEQ ID NO:29, a second fragment comprising amino acids 25-73, a third fragment comprising amino acids 49-97 of SEQ ID NO:29, and so on with each additional fragment having a first amino acid residue 22 amino acids downstream in the amino acid sequence of SEQ ID NO:29 relative to the first amino acid residue of the prior fragment. In another example, a first fragment may comprise amino acids 1-49 of SEQ ID NO:30, a second fragment comprising amino acids 25-73, a third fragment comprising amino acids 49-97 of SEQ ID NO:30, and so on with each additional fragment having a first amino acid residue 22 amino acids downstream in the amino acid sequence of SEQ ID NO:30 relative to the first amino acid residue of the prior fragment. Antigenic RFX6 polypeptide fragments include those comprising the sequence of any of SEQ ID NOs: 31-37 or more than one thereof. Antigenic KHDC3L polypeptide fragments include those comprising the sequence of any of SEQ ID NOs: 38-40 or more than one thereof. Antigenic ACPT polypeptide fragments include those comprising the sequence of SEQ ID NO: 41. Antigenic PLIN1 polypeptide fragments include those comprising the sequence of any of SEQ ID NOs: 42-46 or more than one thereof. The person of skill in the art is familiar with guidelines used to design peptides having sufficient immunogenicity such as, for example, those described in Jackson, D. C., et al., Vaccine 18(3-4): 355-361 (1999) and Black, M., et al., Expert Rev. Vaccines, 9(2): 157-173 (2010). Briefly, it is desirable that the peptide meets as many as possible of the following requirements: (a) it has a high degree of hydrophilicity, (b) it comprises one or more residues selected from the group comprising aspartate, proline, tyrosine, and phenylalanine, (c) is has, for higher specificity, no or little homology with other known peptides or polypeptides, (d) it is sufficiently soluble, and (e) it comprises no glycosylation or phosphorylation sites unless required for specific reasons. Alternatively, bioinformatics approaches may be followed such as, for example, those described by Moreau, V., et al., BMC Bioinformatics 2008, 9:71 (2008). Such biologically active portions can be prepared by recombinant techniques and evaluated for pesticidal activity.

TABLE 2
Antigenic polypeptide fragments for RFX6,
KHDC3L, and ACPT.

| | | SEQ ID |
|---|---|---|
| Protein | Amino acid sequence | NO |
| RFX6 | PLPSSQPGGLGPALHQFPAGNTDNMPLTGQMEL | 31 |
| | SQIAGHLMTPPISPA | |
| RFX6 | MVNQHVSVISSIRSLPPYSDIHDPLNILDDSGR | 32 |
| | KQTSSFYTDTSSPVA | |
| RFX6 | MPLTGQMELSQIAGHLMTPPISPAMASRGSVIN | 33 |
| | QGPMAGRPPSVGPVL | |
| RFX6 | NRNKGSMVSSDAVKNESHVETTYLPLPSSQPGG | 34 |
| | LGPALHQFPAGNTDN | |
| RFX6 | AGSPYNSRPPSSYGPSLQAQDSHNMQFLNTGSF | 35 |
| | NFLSNTGAASCQGAT | |
| RFX6 | SAPSHCSTYPEPIYPTLPQANHDFYSTSSNYQT | 36 |
| | VFRAQPHSTSGLYPH | |
| RFX6 | TKAADQYLSQKKTITQIVKDKKKQTQLTLQWLE | 37 |
| | ENYIVCEGVCLPRCI | |
| KHDC3L | GSPVEVQEAGTQQSLQAANKSGTQRSPEAASKA | 38 |
| | VTQRFREDARDPVTR | |
| KHDC3L | TQRSVEVREAGTQRSVEVQEVGTQGSPVEVQEA | 39 |
| | GTQQSLQAANKSGTQ | |
| KHDC3L | ALAQDVATQKAETQRSSIEVREAGTQRSVEVRE | 40 |
| | AGTQRSVEVQEVGTQ | |
| ACPT | TGLSLVGEPLRRAWKVLDTLMCQQAHGLPLPAW | 41 |
| | ASPDVLRTLAQISAL | |

The term “variant” with respect to the autoantigen polypeptides or fragments thereof refers to a polypeptide comprising an amino acid residue sequence that is at least 70, 75, 80, 85, 90, 92, 94, 95, 96, 97, 98 or 99% identical to the normal sequence of the autoantigen proteins listed in Table 1 or fragments thereof. Within the context of this disclosure, a variant of an autoantigenic polypeptide listed in Table 1 retains its antigenicity such that it is specifically bound under appropriate conditions by an APS1 autoantibody that would specifically bind to the corresponding full length autoantigen polypeptide under appropriate conditions. In some instances, variants are modified at amino acid residues other than those essential for the ability of the polypeptide to be specifically bound by an antibody that binds specifically to the full length normal polypeptide sequence. In some instances, one or more such essential amino acids may optionally be replaced in a conservative manner or additional amino acids may be inserted such that the antigenicity of the variant polypeptide is preserved. The state of the art comprises various methods that may be used to align two given nucleic acid or amino acid sequences and to calculate the degree of identity, see for example Arthur Lesk (2008), Introduction to bioinformatics, Oxford University Press, 2008, 3rd edition. In a preferred embodiment, the ClustalW software (Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007). Clustal W and Clustal X version 2.0. Bioinformatics, 23, 2947-2948) is used.

Such variants of autoantigen polypeptides and fragments thereof of this disclosure may be prepared, for example, by introducing deletions, insertions or substitutions in nucleic acid sequences encoding them, or by chemical synthesis or modification. Moreover, variants of autoantigen polypeptides and fragments thereof may also be generated by fusion with other known polypeptides or variants thereof and encompass active portions or domains, preferably having a sequence identity of at least 70, 75, 80, 85, 90, 92, 94, 95, 96, 97, 98 or 99% when aligned with the active portion of the reference sequence, wherein the term “active portion”, as used herein, refers to an amino acid sequence, which is less than the full length amino acid sequence or, in the case of a nucleic acid sequence, codes for less than the full length amino acid sequence, respectively, but retains at least some of the biological activity. For example, an active portion an antigenic polypeptide retains the ability to bind to an antibody or autoantibody and, preferably, when administered to mammals, causes an immune response to occur.

The APS1 autoantigen polypeptides listed in Table 1 and fragments and variants thereof may be provided in any form and at any degree of purification, from tissues or cells comprising said polypeptides in an endogenous form, such as cells overexpressing the polypeptide and crude or enriched lysates of such cells, to purified and/or isolated polypeptides that are essentially pure. In embodiments, the autoantigen polypeptides and fragments and variants thereof have a native configuration, wherein the term “native configuration”, as used herein, refers to a folded polypeptide, such as a folded polypeptide purified from tissues or cells, such as mammalian cells or tissues or from non-recombinant tissues or cells. In another embodiment, the one or more autoantigen polypeptides and fragments and variants thereof are recombinant protpolypeptides eins, wherein the term “recombinant”, as used herein, refers to a polypeptide produced using genetic engineering approaches at any stage of the production process, for example by fusing a nucleic acid encoding the polypeptide to a strong promoter for overexpression in cells or tissues or by engineering the sequence of the polypeptide itself. Such techniques are well known in the art.

In some instances, an autoantigen polypeptide as listed in Table 1 or fragments or variants thereof can be denatured such as by heating, freezing or ultraviolet ray, or chemical treatments such as a surfactant or a denaturant. For example, such a denatured form may be prepared by treating them with sodium dodecyl sulfate (SDS) or dithiothreitol (DTT). Autoantigen polypeptides or antigenic fragments or variants thereof that are included in a kit or a panel as described herein can be provided within a cell, in a solution in which they are soluble, or the autoantigen polypeptides or fragments or variants thereof can be provided in a lyophilized form.

In some embodiments, a autoantigen polypeptide as listed in Table 1 or fragments or variants thereof can be immobilized on a solid carrier insoluble in an aqueous solution, such as via a covalent bond, electrostatic interactions, encapsulation or entrapment, for example by denaturing a globular polypeptide in a gel, or via hydrophobic interactions such as via one or more covalent bonds. Various suitable carriers, for example paper, metal, silicon or glass surfaces, microfluidic channels, membranes, beads such as magnetic beads, column chromatography media, biochips, polyacrylamide gels and the like have been described in the literature, for example in Kim, D., Herr, A. E. (2013), Protein immobilization techniques for microfluidic assays, Biomicrofluidics 7(4), 041501. This way, the immobilized molecule, together with the insoluble carrier, may be separated from an aqueous solution in a straightforward manner, for example by filtration, centrifugation or decanting. An immobilized molecule may be immobilized in a reversible or irreversible manner. For example, the immobilization is reversible if the molecule interacts with the carrier via ionic interactions that can be masked by addition of a high concentration of salt or if the molecule is bound via a cleavable covalent bond such as a disulfide bridge which may be cleaved by addition of thiol-containing reagents. By contrast, the immobilization is irreversible if the molecule is tethered to the carrier via a covalent bond that cannot be cleaved in aqueous solution, for example a bond formed by reaction of an epoxide group and an amine group as frequently used to couple lysine side chains to affinity columns. The protein may be indirectly immobilized, for example by immobilizing an antibody or other entity having affinity to the molecule, followed by formation of a complex to the effect that the molecule-antibody complex is immobilized. Various ways to immobilize molecules are described in the literature such as, for example, in Kim and Herr (2013). In addition, various reagents and kits for immobilization reactions are commercially available such as, for example, from Pierce Biotechnology.

In some embodiments, an autoantigen polypeptide as listed in Table 1 or fragment thereof is present in a tissue section, and the method comprises contacting the tissue section with a biological sample and a detectably-labeled secondary antibody under conditions in which a complex is formed between APS1 autoantigen polypeptides in the tissue section, a corresponding autoantibody in the biological sample, if present, and the detectably-labeled secondary antibody; and (b) identifying a pattern of complex formation in the tissue sample by detecting the detectably-labeled secondary antibody, wherein the presence of a pattern of complex formation is indicative of the presence of APS1-specific autoantibodies in the biological sample, and wherein the absence of a pattern of complex formation is indicative of the absence of APS1-specific autoantibodies in the biological sample.

In instances where an APS1 autoantigen polypeptide as listed in Table 1 or fragment or variant thereof is in a phage display or eukaryotic cell display library, the presence of an APS1-specific autoantibody in a biological sample from a subject is assessed by contacting the biological sample with a phage display or eukaryotic cell display library. An appropriate display library includes a plurality of eukaryotic cells or phage that express a plurality of peptides including fragments of the an autoantigen polypeptide as listed in Table 1 or variants thereof (see discussion in Section B below regarding fragments and variants) on the surface of the eukaryotic cells or phage. For example, autoantigen fragments and/or variants may be expressed as fusion proteins with a membrane protein on the surface of the eukaryotic cells or phage. Each cell or phage in the library expresses a different peptide. In some instances, the eukaryotic cell may be a yeast cell or a mammalian cell such as a human cell. The biological sample can be assayed to detect whether there is specific protein-protein interaction with any of the peptides expressed on the surface of the eukaryotic cells or phage. Methods of detecting protein-protein interactions using phage display are well-known in the art. For example, the putative autoantibody may be bound to a solid support and the phage library applied thereto. After washing the solid support, any phage that remain bound to the solid support generally expresses an APS1 autoantigen fragment or variant. The phage DNA is isolated (after bacterial amplification) and sequenced to identify the sequence of the peptide expressed by the phage. Such peptides may then be further assessed individually for specific binding to the putative utoantibody such as, for example, by immunoprecipitation, Western blot, or other immunoassay. In some instances, where the display library comprises eukaryotic cells, specific protein-protein interaction with any of the peptides may be assessed by flow cytometry. In some instances, the eukaryotic cells of the display library may be yeast cells. In some instances, the eukaryotic cells of the binding pool may be mammalian cells such as human cells. The peptides expressed on the cells of the display library may be fluorescently labeled (see discussion above regarding detectably-labeled secondary antibodies for exemplary fluorescent labels). The biological sample and the display library may be combined, and FACS analysis performed to identify cells that express peptides that are bound specifically to an APS1-specific autoantibody. In some instances, the identified cells may then be expanded in vitro, and the DNA or the RNA analyzed, such as by next generation sequencing. In some instances, single cell PCR may be performed followed by RNA and/or DNA sequence analysis. Other exemplary methods for assessing protein-protein interactions between a biological sample that contains an APS1- specific autoantibody and a display library include those described in Jardine, J., et al., 2013, Science 340(6133):711-716 and McGuire, A. T., et al., 2014, J. of Virology 88(5):2645-2657, both of which are incorporated by reference in their entireties herein. In one embodiment, the phage display system may be used such as that described in Example 1 and WO 2020/190700, which is incorporated by reference herein in its entirety for all purposes.

In some instances, more than one of the detection methods described above may be used in a complementary manner for more reliable results. In some embodiments, other immunoassays can be performed either in alternative to or before and/or after the immunohistochemistry methods. For example, a Western blot may be performed using, for example, a panel of known antigens associated with autoantibodies, the panel including one or more of the APS1 autoantigen polypeptides listed in Table 1 as provided in this disclosure, and/or antigenic fragments or variants thereof, the results of which may warrant further evaluation using, for example, the immunohistochemistry methods described herein. In another example, an immunohistochemistry method as described herein may be performed, followed by a Western blot in order to, for example, further confirm the specific antigens, including the APS1 autoantigen polypeptides provided in this disclosure, recognized by the APS1-specific autoantibodies in the biological sample. In another example, a phage or eukaryotic cell display library that includes a plurality of eukaryotic cells or phage that express a plurality of peptides including fragments and/or variants of one or more APS1 autoantigen polypeptides listed in Table 1 on the surface of the eukaryotic cells or phage may be used to assess for the presence of APS1-specific autoantibodies in the biological sample from the subject, and then followed by a radioligand binding assay method as described herein for confirmation. In another example, the biological sample may be assessed by a radioligand binding assay method first, with confirmation by assessing the sample using a phage or eukaryotic cell display library.

Any data demonstrating the presence or absence of an APS1-specific autoantibody through binding to the an APS1 autoantigen polypeptide listed in Table 1 or fragment or variant thereof may be correlated with reference data. For example, detection of an APS1-specific autoantibody in a biological sample indicates that the subject who provided the sample has APS1. In another example, detection of an APS1-specific autoantibody in a biological sample indicates that the subject who provided the sample is at risk of developing an APS1 associated condition such as, for example, diarrheal-type intestinal dysfunction or primary ovarian insufficiency. In some embodiments, a subject who is determined to have an APS1 specific autoantibody may be at risk of developing dental enamel hypoplasia. In some embodiments, a subject who is determined to have an APS1 specific autoantibody may be at risk of developing lipodystrophy. In some embodiments, a subject who is determined to have an APS1 specific autoantibody may be at risk of developing an APS1 associated condition identified in FIG. 5, which identifies which APS1 autoantigens are associated with various APS1 associated conditions. If the subject has been previously diagnosed, the amount of APS1-specific autoantibodies detected at the time of prior diagnosis and in the present time may be correlated to assess the progression of the disease and/or the success of a treatment. For example, if the amount of APS1-specific autoantibodies is found to increase, it may indicate that the disease is progressing and/or that the treatment attempted is unsuccessful.

Kits and devices useful for performing the methods of Section A are described below in Section B.

### B. Kits and Devices

In another aspect, provided in this disclosure are kits and panels containing one or more APS1 autoantigen polypeptides as listed in Table 1 or antigenic fragments or variants thereof, as provided herein, to which APS1-specific autoantibodies as described herein can specifically bind. The kits and panels are designed such that they include one or more polypeptides that are antigenic so as to be specifically bound by APS1-specific autoantibodies present in biological samples from subjects. In some instances, the kits include a panel as provided herein, such as a diagnostic panel. In some embodiments, the kits and panels include at least one polypeptide listed in Table 1 or one or more antigenic fragments or variants thereof. In some embodiments, the kits and panels include an RFX6 polypeptide or one or more antigenic fragments or variants thereof. In some embodiments, the kits and panels include a KHDC3L polypeptide or one or more antigenic fragments or variants thereof. In some embodiments, the kits and panels include an ACPT polypeptide or one or more antigenic fragments or variants thereof. In some embodiments, the kits and panels include a PLIN1 polypeptide or one or more antigenic fragments or variants thereof.

In certain embodiments, a kit as described herein includes one or more solubilizing agents for increasing the solubility of a polypeptide such as, for example, a buffer solution. The kit may further include reagents provide a detectable signal when used in conjunction with the APS1 autoantigen polypeptides or fragments or variants thereof and a biological sample. In some embodiments, the kit includes a detectably-labeled secondary antibody that is able to bind to an APS1-specific autoantibody specifically binding to an APS1 autoantigen polypeptide or fragment or variant thereof. Reagents for the detection of the secondary antibody label can also be included in the kit. The secondary antibody is detected by a method that depends on a labeling group used. Exemplary labels for secondary antibodies are described above in this disclosure.

In addition, a kit can include directions for using the APS1 autoantigen polypeptide listed in Table 1 or fragments or variants thereof and/or directions for practicing a method described herein to detect APS1-specific autoantibodies in a biological sample. The concentration or amount of APS1-specific autoantibodies contained in the biological sample is indirectly measured by measuring the amount of the detectable label. The obtained measurement value may be converted to a relative or absolute concentration, amount, activity, etc. using a calibration curve or the like.

In some embodiments, a kit or a panel as provided herein include a reference sample, such as a normal control sample. In some embodiments, a kit or a panel as provided herein include a positive control sample from a subject known to have APS1 and having one or more particular APS1 associated conditions, such as a normal control sample. For example, the positive control sample may be from a subject having APS1 that has positive ovarian insufficiency. In another example, the positive control sample may be from a subject having APS1 that has intestinal dysfunction, particularly diarrheal-type intestinal dysfunction. In some embodiments, a kit or a panel as provided herein includes one or more control antibody that detects an antigen that is expected to be present in a biological sample in a biological sample from a healthy subject and/or a subject having APS1. If one or more reference samples is included, the obtained measurement values for such samples can be compared with the results of the test sample, so that the determination of the subject's condition can be more objectively determined.

In addition to one or more APS1 autoantigen polypeptides listed in Table 1 or fragments and/or variants the panel can include additional polypeptides such as, for example, positive or negative controls or other antigens known to bind to autoantibodies of diagnostic value, particularly those related to other APS1 associated conditions. For example, the panel can include one or more polypeptides or fragments thereof of any of the following previously identified APS1 autoantigen: IL22, IFNAβ, IFNA2, IFNA5, IL17A, IFNA13, CYP21A2, CYP17A1, CYP11A1, MAGEB1, MAGEB10, MAGEB4, NLRP5, KCNRG, PDILT, IL17F, SPAG16, LCN1, DDC, CYP1A2, GGA1, SYNPR, SOX9, SOX10, or ZNF41D.

In one aspect, provided herein is a medical or diagnostic device comprising a panel as described above, the panel including one or more APS1 autoantigen polypeptides listed in Table 1 or antigenic fragments or variants thereof. In some embodiments, such a medical or diagnostic panel device comprises the one or more APS1 autoantigen polypeptides, fragments, or variants in a form as described above that allows contacting it with an aqueous solution, more preferably the liquid human sample, in a straightforward manner. In particular, the one or more APS1 autoantigen polypeptides, fragments, or variants may be immobilized on the surface of a carrier, which carrier comprises, but is not limited to glass plates or slides, biochips, microtiter plates, beads, for example magnetic beads, chromatography columns, membranes or the like. Exemplary devices include line blots, microtiter plates and biochips. In some embodiments, the device can include additional polypeptides such as, for example, positive or negative controls or other antigens known to bind to autoantibodies of diagnostic value, particularly those related other neurological diseases, including neurodegenerative disease and paraneoplastic neurologic syndromes as discussed above. In some embodiments, the APS1 autoantigen polypeptides or antigenic fragments or variants thereof include one or more of a RFX6 polypeptide or antigenic fragment or variant. In some embodiments, the APS1 autoantigen polypeptides or antigenic fragments or variants thereof include one or more of a KHDC3L polypeptide or antigenic fragment or variant. In some embodiments, the APS1 autoantigen polypeptides or antigenic fragments or variants thereof include one or more of an ACPT polypeptide or antigenic fragment or variant. In some embodiments, the APS1 autoantigen polypeptides or antigenic fragments or variants thereof include one or more of a PLIN1 polypeptide or antigenic fragment or variant.

### C. Medical Methods

In one aspect, provided are methods of diagnosing a subject having APS1. The methods comprise detecting the presence of APS1-specific autoantibodies in a biological sample from the subject using an in vitro detection method, particularly methods using immunohistochemical detection of the APS1-specific autoantibodies. In some embodiments, where a subject that is determined to be positive for an APS1-specific autoantibody (i.e. the APS1-specific autoantibody is detected in the sample from the subject), the subject is diagnosed as having APS1.

In another aspect, provided are methods of identifying a subject that is at risk of developing an APS1 associated condition. In some embodiments, where a subject that is determined to be positive for an APS1-specific autoantibody (i.e. the APS1-specific autoantibody is detected in the sample from the subject), the subject is determined to be at risk of developing an APS1 associated condition. In some instances, the APS1 associated condition is primary ovarian insufficiency. In some instances, the APS1 associated condition is intestinal dysfunction, particularly diarrheal-type intestinal dysfunction.

In another aspect, provided are methods of treating a subject having APS1, wherein the subject produces one or more APS1-specific autoantibodies (i.e. an APS1-specific autoantibody is present in at least type of biological sample from the subject).

In another aspect, provided are methods of treating a subject that is at risk of developing an APS1 associated condition, wherein the subject produces one or more APS1-specific autoantibodies (i.e. an APS1-specific autoantibody is present in at least type of biological sample from the subject). In some embodiments, provided is a method of treating a subject that is at risk of developing diarrheal-type intestinal dysfunction. In some embodiments, provided is a method of treating a subject that is at risk of developing primary ovarian insufficiency.

The provided methods include a step of detecting the presence or absence of an APS1-specific autoantibody that binds specifically to an autogenic protein listed in Table 1 in a biological sample from the subject. In some embodiments, the subject is seropositive for at least one APS1-specific autoantibody provided in this disclosure. In some embodiments, an APS1-specific autoantibody detected in the provided methods may be a RFX6 autoantibody. In some embodiments, an APS1-specific autoantibody detected in the provided methods may be a KHDC3L autoantibody. In some embodiments, an APS1-specific autoantibody detected in the provided methods may be an ACPT autoantibody. In some embodiments, an APS1-specific autoantibody detected in the provided methods may be a PLIN1 autoantibody.

In some instances, the provided methods can include a step of performing an examination of the subject to determine the presence of an APS1 associated condition in the subject when the subject is determined to express an APS1-specific autoantibody that is detected according to the provided methods. In some instances, the subject is assessed to determine whether the subject has a loss of function mutation of the AIRE gene. Genetic testing of a DNA sample from a subject is routine and can include one or more of assessment by polymerase chain reaction (PCR), DNA sequencing, cytogenetics, microarray analysis, or gene expression profiling.

The provided methods of treatment include a step of administering an immunosuppressive therapy to the subject. In some embodiments, the subject is determined to have an APS1 associated condition and is also administered a treatment for the APS1 associated condition.

The provided methods are of use for subjects having APS1 as discussed above in Section A. Any of the detection methods, kits, or devices discussed above in Sections A and B may be used to detect an APS1-specific autoantibody in a biological sample from the subject. In some instances, the subject may express more than one APS1 autoantibody. For example, the subject may express autoantibodies that specifically bind to one or more of the autoantigen proteins listed in Table 1. In some embodiments, the APS1-specific autoantibody can be a RFX6 autoantibody. In some embodiments, the APS1-specific autoantibody can be a KHDC3L autoantibody. In some embodiments, the APS1-specific autoantibody can be an ACPT autoantibody. In some embodiments, the APS1-specific autoantibody can be a PLIN1 autoantibody. In some embodiments, the subject is determined to be serologically positive for an APS1-specific autoantibody that binds to one of the autoantigens listed in Table 1. The treatment administered to the subject can be any of the treatments discussed below or are otherwise appropriate for the type and/or stage of APS1.

In some embodiments, as discussed in detail elsewhere in this disclosure, the subject has one or more of nail dystrophy, hypoparathyroidism, keratoconjunctivitis, chronic mucocutaneous candidiasis, intestinal dysfunction, autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis. In some instances, where the subject being treated is at risk for developing a particular APS1 associated condition, the subject does not yet exhibit the symptoms of such condition or may exhibit only early signs indicative of the condition.

In some embodiments, the subject has not been previously diagnosed or treated for APS1. In such instances the provided methods are useful for directing a focused assessment of the subject for APS1 associated conditions.

In some embodiments, the subject was previously treated for APS1. In such instances the provided methods are useful for investigating symptoms that may be indicative of APS1 associated conditions. The provided methods are also useful for detecting early evidence of symptoms indicative of APS1 associated conditions in patients that previously were determined not to be positive for the APS1-specific autoantibodies provided herein. In some embodiments, the subject can be assessed for the presence of a RFX6 autoantibody to determine the risk of developing diarrheal-type intestinal dysfunction. In some embodiments, the subject can be assessed for the presence of a KHDC3L autoantibody to determine the risk of developing diarrheal-type intestinal dysfunction. In some embodiments, the subject can be assessed for the presence of an ACPT autoantibody to determine the risk of developing dental enamel hypoplasia. In some embodiments, the subject can be assessed for the presence of a PLIN1 autoantibody to determine the risk of developing lipodystrophy. In some instances, the subject can be assessed for the presence of an autoantibody that specifically binds to one of the autoantigen proteins listed in Table 1 to determine the risk of developing an APS1 associated condition identified in FIG. 5, which identifies conditions associated with the autoantigen proteins listed in Table 1 as well as other known APS1 autoantigens.

In some aspects, methods provided herein may be used to assess the presence of an ACPT autoantibody in a subject that does not have or is not suspected to have APS1. In some aspects, methods provided herein may be used to assess the presence of an ACPT autoantibody in a subject that has dental enamel hypoplasia. In some aspects, the presence of ACPT autoantibodies in a biological sample may be indicative for the presence or the risk to develop dental enamel hypoplasia that is not associated with or caused by APS1. In some aspects, the presence of ACPT autoantibodies in a biological sample may be indicative of dental enamel hypoplasia that is associated with another autoimmune condition.

According to some embodiments, provided are methods of identifying a subject that is at risk of developing dental enamel hypoplasia by detecting in a biological sample the presence of ACPT autoantibodies. In some aspect, the subject has an autoimmune condition. In some embodiments, the subject has type II diabetes. In some embodiments, the subject has irritable bowel syndrome (IBS). In some aspects, the subject has a tooth related condition and is suspected to have dental enamel hypoplasia. In some aspects, the subject has type II diabetes and is suspected to have dental enamel hypoplasia. In some aspects, the subject has IBS and is suspected to have dental enamel hypoplasia. In some embodiments, the subject can be assessed for the presence of an ACPT autoantibody to determine the risk of developing dental enamel hypoplasia. In some approaches, a biological sample is obtained from the subject and the presence of the binding of the ACPT antigenic polypeptide or fragment thereof to the ACPT autoantibody in the biological sample indicates that the subject is at risk of developing dental enamel hypoplasia. In some embodiments, the subject has dental enamel hypoplasia, and the presence or absence of an ACPT autoantibody is detected in the biological sample. Also provided is a method of treating a subject that is at risk of developing dental enamel hypoplasia, comprising (a) detecting the presence or absence of an ACPT autoantibody in a biological sample from a subject using the methods described herein, where detecting the autoantibodies in the biological sample indicates that the subject is at risk of developing dental enamel hypoplasia, and (b) administering to the subject therapy for dental enamel hypoplasia or an immunosuppressive therapy as described below.

In the provided methods of treatment, an immunosuppressive therapy is administered to the subject to treat APS1. Immunosuppressive therapies are therapies that lower the activity the body's immune system. Such therapies are useful to treat conditions in which the immune system is overactive, such as autoimmune diseases (e.g., paraneoplastic encephalomyelitis). The immunosuppressive therapy administered to the subject includes at least one of an immunosuppressant drug, intravenous immunoglobulin administration, plasma exchange plasmapheresis, or immunoadsorption. A discussion of suitable therapies is provided by Shin, Y-W., et al., 2018 Ther. Adv. Neurol. Disord. 11: 1756285617722347, published online Aug. 26, 2017 (doi: 10.1177/1756285617722347). For example, first-line immunosuppressive therapy can include corticosteroids, intravenous immunoglobulin, plasma exchange plasmapheresis, and immunoadsorption. In some instances, second-line immunosuppressive therapy may be need to be administered. Exemplary second-line immunosuppressive therapy is treatment with immunosuppressant drugs such as rituximab and cyclophosphamide.

Immunosuppressant drugs include corticosteroids (such as prednisone, budesonide, prednisolone), Janus kinase inhibitors (such as tofacitinib), calcineurin inhibitors (such as cyclosporine, tacrolimus), mTOR inhibitors (such as sirolimus, everolimus), IMDH inhibitors (such as azathioprine, lefunomide, mycophenolate), monoclonal antibodies (such as basiliximab, daclizumab, muromonab, adalimumab, certolizumab, etanercept, golimumab, infliximab, ixekizumab, natalizumab, rituximab, secukinumab, tocilizumab, ustekinumab, vedolizumab), and other biologics (such as abatacept, anakinra). Therapeutic targets for these immunosuppressant drugs include B cells and short-lived plasma cells (rituximab) and specific cytokines associated in the autoimmune and inflammatory process (tocilizumab and low-dose interleukin (IL)-2). Antiproliferative agents targeting lymphocyte proliferation (cyclophosphamide, azathioprine, mycophenolate mofetil, etc.) can also be used in refractory cases or to maintain remission.

In some instances, the subject is administered corticosteroids. Corticosteroids act to broadly inhibit the inflammatory process and possess less specificity for the antibody-mediated immune process. Corticosteroids bind to intracellular glucocorticoid receptors and suppress the transcription of multiple proinflammatory genes that encode cytokines, chemokines, adhesion molecules, inflammatory enzymes, receptors and proteins. Glucocorticoids have an influence on almost all cytokines, and their use results in the depletion of T cells, inhibition of Th1 differentiation, macrophage dysfunction, and eosinophil apoptosis. At higher concentration, corticosteroids have additional effects on the synthesis of anti-inflammatory proteins, and also induce post-transcriptional effects. Furthermore, corticosteroids offer extra benefit to CNS inflammatory disorders by restoring BBB integrity and controlling brain edema.

Intravenous immunoglobulin (IVIG) is a blood product extracted from the collected pool of plasma from over a at least 25 to over a thousand donors. IVIG provides antibodies to a broad range of pathogens and is used to provide passive immunity for patients with immunodeficiency. High-dose IVIG (1-2 g/kg) provide various anti-inflammatory and immunomodulatory effects by multidirectional mechanisms such as autoantibody neutralization, blockade of activating FcγR, and upregulation of inhibitory FcγRIIB, inhibition of complements, cytokines, and leukocyte migration. In some instances, IVIg is administered as a monotherapy. In some instances, after or in combination with high-dose steroids, or with plasma exchange plasmapheresis, rituximab, or other immunotherapeutic agents.

Plasma exchange plasmapheresis (also referred to as PLEX) removes autoantibodies and other pathologic substances in the plasma. PLEX also alters the immune system by changing lymphocyte numbers and their distribution, T-suppressor cell function, and T-helper cell phenotypes. Steroids alone can be insufficient to ameliorate autoantibody-mediated immune process, and direct removal or neutralization of autoantibodies from the circulation by PLEX and IVIg may show a synergistic effect. In addition, PLEX increases the proliferation of antibody-producing cells and this could increase susceptibility of these cells to immunosuppressants and chemotherapeutic agents. Immunoadsorption is a refined form of PLEX that enables the selective removal of Igs from separated plasma through high-affinity adsorbers (e.g., Protein A).

In some instances, the subject can be treated with an immunosuppressive therapy comprising oral administration of KLHL11 polypeptide or immunogenic fragments thereof. Such therapy is termed oral tolerization and involves treatment of by feeding to the subject the autoantigen inducing the immune response in order to suppress the immune response by invoking oral tolerance.

In some embodiments, the provided methods of treatment include the step of administering a treatment for an APS1 associated condition to the subject according to the APS1 autoantibody detected in the sample from the subject. In some instances, when the subject is determined to be positive for an RFX6 autoantibody, the treatment for the APS1 associated condition can be a treatment for diarrheal-type intestinal dysfunction. In some instances, when the subject is determined to be positive for an KHDC3L autoantibody, the treatment for the APS1 associated condition can be a treatment for primary ovarian insufficiency. In some instances, when the subject is determined to be positive for an ACPT autoantibody, the treatment for the APS1 associated condition can be a treatment for dental enamel hypoplasia. In some instances, when the subject is determined to be positive for a PLIN1 autoantibody, the treatment for the APS1 associated condition can be a treatment for lipodystrophy.

In some instances, treatment of the subject includes reducing the amount of APS1-specific autoantibodies in the subject such as, for example RXF6 autoantibodies, KHDC3L autoantibodies, ACPT autoantibodies, PLIN1 autoantibodies, or any combination thereof. For example, the amount of APS1-specific autoantibodies may be reduced by plasmapheresis, which involves the removal of blood from the subject, separation of the plasma from blood cells, and reinjection of the cells. In particular embodiments, plasmapheresis may be carried out by plasma exchange, in which the plasma removed is replaced in whole or in part by a plasma substitute such as lactated Ringer's solution, or by plasma perfusion, in which the autoantibodies are separated from the plasma and the plasma then returned to the subject.

Autoantibodies may be removed from the subject's plasma by any suitable technique, such as by contacting the plasma to a solid support having an immunoglobulin binding protein (e.g., protein A) immobilized thereon), removing the plasma from the solid support, and then returning the plasma to the subject. Typically, this method is practiced by immobilizing the immunoglobulin binding protein on an affinity support in an affinity column, passing the blood plasma through the affinity column, and returning the plasma to the subject (optionally, but preferably, recombining the plasma with the patient's blood cells). The method may also be carried out on whole blood or other suitable blood fraction, which may then be recombined with blood cells and then returned to the patient, as will be appreciated by persons skilled in the art.

In a variation of the foregoing, the APS1 autoantigen polypeptide listed in Table 1 or one or more antigenic fragments or variants thereof may be immobilized on the solid support, and the APS1-specific autoantibodies selectively removed from the subject's blood by contacting the subject's blood, blood plasma, or other suitable fraction to the solid support, as described above. Such procedures advantageously avoid substantial reduction in levels of other antibodies in the subject undergoing treatment. In some embodiments, one or more RXF6 polypeptides or antigenic fragments or variants thereof may be immobilized on a solid support to remove RXF6 autoantibodies from the subject. In some embodiments, one or more KHDC3L polypeptides or antigenic fragments or variants thereof may be immobilized on a solid support to remove KHDC3L autoantibodies from the subject. In some embodiments, one or more ACPT polypeptides or antigenic fragments or variants thereof may be immobilized on a solid support to remove ACPT autoantibodies from the subject. In some embodiments, one or more PLIN1 polypeptides or antigenic fragments or variants thereof may be immobilized on a solid support to remove PLIN1 autoantibodies from the subject.

In some instances, following treatment, the subject can be monitored at regular intervals over time (such as bi-monthly, quarterly, bi-annually, annually, or every two to five years) for indications of APS1 associated conditions

In some instances, the subject is monitored during the course of or after treatment using the provided methods. In some instances, the provided methods can be used to monitor the immune response of a subject that has been determined to have elevated APS1-specific autoantibodies during the course of treatment. For example, if therapy is successful in treating the subject's APS1, the provided methods should reflect a reduction or elimination of detectable the provided APS1-specific autoantibodies in a biological sample from the subject. If APS1-specific autoantibodies remain detectable in a biological sample during or after therapy, additional treatment may be necessary. In some instances, the provided methods can be used to detect early evidence of the development of an APS1 associated condition in a subject that has been determined to have detectable APS1-specific autoantibodies. Following successful treatment, the provided methods can be used during continued monitoring of the subject at regular intervals over time to assess APS1-specific autoantibody levels. In some instances, if the one or more of the provided APS1-specific autoantibodies are detected using the provided methods, the subject can have recurrence of an APS1 associated condition.

In another aspect, provided are methods of imaging APS1 autoantigen-expressing cells in a subject. The method comprises administering to the subject an effective amount of an antibody having specific binding affinity for an APS1 autoantigen listed in Table 1 that is labeled with an imaging agent under conditions in which the antibody binds to a APS1 autoantigen released from, or accessible in, cells, and detecting any complex so formed. The antibody can be labeled either directly or indirectly, and a wide variety of labels, including radioactive labels (radioisotopes), enzymes, substrates, cofactors, inhibitors, fluorescers, chemiluminescers, magnetic particles, and imaging agents. Exemplary imaging agents include radioactive labels and fluorescent labels (chemical moiety or heterologous polypeptide) as described above and heavy metal labels (such as, for example ¹¹¹In, ⁹⁷Ru, ⁶⁷Ga, ⁶⁸Ga, ⁷²As, ⁸⁹Zr, and ²⁰¹Tl). Techniques for conjugation of imaging agents and other labels are known and are reported extensively in both the scientific and patent literature. As is well known to those of ordinary skill in the art, a suitable amount of an antibody for this method of detection is any amount that is effective to image cells, for example, with regards to a radioactive label, about 0.1 mCi to about 50.0 mCi. In addition, an effective amount of an antibody to detect an APS1 autoantigen may be an amount from about 0.01 mg to about 100 mg. Suitable methods of administering the imaging agent are as described above and can be targeted as described above. Methods of imaging are dependent upon the agent used and are well known to those of skill in this art.

In accordance with the present disclosure, there may be employed conventional molecular biology, microbiology, biochemical, and recombinant DNA techniques within the skill of the art. Such techniques are explained fully in the literature. The provided methods and compositions will be further described in the following examples, which do not limit the scope of the methods and compositions of matter described in the claims.

### EXAMPLES

### Example 1. Identification and Characterization of APS1 Antigens: Approach & Methods

The experiments described in Examples 1-10 are also described in Vasquez, S. E. et al., eLife 9:e55053 (2020), doi: 10.7554/eLife.55053, which is incorporated by reference in its entirety for all purposes.

A programmable phage display based on Phage Immunoprecipitation-Sequencing (PhIP-seq) using an established proteome-wide tiled library was used to identify APS1 antigens. This approach has been described previously by Larman et al., 2011 and O'Donovan et al., 2018, as well as in WO 2020/190700), which is incorporated by reference in its entirety for all purposes. This approach possesses many advantages over previous candidate-based and whole-protein fixed array approaches, including (1) expanded, proteome-wide coverage (including alternative splice forms) with 49 amino acid (AA) peptide length and 24AA resolution tiling, (2) reduced volume requirement for patient serum, and (3) high-throughput, sequencing based output (Larman et al., 2011; O'Donovan et al., 2018). Of note, the PhIP-seq investigation of autoimmune diseases of the central nervous system, including paraneoplastic disease, has yielded novel and specific biomarkers of disease (Larman et al., 2011; Mandel-Brehm et al., 2019; O'Donovan et al., 2018). It was reasoned that PhIP-seq interrogation of APS1, a defined monogenic autoimmune syndrome with a broad spectrum of high affinity autoantibodies, would likely yield clinically meaningful targets—consistent with the clinical utility derived from previous APS1 autoantigen identification efforts (Shum et al., 2013; Velloso et al., 1994).

As described in the following examples, using a PhIP-seq autoantibody survey, a plethora of novel APS1 autoantigens were identified as well as numerous known, literature-reported APS1 autoantigens. The novel autoantigens which were orthogonally validated include RFX6, KHDC3L, and ACPT, all of which exhibit tissue-restricted expression (Patel et al., 2017; Rezaei et al., 2016; Seymen et al., 2016; C. E. Smith et al., 2017; S. B. Smith et al., 2010; K. Zhu et al., 2015). Importantly, these novel autoantigens may carry important implications for poorly understood clinical manifestations such as intestinal dysfunction, ovarian insufficiency, and tooth enamel hypoplasia where underlying cell-type specific candidate antigens have remained elusive (Bruserud et al., 2016; Ferré et al., 2019; Ferré et al., 2016; Sorkina et al., 2016). Together, these results demonstrate the applicability of PhIP-seq to antigen discovery, substantially expand the spectrum of known antibody targets and clinical associations in APS1, and point towards novel tissue and cell type specificities that can be targeted in the setting of APS1 autoimmunity. The following materials and methods were used in these examples.

Data Collection: All patient cohort data were collected and evaluated at the NIH, and all APECED patients were enrolled in research study protocols approved by the NIAID, NIH Clinical Center, and NCI Institutional Review Board Committee and provided with written informed consent for study participation. All NIH patients gave consent for passive use of their medical record for research purposes (protocol #11-I-0187). The majority of these cohort data were previously published by Ferré et al. 2016 and Ferré et al. 2019.

Phage Immunoprecipitation—Sequencing (PhIP-seq): For PhIP-seq, a custom-designed phage library consisting of 731,724 49AA peptides tiling the full protein-coding human genome including all isoforms (as of 2016) with 25AA overlap as previously described (O'Donovan et al., 2018) was used. One milliliter of phage library was incubated with 1 μL of human serum overnight at 4 degrees Celsius, and patient antibody (bound to phage) was immunoprecipitated using 40 μL of a 1:1 mix of protein A/G magnetic beads (Thermo Fisher, Waltham, Mass., #10008D & #10009D). Beads were washed 4 times and antibody-bound phage were eluted into 1 mL of E. Coli at OD of 0.5-0.7 (BLT5403, EMD Millipore, Burlington, Mass.) for selective amplification of eluted phage. This library was re-incubated with patient serum and the above steps were repeated (total of two immunoprecipitations), followed by phenol-chloroform extraction of DNA from the final phage library. DNA was barcoded and amplified (Phusion PCR, 30 rounds), gel purified, and subjected to next-generation sequencing on an Illumina MiSeq Instrument (Illumina, San Diego, Calif.).

PhIP-seq Analysis: Sequencing reads from fastq files were aligned to the reference oligonucleotide library and peptide counts were subsequently normalized by converting raw reads to percentage of total reads per sample. Peptide and gene-level enrichments for both patient and healthy control sera were calculated by determining the fold-change of read percentage per peptide and gene in each sample over the mean read percentage per peptide and gene in a background of mock-IP (A/G bead only, n=17). Individual patients and controls were considered ‘positive’ for genes where the enrichment value was 10-fold or greater as compared to mock-IP. For plotting of multiple genes in parallel, enrichment values were z-scored and hierarchically clustered using Pearson correlation.

Statistics: For comparison of distribution of PhIP-seq gene enrichment between APS1 patients with and without specific disease manifestations, a (non-parametric) Kolmogorov-Smirnov test was used. For radioligand binding assays, antibody index for each sample was calculated as follows: (sample value−mean blank value)/(positive control antibody value−mean blank value). Comparison of antibody index values between healthy controls and APS1 samples was performed using a 2-tailed unpaired t test. Experimental samples that fell 3 standard deviations above the mean of healthy controls for each assay were considered positive, except in the case of RFX6, where a cutoff of 6 standard deviations above the mean of healthy controls was used.

Assessing Tissue-Specific RNA Expression: To determine tissue-specificity and tissue-restriction of Rfx6 expression in mice, publicly available Tabula Muris data were used (Schaum et al., 2018). For investigation of KHDC3L expression in human ovary, publicly available normalized FPKM transcriptome data from human oocytes and granulosa cells were downloaded (GSE107746 Folliculogenesis FPKM.log 2.txt) (Zhang et al., 2018). Principle component analysis clustered the two cell types correctly according to their corresponding sample label, and log 2(FPKM) values were plotted by color for each sample.

293T Overexpression Assays: Human embryonic kidney 293T cells (ATCC, Manassas, Va., #CRL-3216) were plated at 30% density in a standard 24-well glass bottom plate in complete Dulbecco's Modified Eagle Medium (Thermo Fisher, #119651198) with 10% Fetal Bovine Serum (Thermo Fisher, #10438026), 292 μg/mL L-glutamine, 100 μg/mL Streptomycin Sulfate, and 120 Units/mL of Penicillin G Sodium (Thermo Fisher, #10378016). Eighteen hours later, cells were transiently transfected using a standard calcium chloride transfection protocol. For transfections, 0.1 μg of sequence-verified pCMV-insert-MYC-FLAG overexpression vectors containing either no insert (Origene #PS100001; ‘mock’ transfection) or RFX6 insert (Origene #RC206174) were transfected into each well. Twenty-four hours post-transfection, cells were washed in 1×PBS and fixed in 4% PFA for 10 minutes at room temperature.

293T Indirect Immunofluorescence: Fixed 293T cells were blocked for 1 hour at room temperature in 5% BSA in PBST. For primary antibody incubation, cells were incubated with human serum (1:1000) and rabbit anti-FLAG antibody (1:2000) in 5% BSA in PBST for 2 hours at room temperature. Cells were washed 4 times in PBST and subsequently incubated with secondary antibodies (goat anti-rabbit IgG 488, Invitrogen, Carlsbad, Calif.; #A-11034, 1:4000; & goat anti-human 647, Invitrogen #A-21445, 1:4000) for 1 hour at room temperature. Finally, cells were washed 4 times in PBST, incubated with DAPI for 5 minutes at room temperature, and subsequently placed into PBS for immediate imaging. All images were acquired with a Nikon Ti inverted fluorescence microscope.

Indirect Dual Immunofluorescence On Human Fetal Intestine: Human fetal small bowels (21.2 days gestational age) were processed as previously described (Berger et al., 2015). Individual sera from APS1 patients (1:4000 dilution) were used in combination with rabbit antibodies to human Chromogranin A (Abcam, Cambridge, Mass.; #ab15160, 1:5000 dilution). Immunofluorescence detection utilized Alexa Fluor secondary antibodies (Life Technologies, Waltham, Mass.; 488 goat anti-human IgG, #A11013; and 546 goat anti-rabbit IgG, #A11010). Nuclear DNA was stained with Hoechst dye (Invitrogen, #33342). All images were acquired with a Leica SP5 White Light confocal laser microscope.

35S-Radiolabeled Protein Generation and Binding Assay (RLBA): DNA plasmids containing full-length cDNA under the control of a T7 promoter for each of the validated antigens (see Table 3 below) were verified by Sanger sequencing and used as DNA templates in the T7 TNT in vitro transcription/translation kit (Promega, Madison, Wis.; #L1170) using [³⁵S]-methionine (PerkinElmer, Waltham, Mass.; #NEG709A). Protein was column-purified on Nap-5 columns (GE healthcare, Chicago, Ill.; #17-0853-01) and immunoprecipitated on Sephadex protein A/G beads (Sigma Aldrich, St. Louis, Mo.; #GE17-5280-02 and #GE17-0618-05, 4:1 ratio) in duplicate with serum or control antibodies in 96-well polyvinylidene difluoride filtration plates (Corning, Corning, N.Y.; #EK-680860). Each well contained 35,000 counts per minute (cpm) of radiolabeled protein and 2.5 μL of serum or appropriately diluted control antibody (see Table 3 below). The cpm of immunoprecipitated protein was quantified using a 96-well Microbeta Trilux liquid scintillation plate reader (Perkin Elmer).

TABLE 3
Antibody information by application.

| Antibody | Assay Use* | Dilution |
|---|---|---|
| Anti-NLRP5 (Santa Cruz, Dallas, TX; #sc-50630) | NLRP5 RLBA | 1:50 |
| Anti-SOX10 (Abcam, Cambridge, MA, #ab181466) | SOX10 RLBA | 1:25 |
| Anti-RFX6 (R&D Systems, Minneapolis, MN; | RFX6 RLBA | 1:50 |
| #AF7780) | | |
| Anti-KHDC3L (Abcam, #ab170298) | KHDC3L RLBA | 1:25 |
| Anti-CYP11A1 (Abcam, #ab175408) | CYP11A1 RLBA | 1:50 |
| Anti-NKX6-3 (Biorbyt, Cambridge, | NKX6-3 RLBA | 1:50 |
| Cambridgeshire, UK; #orb127108) | | |
| Anti-GIP (Abcam, #ab30679) | GIP RLBA | 1:50 |
| Anti-PDX1 (Invitrogen, Carlsbad, CA, #PA5- | PDX1 RLBA | 1:50 |
| 78024) | | |
| Anti-ASMT (Invitrogen, #PA5-24721) | ASMT RLBA | 1:25 |
| Anti-CHGA (Abcam, Cambridge, MA, USA, | Tissue IF | 1:5000 |
| #ab15160) | | |
| Human serum | Tissue IF | 1:4000 (Tissue) |
| | CBA IF | 1:500 (CBA) |
| | RLBA | 1:25 (RLBA) |
| Secondary abs: | | |
| 488 goat anti-human IgG (Life Technologies, | Tissue IF | 1:400 |
| Waltham, MA, USA: #A11013) | | |
| 546 goat anti-rabbit IgG (Life Technologies, | | |
| A11010) | | |
| Secondary abs: | | |
| 647 goat anti-human IgG (Thermo Fisher, #A- | CBA IF | 1:1000 |
| 21445) | | |
| 488 goat anti-rabbit IgG (Thermo Fisher, #A- | | |
| 11034) | | |
| Anti-DYKDDDDK (D6W5B) (Cell Signaling | CBA IF; | 1:2000 (CBA IF) |
| Technologies, Danvers, MA; #14793) | ACPT RLBA | 1:125 (RLBA) |
| Nuclear staining: | | |
| Hoechst dye (Invitrogen, #33342) | Tissue IF | |
| DAPI (Thermo Fisher, #D1306) | CBA IF | |

*IF: immunofluorescence; RLBA: radioligand binding assay; CBA: cell-based assay

### Example 2. PhIP-Seq Investigation of APS1 Patient Cohort

Patients with APS-1 develop autoantibodies to many known protein targets, some of which exhibit tissue-restricted expression and have been shown to correlate with specific autoimmune disease manifestations. However, the target proteins for many of the APS1 tissue-specific manifestations remain enigmatic. To this end, a high-throughput, proteome-wide programmable phage display approach (PhIP-seq) was used to query the antibody target identities within serum of patients with APS1 (Larman et al., 2011; O'Donovan et al., 2018). The PhIP-seq technique leverages large scale oligo production and efficient phage packaging and expression to present a tiled-peptide representation of the proteome displayed on T7 phage. Here, a phage library that was previously designed and deployed for investigating paraneoplastic autoimmune encephalitis (O'Donovan et al., 2018) was utilized. The library itself contains approximately 700,000 unique phage, each displaying a 49 amino acid proteome segment. Phage were immunoprecipitated using patient antibodies bound to protein A/G beads. In order to increase sensitivity and specificity for target proteins, eluted phage were used for a further round of amplification and immunoprecipitation. DNA was then extracted from the final phage elution, amplified and barcoded, and subjected to Next-Generation sequencing. Finally, sequencing counts were normalized across samples to correct for variability in sequencing depth, and the fold-change of each gene was calculated (comprised of multiple unique tiling phage) as compared to mock IPs in the absence of human serum. A complete protocol is detailed in Example 1.

From a cohort of 67 APS1 patients, a total of 39 serum samples were subjected to PhIP-seq investigation, while the remaining 28 samples were obtained at a later time point and reserved for downstream validation experiments (for clinical data, see Table 4 below). In addition, 28 healthy anonymous blood donor sera samples were subjected to PhIP-Seq, and an additional group of 61 healthy donor sera were used for downstream validation experiments. Putative gene-level target antigens were considered to be “positive” by this assay if their normalized peptide counts were a minimum of 10-fold greater than the mean derived from 17 mock IPs (beads, no sera) in at least 2 out of 39 APS1 patient samples. In addition, the requirement was set that none of the 28 healthy control sera subjected to PhIP-Seq exhibited signal above the same threshold for the same target. For visualization purposes, z-scored enrichments were used.

TABLE 4
APS1 Cohort: Clinical Data.

| Patient | | | | |
|---|---|---|---|---|
| Code | Gender | Age* | Clinical Phenotypes | Cohort |
| AIRE.04 | F | 14 | CMC, HP, AI, DM, EH, ND, HTN, SS, Pneumonitis, | D |
| | | | Urticarial eruption, GH def, ID (D) | |
| AIRE.05 | F | 11 | CMC, HP, AI, AIH, Gastritis, EH, HTN, Pneumonitis, | D |
| | | | Urticarial eruption, Vitiligo, ID (B) | |
| AIRE.09 | F | 10 | HP, AIH, EH, Pneumonitis, Urticarial eruption | D |
| AIRE.13 | F | 10 | CMC, HP, AI, Gastritis, Urticarial eruption, Vitiligo, ID (D) | D |
| AIRE.14 | M | 7 | CMC, AI, AIH, DM, Gastritis, EH, ND, KC, SS, | D |
| | | | Pneumonitis, Urticarial eruption, Vitiligo, B12 def, ID (C) | |
| AIRE.18 | F | 18 | CMC, HP, AI, POI, ND, SS | D |
| AIRE.19 | M | 12 | CMC, HP, AI, AIH, Gastritis, EH, Pneumonitis, Urticarial | D |
| | | | eruption, GH def, Asplenia, ID (B) | |
| AIRE.20 | F | 25 | CMC, AI, Gastritis, EH, KC, SS, ID (D) | D |
| AIRE.21 | M | 65 | CMC, HP, AI, HT, DM, EH, HTN, SS, Vitiligo, B12 def, | D |
| | | | ID (B) | |
| AIRE.23 | M | 38 | CMC, HP, AI, Gastritis, TIN, EH, ND, KC, HTN, Vitiligo, | D |
| | | | Alopecia, B12 def, Asplenia, ID (D) | |
| AIRE.24 | F | 15 | CMC, HP, AI, AIH, Gastritis, EH, KC, ID (C) | D |
| AIRE.27 | M | 18 | CMC, AI, AIH, DM, Gastritis, EH, KC, SS, Pneumonitis, | D |
| | | | Urticarial eruption, Vitiligo, B12 def, ID (B) | |
| AIRE.08 | M | 11 | CMC, HP, AI, EH, Urticarial eruption | D |
| AIRE.07 | M | 12 | CMC, HP, AI, HT, ND, KC, Alopecia, ID (C) | D |
| AIRE.28 | M | 15 | CMC, HP, AI, Gastritis, EH, KC, Vitiligo, ID (C) | D |
| AIRE.22 | F | 7 | CMC, HP, AIH, Gastritis, EH, ND, SS, | D |
| | | | Pneumonitis, Urticarial eruption, Alopecia, GH def, ID (D) | |
| AIRE.29 | M | 9 | AI, AIH, EH, ND, Urticarial eruption, Vitiligo, Alopecia, | D |
| | | | ID (B) | |
| AIRE.30 | M | 17 | CMC, HP, AIH, EH, Urticarial eruption, Vitiligo, Alopecia, | D |
| | | | B12 def, ID (C) | |
| AIRE.23c | F | 41 | CMC, HP, AI, HT, POI, EH, SS | D |
| AIRE.31 | F | 18 | CMC, HP, AI, AIH, DM, EH, ND, KC, SS, Urticarial | D |
| | | | eruption, Vitiligo, ID (D) | |
| AIRE.33 | F | 14 | HP, AI, AIH, POI, EH, Urticarial eruption, ID (D) | D |
| AIRE.34 | F | 54 | CMC, HP, AI, HT, POI, Gastritis, EH, HTN, SS, | D |
| | | | Pneumonitis, B12 def, ID (D) | |
| AIRE.35 | F | 23 | CMC, HP, AI, AIH, HT, POI, Gastritis, EH, SS, | D |
| | | | Pneumonitis, Urticarial eruption, B12 def, Asplenia, ID (D) | |
| AIRE.11 | M | 19 | CMC, HP, AI, AIH, TF, Gastritis, EH, SS, Urticarial | D |
| | | | eruption, Vitiligo, GH def, ID (B) | |
| AIRE.36 | M | 15 | HP, AI, EH, Alopecia | D |
| AIRE.37 | F | 28 | CMC, HP, AI, POI, EH, SS, Urticarial eruption, ID (D) | D |
| AIRE.38 | F | 7 | HP, AI, EH, ND, Urticarial eruption, Alopecia, ID (C) | D |
| AIRE.17 | F | 6 | CMC, HP, EH, KC, Urticarial eruption, ID (D) | D |
| AIRE.39 | F | 18 | CMC, HP, AI, AIH, HT, EH, ND, KC, Pneumonitis, | D |
| | | | Urticarial eruption, ID (B) | |
| AIRE.40 | F | 16 | CMC, HP, AI, AIH, POI, EH, Pneumonitis, Urticarial | D |
| | | | eruption, Alopecia, Asplenia, ID (D) | |
| AIRE.41 | M | 20 | CMC, AI, HT, TF, EH, HTN, Vitiligo, Alopecia, ID (D) | D |
| AIRE.44 | F | 24 | CMC, HP, AI, POI, Gastritis, EH, ND, KC, HTN, SS, | D |
| | | | Urticarial eruption, Alopecia, B12 def, ID (D) | |
| AIRE.46 | F | 22 | CMC, HP, AI, EH, KC, SS, B12 def, GH def, ID (B) | D |
| AIRE.12 | F | 7 | CMC, HP, AI, Gastritis, EH, KC, SS, Pneumonitis, | D |
| | | | Urticarial eruption, Vitiligo, B12 def, ID (D) | |
| AIRE.06 | F | 16 | CMC, HP, AI, AIH, HT, Gastritis, EH, HTN, SS, Vitiligo, | D |
| | | | ID (D) | |
| AIRE.50 | F | 26 | CMC, AI, Gastritis, HTN, SS, Pneumonitis, Urticarial | D |
| | | | eruption, B12 def, ID (B) | |
| AIRE.02 | M | 51 | CMC, HP, AI, TF, Gastritis, EH, HTN, SS, Hpit, | D |
| | | | Pneumonitis, Vitiligo, B12 def, ID (D) | |
| AIRE.03 | F | 19 | HP, AI, POI, TIN, EH, HTN, Pneumonitis, Urticarial | D |
| | | | eruption | |
| AIRE.52 | F | 9 | HP, HT, EH, Urticarial eruption, Vitiligo, ID (D) | D |
| AIRE.53 | F | 8 | CMC, HP, AI, HT, EH, HTN, Urticarial eruption, ID (D) | V |
| AIRE.58 | M | 16 | CMC, HP, AI, TF, Gastritis, EH, ND, KC, Alopecia, B12 | V |
| | | | def, ID (D) | |
| AIRE.59 | M | 7 | CMC, HP, AI, EH, ND, Alopecia, ID (D) | V |
| AIRE.60 | M | 19 | CMC, ND, EH, Alopecia, ID (C) | V |
| AIRE.61 | F | 54 | CMC, HP, AI, EH, SS, Pneumonitis, ID (C) | V |
| AIRE.62 | F | 15 | AI, AIH, HT, Gastritis, Pneumonitis, Urticarial eruption, ID | V |
| | | | (C) | |
| AIRE.55 | M | 19 | CMC, HP, AI, Gastritis, EH, Urticarial eruption, Alopecia | V |
| AIRE.69 | M | 18 | CMC, AI, AIH, ID (B) | V |
| AIRE.56 | M | 2 | AIH, EH, Urticarial eruption, ID (D) | V |
| AIRE.54 | F | 7 | CMC, HP, AI, EH, Pneumonitis, Urticarial eruption | V |
| AIRE.63 | F | 15 | CMC, HP, AI, EH, B12 def, ID (B) | V |
| AIRE.71 | F | 30 | CMC, HP, Gastritis, EH, Pneumonitis, Vitiligo, ID (D) | V |
| AIRE.71B | M | 15 | CMC, AI, HT, Gastritis, ND, Pneumonitis, Alopecia | V |
| AIRE.74 | F | 11 | CMC, HP, AI, HT, Gastritis, TIN, EH, SS, Pneumonitis, | V |
| | | | Urticarial eruption, Alopecia, ID (C) | |
| AIRE.68 | F | 15 | CMC, AI, Gastritis, EH, SS, Pneumonitis, B12 def, ID (C) | V |
| AIRE.70 | F | 16 | CMC, SS, Urticarial eruption, B12 def, ID (C) | V |
| AIRE.66 | M | 13 | CMC, HP, AI, DM, EH, Urticarial eruption, Alopecia | V |
| AIRE.67 | M | 20 | CMC, HP, AI, Pneumonitis, Urticarial eruption, Vitiligo, ID | V |
| | | | (D) | |
| AIRE.87 | F | 15 | CMC, HP, AI, AIH, HT, EH, Pneumonitis, Vitiligo, B12 | V |
| | | | def, ID (B) | |
| AIRE.65C | M | 2 | CMC, HP, AI, Urticarial eruption, ID (C) | V |
| AIRE.65B | M | 6 | CMC, HP, AI, EH | V |
| AIRE.65 | F | 11 | CMC, HP, AI, EH, Urticarial eruption, Vitiligo, GH def, ID | V |
| | | | (D) | |
| AIRE.73 | F | 13 | CMC, HP, AIH, HT, POI, EH, ID (B) | V |
| AIRE.76 | M | 10 | CMC, HP, Urticarial eruption, Vitiligo, ID (D) | V |
| AIRE.86 | F | 3 | HP, Urticarial eruption, ID (C) | V |
| AIRE.77 | M | 10 | HP, AIH, HT, SS, Pneumonitis, Vitiligo, | V |
| | | | Alopecia, ID (D) | |
| AIRE.78 | M | 2 | HP | V |
| AIRE.79 | M | 10 | CMC, HP, AI, AIH, EH, Urticarial eruption, GH def, | V |
| | | | Asplenia | |

ND, nail dystrophy. HP, hypoparathyroidism. KC, keratoconjunctivitis. CMC, chronic mucocutaneous candidiasis. ID (D, C, B), Intestinal dysfunction (diarrheal-type, constipation-type, both). AIH, autoimmune hepatitis. POI, primary ovarian insufficiency. HTN, hypertension. HT, hypothyroidism. B12 def, B12 (vitamin) deficiency. DM, diabetes mellitus. SS, Sjogren's-like syndrome. GH def, Growth hormone deficiency. AI, Adrenal Insufficiency. EH, (dental) enamel hypoplasia. TF, testicular failure. TIN, Tubulointerstitial Nephritis. Hpit, Hypopituitarism.
D, Discovery cohort; V, Validation cohort.
*Age at most recent evaluation prior to Dec. 30, 2019

In this patient cohort, the clinical phenotypes included: nail dystrophy (n=15, 22%), hypoparathyroidism (n=54, 81%), keratoconjunctivitis (n=14, 21%), chronic mucocutaneous candidiasis (n=55, 82%), intestinal dysfunction (diarrheal-type, constipation-type, both) (n(all)=54, 81%), autoimmune hepatitis (n=23, 34%), primary ovarian insufficiency (n=10, 15%), hypertension (n=12, 18%), hypothyroidism (n=16, 24%), vitamin B12 deficiency (n=17, 25%), diabetes mellitus (n=6, 9%), Sjogren's-like syndrome (n=24, 36%), growth hormone deficiency (n=7, 10%), adrenal insufficiency (n=53, 79%), dental enamel hypoplasia (n=53, 79%), testicular failure (n=6, 6%), tubulointerstitial nephritis (n=3, 4%), hypopituitarism (n=1, 1%), vitiligo (n=22, 33%), gastritis (n=25, 37%), urticarial eruption (n=39, 58%), alopecia (n=18, 27%), asplenia (n=5, 7%), and pneumonitis (n=25, 37%).

### Example 3. Detection of Literature-Reported APS1 Autoantigens

PhIP-seq results were first cross referenced with previously reported APS1 autoantibody targets (Alimohammadi et al., 2008, 2009; Clemente et al., 1997; Fishman et al., 2017; Hedstrand et al., 2001; Husebye et al., 1997; Kluger et al., 2015; Kuroda et al., 2005; Landegren et al., 2016, 2015; Leonard et al., 2017; Meager et al., 2006; Meyer et al., 2016; Oftedal et al., 2015; Pontynen et al., 2006; Sansom et al., 2014; Shum et al., 2013, 2009; Soderbergh et al., 2004). A total of 23 known autoantibody specificities were detected by this assay given the criteria described in Examples 1 and 2 above: IL22, IFNA6, IFNA2, IFNA5, IL17A, IFNA13, CYP21A2, CYP17A1, CYP11A1, MAGEB1, NLRP5, KCNRG, PDILT, IL17F, SPAG16, LCN1, DDC, CYP1A2, GGA1, SYNPR, SOX10, ZNF410, SOX9. Importantly, many of the well-validated APS1 antigens, including specific members of the cytochrome P450 family (CYP1A2, CYP21A1, CYP11A1, CYP17A1), lung-disease associated antigen KCRNG, as well as IL17A, IL17F, and IL22, among others were well represented (data not shown). In contrast, the diabetes-associated antigens GAD65 and INS did not meet these stringent detection criteria and only weak signal was detected to many of the known interferon autoantibody targets known to be present in many APS1 patients (data not shown), perhaps due to the conformational nature of these autoantigens (Bjork et al., 1994; Meager et al., 2006; Meyer et al., 2016; Wolff et al., 2013; Ziegler et al., 1996). Other APS1 associated antigens assessed were: LDHC, BPIFB1, TGM4, GAD1, ATP4B, IFNA17, SPANXN4, PGM2, TEX264, APOBEC3A, PAH, TG, GSN, IFNA8, RPL12, MOB4, RFCS, GAGE1, RALA, GABPB2, FGF12, PTPRN, DCTC, MAGEB2, CYP2B6, DSG3, ZAN, TH, INS, FUT9, RPB3, SF3B2, HMGB1, CRYGD, AQP2, MAGEA4, TPH1, CAPNS1, SPANXD, TPO, MBIP, GNG4, IFNW1, MAGEA10, MAGEA3, UBL3, SP1, CYP2A6, PVALB, DDX43, GIF, BPIFA2, HSD3B2, CASR, SPAG8, GAGE12G, LDLRAD4, VWC2, ATP4A, TSGA10, MAPK14, HYPK, PMP2, ICA1, MED1, C17orf49, RSU1, TSPY3, DGCR8, GSTM4, ABHD11, C9orf142, CXXC4, IFNA14, GSTO1, IFNA4, GAD2, SPTAN1, MKNK2, IFNA21, POMZP3 (data not shown).

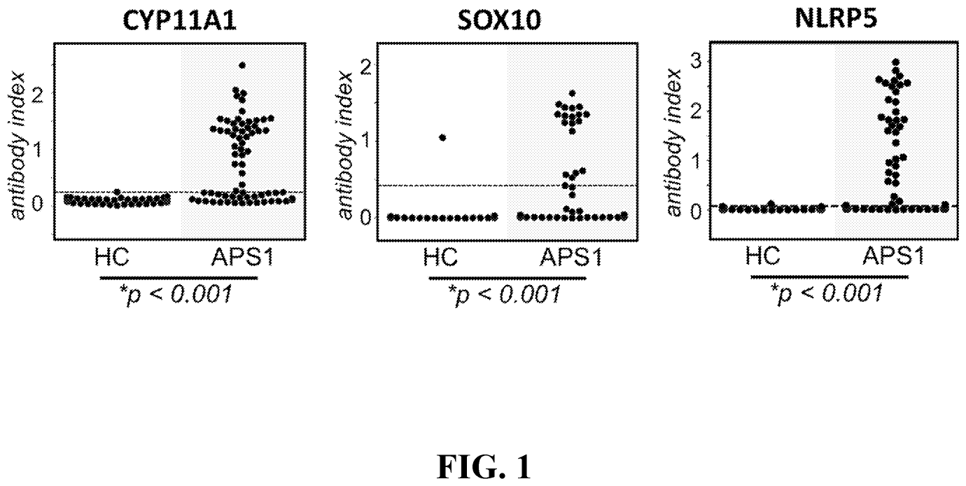

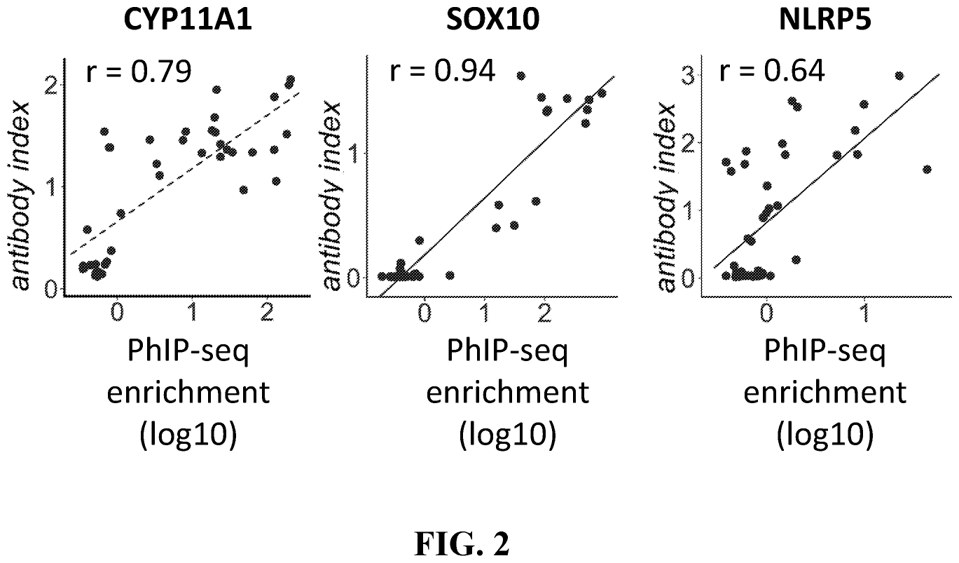

Three known autoantigens that were prevalent within the patient cohort were selected in order to determine how PhIP-seq performed against an orthogonal whole protein-based antibody detection assay. A radioligand binding assay (RLBA) was performed by immunoprecipitating in vitro transcribed and translated S35-labeled proteins CYP11A1, SOX10, and NLRP5 with APS1 serum (Alimohammadi et al., 2008; Berson, Yalow, Bauman, Rothschild, & Newerly, 1956; Hedstrand et al., 2001; Winqvist, Gustafsson, Rorsman, Karlsson, & Kampe, 1993). Importantly, and in contrast to PhIP-seq, this assay tests for antibody binding to full-length protein. As shown in FIG. 1, by RLBA, these three antigens were present in and specific to both the initial discovery APS1 cohort (n=39) as well as the expanded validation cohort (n=28), but not healthy control sera (n=61). Furthermore, direct comparison of the PhIP-seq data to RLBA revealed an excellent correlation across individual APS1 patient sera, as shown in FIG. 2. Individual patient PhIP-seq enrichments values (log 10) were plotted as compared to radiolignd binding assay antibody index values for the antigens. The Pearson correlation coefficient r was determined to be 0.94 for SOX10 and 0.64 for NLRP5 (data not shown). Together, these results demonstrate that PhIP-seq detects known autoantigens and that PhIP-seq results validate well in orthogonal whole protein-based assays.

To determine whether the PhIP-seq APS1 dataset could yield higher resolution information on antigenic peptide sequences with respect to previously reported targets, the normalized enrichments of all peptides belonging to known disease-associated antigens CYP11A1 and SOX10 were mapped across the full length of their respective proteins. As described in Example 1, PhIP-seq enables 49 amino acid resolution of antibody signal from APS1 sera to each peptide as compared to signal from mock-IP (log 10 scale). The antigenic regions within these proteins were observed to be similar across all patients positive for anti-CYP11A1 and anti-SOX10 antibodies, respectively (data not shown) suggesting peptide-level commonalities and convergence among the autoreactive antibody repertoires across patients. These data suggest that patient autoantibodies to these proteins often target similar, but not identical protein regions. Of interest, the antigenic region of CYP11A1 was found to be primarily the 100 amino acid residues at the N′ terminus, while the there were multiple antigenic regions identified for the SOX10 protein (data not shown).

### Example 4. Identification of Novel APS1 Autoantigens

Having confirmed that PhIP-seq analysis of APS1 sera detected known antigens, the same data were then investigated for the presence of novel, previously uncharacterized APS1 autoantigens. To avoid false positives, a more conservative set of criteria than used in Examples 1-3 were used as follows: a minimum of 3/39 APS1 patients and 0/28 non-APS1 controls had to exhibit normalized gene counts in the immunoprecipitation with greater than 10-fold enrichment as compared to the control set of 17 mock-IP (beads, no sera) samples; statistical analysis: hierarchically clustered (Pearson) Z-scored. This simple, yet stringent criteria yielded 81 genes, which included 12 known antigens (MAGEB10, MAGEB17, MAGEB1, MAGEB4, IL22, IFNA5, DDC, SOX10, CYP11A1, LCN1, IL17F, CYP17A1) and 69 novel antigens (BNIP1, RASIP1, RNF165, TBATA, TCOF1, KHDC3L, PNO1, CDK5R1, FAM47A, SPEF2, SNRPD1, RBXML3, RBXML2, PDYN, CYSRT1, SARNP, PRR11, AP5B1, C19orf80, NOP2, CRH, NOG, SAMD4A, SRRT, CATSPER2, DAZ4, DAZ2, DAZ1, ARFRP1, C7orf50, C3orf30, RFX4, CAMK2N1, ZNF367, ZNF439, POU1F1, HAPLN1, MUC5B, RFX6, SRSF8, ARRDC3, ACPT, PLAGL2, CDK2, NOP16, DEDD2, SUGP2, STK19, SAYSD1, BCL9L, GET4, FAIM3, ASMT, MORC2, TRIM74, TRIM50, POLDIP3, CROCC2, NBPF19, SOX8, SLC18A1, GIP, NKX6-3, QSER1, H1F0, ZNF618, AOAH, SLX4IP, NANOG) (data not shown).

The most commonly held hypotheses regarding the nature and identity of proteins targeted by the aberrant immune response in APS1 are that targeted proteins (1) tend to exhibit Aire-dependent thymic expression and (2) have restricted expression to one or few peripheral organs and tend not to be widely or ubiquitously expressed. In order to systematically address the question of whether the identified novel antigens are also preferentially tissue-restricted, tissue-specific RNA expression was assessed using a consensus expression dataset across 74 cell types and tissues (Uhlen et al., 2015). For each gene, the ratio of expression in the highest tissue as compared to the sum of expression across all tissues was calculated, resulting in higher ratios for those mRNAs with greater degrees of tissue-restriction. Using this approach, the mean tissue-specificity ratio of the 82 PhIP-seq positive antigens was increased by approximately 1.5-fold (p=0.0017) as compared to the means from iterative sampling (n=10,000 samplings) of 82 genes (data not shown).

### Example 5. Identification of Novel Antigens Common to Many Patients

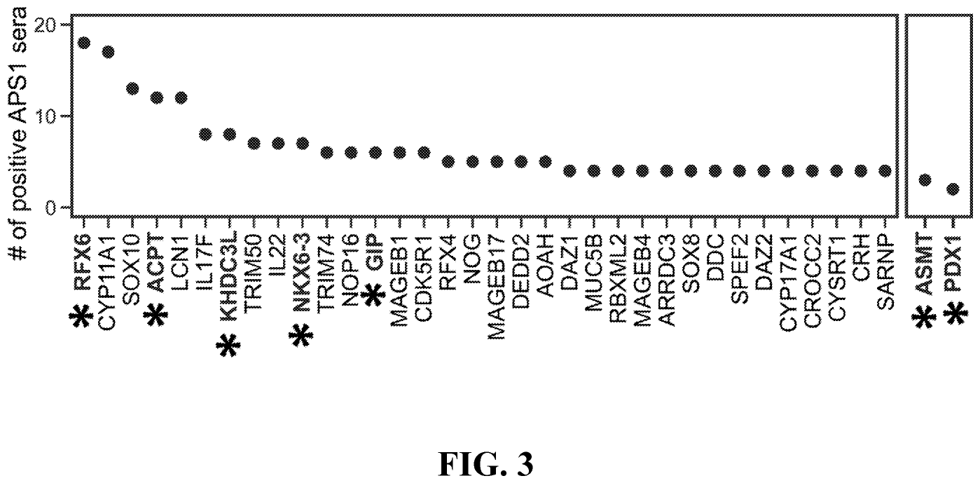

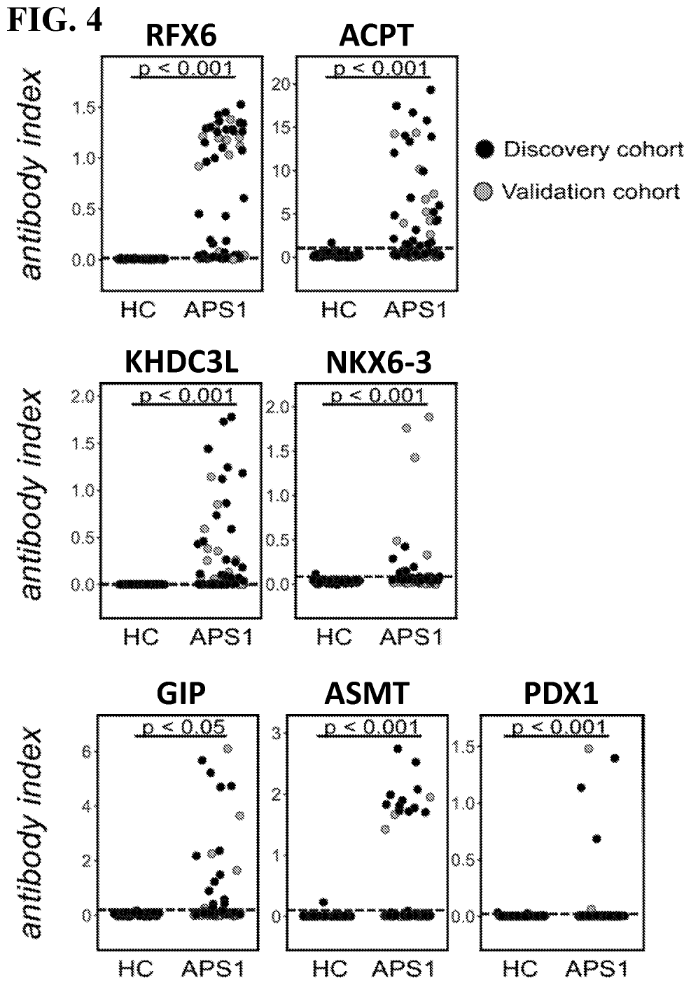

Identified antigens were ranked by frequency within the cohort. As seen in FIG. 3, five antigens were positive in ten or more patients, including two novel antigens. In addition, the majority of antigens found in 4 or more APS1 patient sera were novel. Five of the most frequent novel antigens were selected for subsequent validation and follow-up. These are marked with asterisks (*) in FIG. 3 and included RFX6, a gene implicated in pancreatic and intestinal pathology (Patel et al., 2017; S. B. Smith et al., 2010); ACPT, a gene implicated in dental enamel hypoplasia (Choi, Kim, Yun, Kim, & Cho, 2016; Seymen et al., 2016; C. E. Smith et al., 2017); KHDC3L, a gene with oocyte-restricted expression (Li, Baibakov, & Dean, 2008; Zhang et al., 2018; Zhu et al., 2015); NKX6-3, a gastrointestinal transcription factor (Alanentalo et al., 2006); and GIP, a peptide involved in intestinal motility and energy homeostasis (Adriaenssens et al., 2019; Moody, Thim, & Valverde, 1984; Pederson & McIntosh, 2016). Several less frequent (but still shared) novel antigens were also chosen for validation. These antigens are marked with asterisks (*) in FIG. 3 and included ASMT, a pineal gland gene involved in melatonin synthesis (Ackermann et al., 2006; Rath et al., 2016); and PDX1, a master regulator in the pancreas (Holland, Hale, Kagami, Hammer, & MacDonald, 2002; Stoffers, Zinkin, Stanojevic, Clarke, & Habener, 1997). Using a whole-protein radiolabeled binding assay (RLBA) for validation, all seven proteins were immunoprecipitated by antibodies in both the PhIP-seq APS1 discovery cohort (n=39), as well as in the validation cohort of APS1 patient sera that had not been interrogated by PhIP-seq (n=28). As shown in FIG. 4, whereas an expanded set of healthy control sera (n=61) produced little to no immunoprecipitation signal by RLBA as compared to positive control antibodies (low antibody index), APS1 sera yielded significant immunoprecipitation signal enrichment for each whole protein assay (high antibody index).

The PhIP-seq data were compared to results from the RLBA (n=39, discovery cohort only) and yielded positive correlations between the two datasets (RFX6: r=0.95, ACPT: r=0.92, KHDC3L: r=0.58, NKX6-3: r=0.61, GIP: r=0.90, ASMT: r=0.61, PDX1: r=0.90; data not shown). Notably, for some genes, such as NLRP5 (shown in FIG. 2), and particularly for ASMT (shown in FIG. 3 and FIG. 4), the RLBA results revealed additional autoantibody-positive samples not detected by PhIP-seq. Of note, this group of seven novel antigens all exhibited either tissue enriched, tissue enhanced, or group enhanced expression according to the Human Protein Atlas database, whereas only 34% of all genes would normally fall within these categories (Uhlen et al., 2015) (see Table 5 below).

TABLE 5
Tissue-restricted expression patterns
of validated novel APS1 antigens.

| | Protein Atlas: | |
|---|---|---|
| | RNA specificity | |
| Gene | category | |
| (Human/mouse) | (Tissue)¹⁴ | Selected literature annotations |
| RFX6/Rfx6 | Tissue enhanced | Pancreas - Islets (Piccand et al., |
| | | 2014; S. B. Smith et al.,2010) |
| | | Intestine - Enteroendocrine cells |
| | | (Gehart et al., 2019; Piccand et |
| | | al., 2019; S. B. Smith et al., |
| | | 2010) |
| KHDC3L/Khdc3 | Group enriched | Ovary - Oocytes (Y. Zhang et al., |
| | | 2018; K. Zhu et al., 2015) |
| ACPT/Acpt | Tissue enhanced | Testes (Yousef, Diamandis, Jung, |
| | | & Diamandis, 2001) Dental enamel |
| | | (Green et al., 2019; Seymen et |
| | | al., 2016; C. E. Smith et al., |
| | | 2017) |
| ASMT/Asmt | Tissue enhanced | Brain - Pineal Gland (Rath et al., |
| | | 2016) |
| GIP/Gip | Tissue enriched | Intestine - Enteroendocrine cells |
| | | (Moody, Thim, & Valverde, 1984) |
| NKX6-3/Nkx6-3 | Group enriched | Pancreas - PP-cells (Schaum et al., |
| | | 2018) Intestine (Alanentalo et al., |
| | | 2006) |
| PDX1/Pdx1 | Group enriched | Pancreas - Islets (Holland et al., |
| | | 2002; Staffers et al., 1997) |

### Example 6. Autoantibody-Disease Associations for Both Known and Novel Antigens

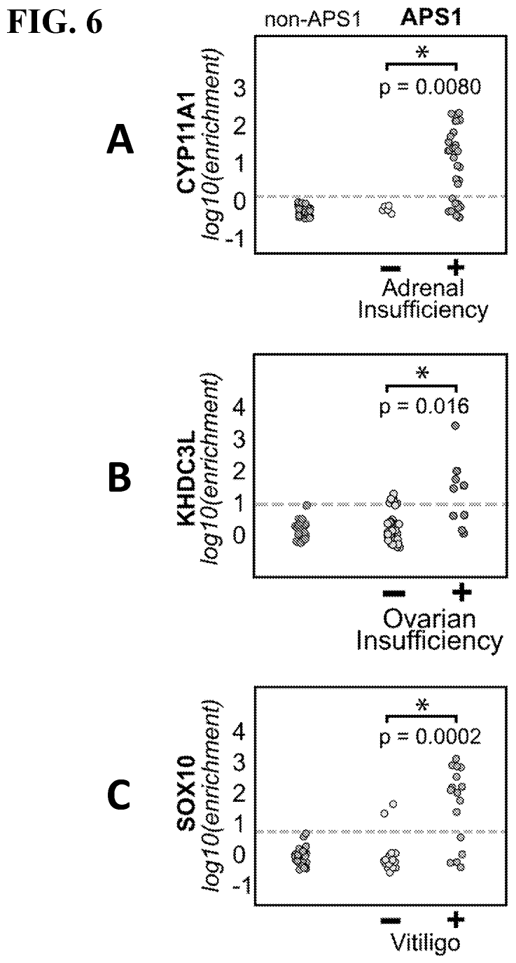

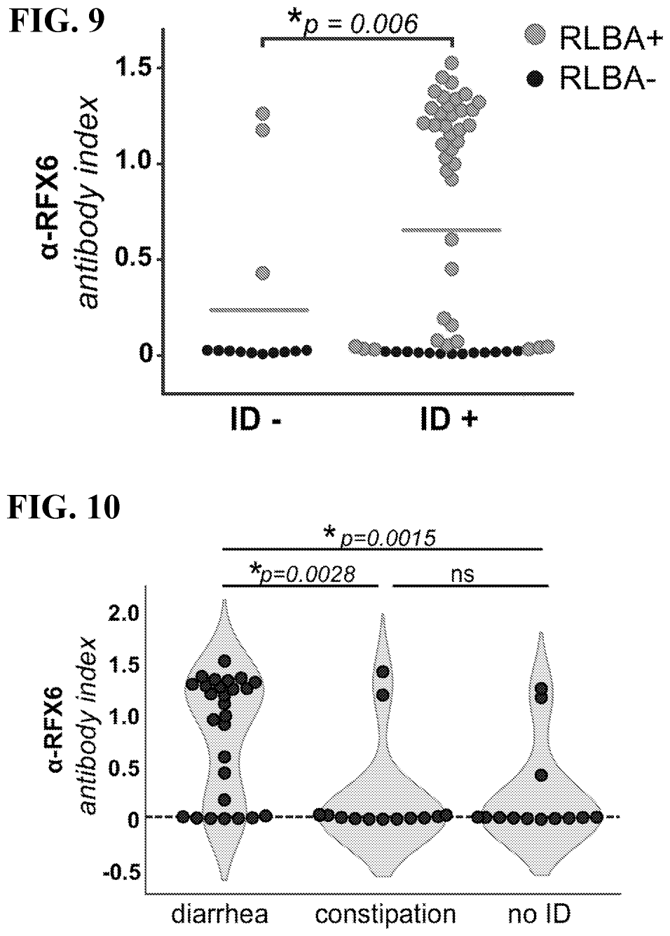

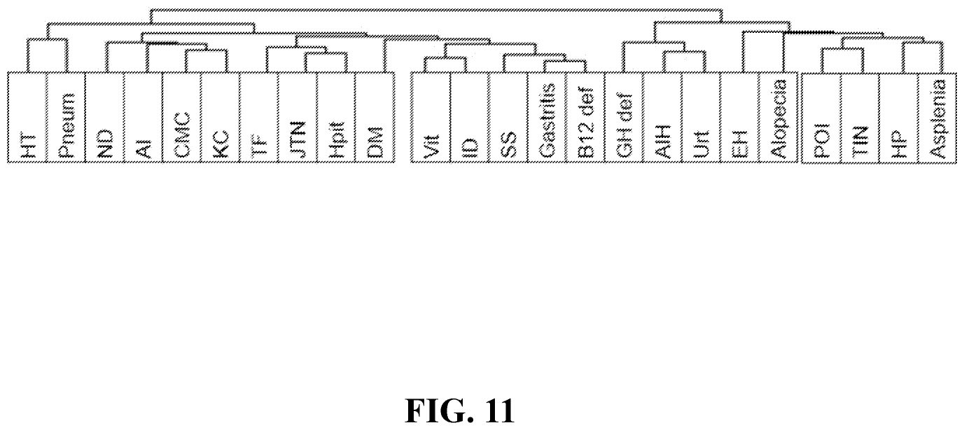

Because the patients in this APS1 cohort have been extensively phenotyped for 24 clinical manifestations, the PhIP-seq APS1 data were queried for associations with the following phenotypic conditions: nail dystrophy, hypoparathyroidism, keratoconjunctivitis, chronic mucocutaneous candidiasis, intestinal dysfunction (diarrheal-type, constipation-type, both), autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstitial nephritis, hypopituitarism, vitiligo, gastritis, urticarial eruption, alopecia, asplenia, and pneumonitis. Several autoantibody specificities, both known and novel, were found to possess highly significant associations with several clinical phenotypes, as shown in FIG. 5. Clustered disease correlations in the patient cohort were also assessed as shown in FIG. 11 (n=67; Spearman's rank correlation not shown). Among these associations, three are described in more detail and illustrated in FIG. 6: CYP11A1 (also known as cholesterol side chain cleavage enzyme) with adrenal insufficiency (AI), KHDC3L with ovarian insufficiency, and SOX10 with vitiligo (marked as “A”, “B” and “C”, respectively, in FIG. 5 and FIG. 6). Additionally, as discussed in Example 10 below, RFX6 is associated with diarrheal-type intestinal dysfunction (marked with arrowhead in FIG. 5 and detailed in FIG. 9 and FIG. 10). Strikingly, anti-CYP11A1 antibodies are present in AI and are known to predict disease development (Betterle et al., 2002; Obermayer-Straub et al., 2000; Winqvist et al., 1993). Similarly, antibodies to SOX10, a master regulator of melanocyte fate, have been previously shown to correlate with the presence of autoimmune vitiligo (Hedstrand et al., 2001).

### Example 7. Anti-KHDC3L Antibodies in APS1-Associated Ovarian Insufficiency

Primary ovarian insufficiency is a highly penetrant phenotype, with an estimated 60% of female APS1 patients progressing to an early, menopause-like state (Ahonen et al., 1990; Ferré et al., 2016). Interestingly, a set of 5 genes (KHDC3L, SRSF8, PNO1, RASIP1, and MORC2) exhibited a significant association with ovarian insufficiency in this cohort, as seen in FIG. 5. A publicly available RNA-sequencing dataset from human oocytes and supporting granulosa cells of the ovary confirmed that of these 5 genes, only KHDC3L exhibited expression levels in female oocytes comparable to the expression levels seen for the known oocyte markers NLRP5 and DDX4 (Zhang et al., 2018). Specifically, KHDC3L, NLRP5, and DDX4 are highly expressed in oocytes relative to SRSF8, PNO1, RASIP1, MORC2, and known granulosa cell markers INHBB and AMH. In granulosa cells, SRSF8, PNO1, INHBB, and AMH are highly expressed relative to KHDC3L, RASIP1, MORC2, NLRP5, and DDX4 (data not shown). Therefore, the relationship between anti-KHDC3L antibodies and ovarian insufficiency in the patient cohort was further investigated.

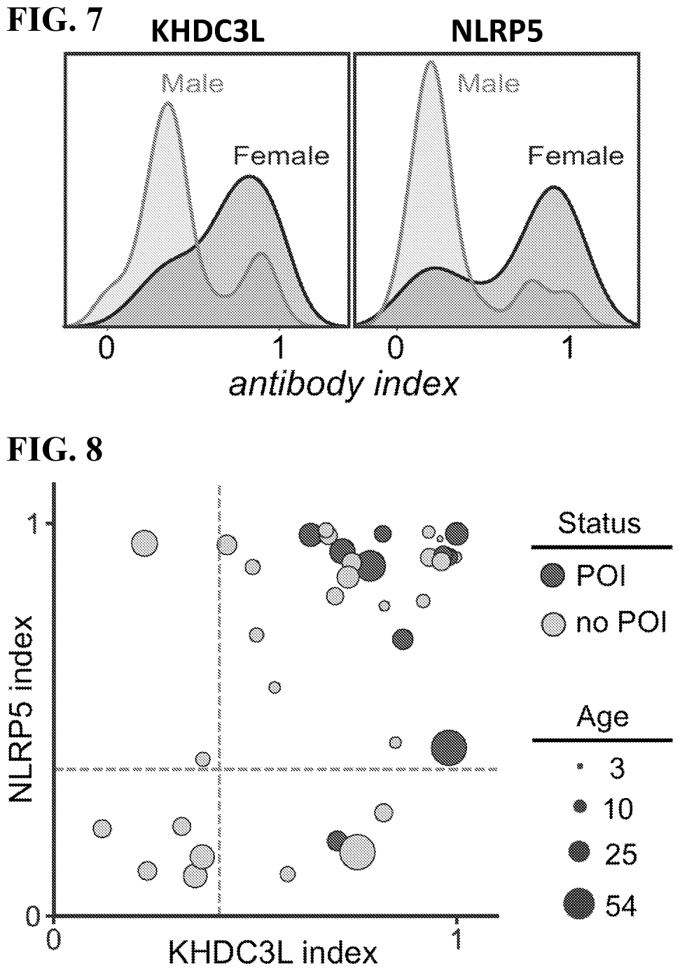

KHDC3L is a well-studied molecular binding partner of NLRP5 within the ovary (Li et al., 2008; Zhu et al., 2015). Together, NLRP5 and KHDC3L form part of a critical oocyte-specific molecular complex, termed the subcortical maternal complex (SCMC) (Bebbere et al., 2016; Brozzetti et al., 2015; Li et al., 2008; Liu et al., 2016; Zhu et al., 2015). Furthermore, knockout of NLRP5 and KHDC3L in female mice results in fertility defects, and human genetic mutations in these genes of the SCMC have been linked to infertility and molar pregnancies (Akoury et al., 2015; Li et al., 2008; Reddy et al., 2012; Wang et al., 2018; W. Zhang et al., 2019). Interestingly, previous work established NLRP5 as a parathyroid-specific antigen in APS1, with potential for additional correlation with ovarian insufficiency (Alimohammadi et al., 2008). However, anti-NLRP5 antibodies lack sensitivity for ovarian insufficiency. Importantly, unlike NLRP5, KHDC3L is expressed primarily in the ovary, and thus represents a potential oocyte-specific autoantigen (Liu et al., 2016; Virant-Klun et al., 2016; Y. Zhang et al., 2018). A publicly available RNA-sequencing dataset from human oocytes and supporting granulosa cells of the ovary confirmed that KHDC3L, as well as NLRP5 and the known oocyte marker DDX4, are highly expressed within the oocyte population, but not in the supporting granulosa cell types (Y. Zhang et al., 2018) (data not shown). Interestingly, the majority (64%) of patient sera had a concordant status for antibodies to KHDC3L and NLRP5 (data not shown). Although previous reports did not find a strong gender prevalence within patients positive for anti-NLRP5 antibodies, the mean anti-NLRP5 and anti-KHDC3L antibody signals were increased in female patients in this cohort (by 1.6- and 2.1-fold, respectively), as shown in FIG. 7. Finally, all 10 female patients in the expanded cohort with diagnosed ovarian insufficiency were also positive for anti-KHDC3L antibodies, resulting in a remarkable (100%) sensitivity for ovarian insufficiency, as shown in FIG. 8.

### Example 8. High Prevalence of Anti-ACPT Antibodies

Similar to known antigens CYP11A1, SOX10, LCN1, the novel antigen ACPT was found to occur at high frequencies in this cohort, as shown in FIG. 3. ACPT is highly expressed in dental enamel, and familial mutations in ACPT result in dental enamel hypoplasia similar to the enamel hypoplasia seen in ˜90% of this APS1 cohort (Seymen et al., 2016; C. E. Smith et al., 2017). Strikingly, 50% of patients were positive for anti-ACPT antibodies by RLBA (see FIG. 4), with excellent correlation between RLBA and PhIP-seq data (r=0.92; data not shown). Consistently, APS1 patients with enamel hypoplasia exhibited a trend towards higher anti-ACPT antibody signal by RLBA (p=0.064; data not shown).

### Example 9. High Prevalence of Anti-RFX6 Antibodies

In this cohort, 82% (55/67) of APS1 sera exhibited signal that was at least 3 standard deviations above the mean of non-APS1 control signal due to the extremely low RFX6 signal across all healthy controls by RLBA, as shown in FIG. 4. Using a more stringent cutoff for RFX6 positivity by RLBA at 6 standard deviations above the mean, 65% of patients were positive for anti-RFX6 antibodies. RFX6 is expressed in both intestine and pancreas, and loss of function RFX6 mutations in humans lead to both intestinal and pancreatic pathology (Gehart et al., 2019; Patel et al., 2017; Piccand et al., 2019; S. B. Smith et al., 2010). Interestingly, across all patients with anti-RFX6 antibodies, the response targeted multiple sites within the protein (at the N′ terminal region within the first 150 amino acid residues and across the C′ terminal third of the protein), suggesting a polyclonal antibody response (data not shown).

### Example 10. Anti-Enteroendocrine and Anti-RFX6 Response in APS1

The extent and frequency of intestinal dysfunction in APS1 patients has only recently been clinically uncovered and reported, and therefore still lacks unifying diagnostic markers as well as specific intestinal target antigen identities (Ferré et al., 2016). This investigation of APS1 sera revealed several antigens that are expressed in the intestine, including RFX6 and GIP, and the question of whether or not autoimmune response to RFX6+ cells in the intestine was involved in APS1-associated intestinal dysfunction was investigated. Using a publicly available murine single-cell RNA sequencing dataset of 16 different organs and over 120 different cell types, RFX6 expression was confirmed to be present in and restricted to pancreatic islets and intestinal enteroendocrine cells (Schaum et al., 2018) (single cell RNA expression data not shown). Serum from a patient with APS1- associated intestinal dysfunction and anti-RFX6 antibodies was next tested for reactivity against human intestinal enteroendocrine cells, revealing strong nuclear staining that consistently colocalized with ChromograninA (ChgA), a well-characterized marker of intestinal enteroendocrine cells (Goldspink et al., 2018; O'Connor et al., 1983) (single cell RNA expression data not shown). In contrast, little to no intestinal staining was observed from APS1 serum that lacked anti-RFX6 antibodies and from non-APS1 patient sera. (single cell RNA expression data not shown). Furthermore, serum from patients with anti-RFX6 antibodies stained tissue culture cells transfected with FLAG-tagged RFX6, and serum from APS1 patients without anti-RFX6 antibodies did not stain tissue culture cells transfected with FLAG-tagged RFX6 (data not shown). Immunofluorescence conditions and secondary antibodies were confirmed to show no background signal or cross-reactivity by lack of signal in control reactions testing the following conditions: 1) no primary antibody and no secondary antibody; 2) no primary antibody and both secondary antibodies; 3) human serum primary antibody and anti-rabbit secondary antibody; and 4) rabbit anti-Flag primary antibody and anti-human secondary antibody (data not shown). These results support the notion that there exists a specific antibody signature, typified by anti-RFX6, associated with enteroendocrine cells in APS1 patients.

Mice with induced deletion of Rfx6 feature malabsorption and soft stool, and humans with enteroendocrine cell deficiency also feature chronic diarrhea (Hogenauer et al., 2001; Oliva-Hemker et al., 2006; Piccand et al., 2019; Posovszky et al., 2012; S. B. Smith et al., 2010; J. Wang et al., 2006). In this cohort, 54/67 (81%) of patients have intestinal dysfunction defined as the presence of chronic diarrhea, chronic constipation or an alternating pattern of both, without meeting ROME III diagnostic criteria for irritable bowel syndrome, as previously described (Ferré et al., 2016). When patients were subsetted by presence or absence of intestinal dysfunction, the anti-RFX6 RLBA signal as measured by radioligand binding assay was significantly higher for patients with intestinal dysfunction, as shown in FIG. 9. Further subsetting of patients by subtype of intestinal dysfunction, as shown in FIG. 10, revealed that patients positive for anti-RFX6 antibodies belonged preferentially to the diarrheal-type (as opposed to constipation-type) group of intestinal dysfunction. In contrast, antibodies to the known intestinal antigen TPH1 were distributed less specifically across both types (diarrheal- and constipation-type) of ID, despite a high frequency of both anti-TPH1 and anti-RFX6 in the cohort (55/67 for anti-RFX6, 45/67 for anti-TPH1). These observations are consistent with a previous report that anti-TPH1 antibodies show an association with ID, but not specifically with the diarrheal subtype (Kluger et al., 2015). Given that RFX6 is also expressed in the pancreas, the association of anti-RFX6 antibodies with type 1 diabetes was also examined. Six out of seven APS1 patients with type 1 diabetes (6 diagnosed prior to serum draw, 1 diagnosed post serum draw) exhibited a high level of anti-RFX6 antibodies as measured by radioligand binding assay (data not shown). Together, these data suggest that RFX6 is a common, shared autoantigen in APS1 that may be involved in the immune response to intestinal enteroendocrine cells as well as pancreatic islets. Future studies will help to determine whether testing for anti-RFX6 antibodies possesses clinical utility for prediction or diagnosis of specific APS1 autoimmune disease manifestations as well as for non-APS1 isolated autoimmune disease.

### Example 11. Identification of PLIN1 as an Autoantigen in APS1: Approach and Methods

Examples 11-14 describe the discovery of autoantibodies against PLIN1 in a genetic mouse model of autoimmunity, and the presence of antibodies to PLIN1 in an APS1 patient.

Phage-Display and Immunoprecipitation protocol (PhIP-Seq): The T7 Phage Display library (PhIP-Seq library) used in this study was validated previously (O'Donovan et al., 2018) and can be accessed at www.github.com/derisilab-ucsf/PhIP-PND-2018. The experiment is performed as detailed in Mandel-Brehm et al. (2019) and Vazquez et al. (2020). See also WO 2020/190700, which is incorporated by reference in its entirety herein. However, instead of one microliter of human sera per immunoprecipitation (IP), we use one microliter of sera from mice. Four AIRE knockouts and three age-matched wildtype controls were tested. Briefly, mouse sera were mixed with one milliliter of PhIP-Seq library(l000 pfu/mL) and incubated for 12-18 hours at 4° C. Antibody-bound phage were immunoprecipitated using a mix of protein A and protein G magnetic beads (Thermo Fisher), eluted and DNA sequenced to identify the unknown phage antigen(s). First, DNA sequencing reads were converted to “peptide reads” by aligning to the reference library and normalized total reads to 100,000 reads (RP100K) for each sample. Then the total number of peptide reads was summed with respect to annotated proteins (˜20,000 unique proteins represented in the library). The criteria for candidate antigens were an RP100K greater than 50, present in greater than two AIRE knockout (“KO”) mice, and not present in any wildtype mice.

Validation of PLIN1 antibodies using 293T expression system: Full-length human and mouse PLIN1 ORF clone was purchased (Human: Origene Cat. No. RC206292; Mouse: OriGene Cat. No. MR208288). For 293 cell assays with mice and RLBA with humans, the human PLIN1 plasmid was used. For RLBA with mice, the mouse PLIN1 plasmid was used. For the human plasmid, PLIN1 transcription is driven from a CMV6 promoter and the expressed protein is Myc-Flag tagged. The validation protocol including 293T cell transfections with plasmid, whole cell lysate preparation, immunoprecipitations with sera and/or commercial antibodies and western blotting as previously described. See e.g., Mandel-Brehm et al, 2019 and WO 2020/190700, both of which are incorporated by reference in their entireties herein. Whole cell lysates containing overexpressed PLIN1 were made to 1 mg/ml, and 10 microliters was set aside prior to IP for input for western blotting. The seven original archived samples (WT n=3, AIRE KO n=4) and an additional AG bead only control (no mouse sera added to IP) were tested for the ability to IP PLIN1 from the whole cell lysate. One microliter of mouse sera or nothing was added to 500 microliters of whole cell lysate, incubated for 12 hour at 4° C. Antibodies were immunoprecipitated with protein A and protein G beads (as done in PhIP-seq), washed three times with RIPA buffer (140 mM NaCl, 10 mM Tris-HCL, 1.0% Triton-X, 0.1% SDS), and boiled in 2× Laemmli buffer. Entire IP elutions were electrophoresed on a 4-12% Bis-Tris protein gel (NuPAGE), transferred to 0.45 micron nitrocellulose, and immunoblotted with primary anti-Flag antibody (Rabbit anti-Flag, 1:5000, Cell Signaling Technology) and infrared secondary antibody Goat anti-Rabbit IgG-IR800 (LICOR).

Indirect immunofluorescence on mouse enteric tissue: Mice were perfused with 4% Paraformaldehyde and stomach was post fixed for 1 hour, following sucrose/OCT embedding for cryosectioning. Sections were cut 12 μM thick. Indirect immunofluorescent stains were obtained by incubating serum from anti-PLIN1 autoantibody positive sample or control sample or commercial antibody to PLIN1 (Sigma) at a dilution of 1:1000 on tissue section. Samples were washed and then developed with a FITC-conjugated secondary anti-human IgG antibody (Abcam). Images were captured using a Nikon Ti confocal microscope at the UCSF imaging core. Colocalization was assessed qualitatively through visual inspection by experimenter blind to study.

Radioligand binding assay: An expression plasmid containing full-length human PLIN1 coding sequence under the control of a T7 promoter (Origene, RC206292) was sequence verified and used as a DNA template for in vitro translation of the PLIN1 protein. PLIN1 was synthesized in the presence of ³⁵S-Methionine to radiolabel the protein as previously described (Berson et al. 1956, Shum et al. 2013, Vazquez et al., 2020). Individual serum samples from a cohort of normal control patients, patients with identified APS1, all without evidence of lipodystrophy, were incubated with radiolabeled PLIN1 at 4° C. overnight. Antibodies were then immunoprecipitated with protein A/G beads, washed, and total radioactive counts were obtained by scintillation. A known PLIN1-specific antibody (Sigma-Aldrich, # HPA024299) was used as a positive control. The Antibody index was calculated as follows: (sample value−mean blank value)/(positive control antibody value−mean blank value). A cut-off for a positive result was set as 3 standard deviations above the mean for normal controls (indicated by dotted line).

### Example 12. Discovery of Autoantibodies to PLIN1 in Mice and Testing of PLIN1 in APS1

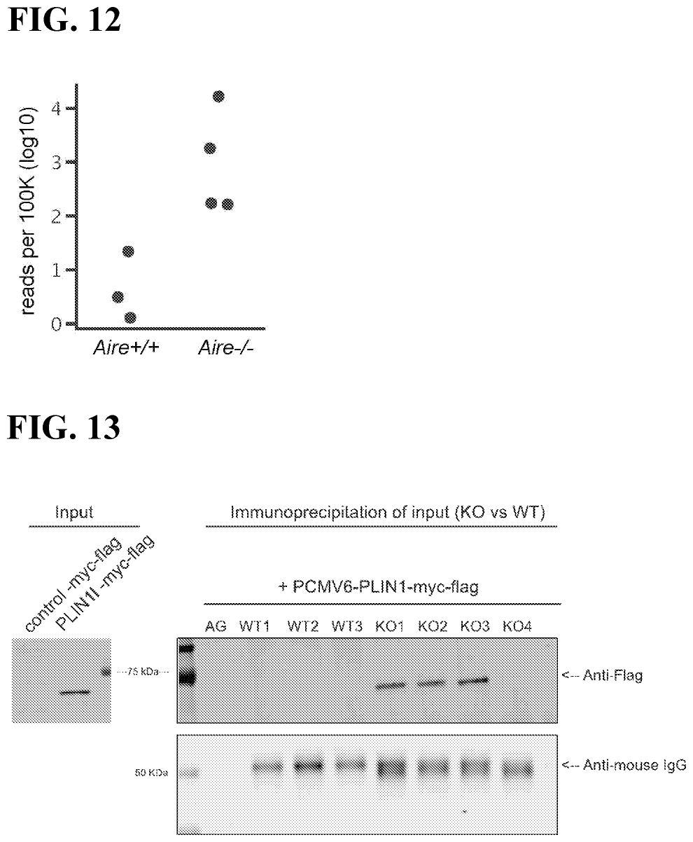

Autoantibodies to PLIN1 were identified by screening sera from murine knockouts of the Aire gene (Aire −/−). Idiopathic antigens in previously banked Aire −/− sera were investigated using PhIP-Seq, a proteome-wide antigen discovery method (Larman et al 2011, see methods). Four Aire −/− as well as three age-matched wildtype controls (Aire +/+) were tested. Datasets were normalized to 100,000 reads and a mean number of reads per protein was calculated for each cohort (see methods). Aire −/− mean RP100Ks were plotted against Healthy mean RP100Ks for each protein in the library (n=100). A candidate antigen was any protein with a positive cutoff of 150 RP100K, that was present in two or more Aire −/− mice and not present in wild-type controls (Aire +/+). Although each sample exhibited distinct patterns of PhIP-Seq reactivity, only one protein, PLIN1, satisfied this stringent criteria (FIG. 12). Peptides contributing to the PhIP-Seq signal for PLIN1 in the four AIRE knockouts are shown in Table 6. Antibody reactivity to full-length PLIN1 in three of four Aire −/− (100% concordance with PhIP-Seq) was validated using 293Tcell overexpression assay and IP elutions from whole cell lysates (See methods, FIG. 13). Because PLIN1 demonstrates subcutaneous fat-specific expression PLIN1-reactive mouse serum was also tested on sections of fat. It was confirmed that sera from Aire −/− mice are reactive to tissue containing fat and further that mouse antibody reactivity co-localizes with reactivity of commercial antibody to PLIN1.

TABLE 6
Peptides contributing to the PhIP-Seq
signal for PLIN1.

| | | SEQ ID |
|---|---|---|
| Fragment | Amino acid sequence | NO |
| PLIN1 | LQLPVVSGTCECFQKTYTSTKEAHPL | 42 |
| Fragment 1 | VASVCNAYEKGVQSASSLAAWSM | |
| PLIN1 | PLVASVCNAYEKGVQSASSLAAWSME | 43 |
| Fragment 2 | PVVRRLSTQFTAANELACRGLDH | |
| PLIN1 | ASVAMQAVSRRRSEVRVPWLHSLAAA | 44 |
| Fragment 11 | QEEGDHEDQTDTEEDTEEEEELE | |
| PLIN1 | HLEEKIPALQYPPEKIASELKDTIST | 45 |
| Fragment 4 | RLRSARNSISVPIASTSDKVLGA | |
| PLIN1 | STRLRSARNSISVPIASTSDKVLGAA | 46 |
| Fragment 5 | LAGCELAWGVARDTAEFAANTRA | |

### Example 13. PLIN1 Expression in Mouse and Human Thymus

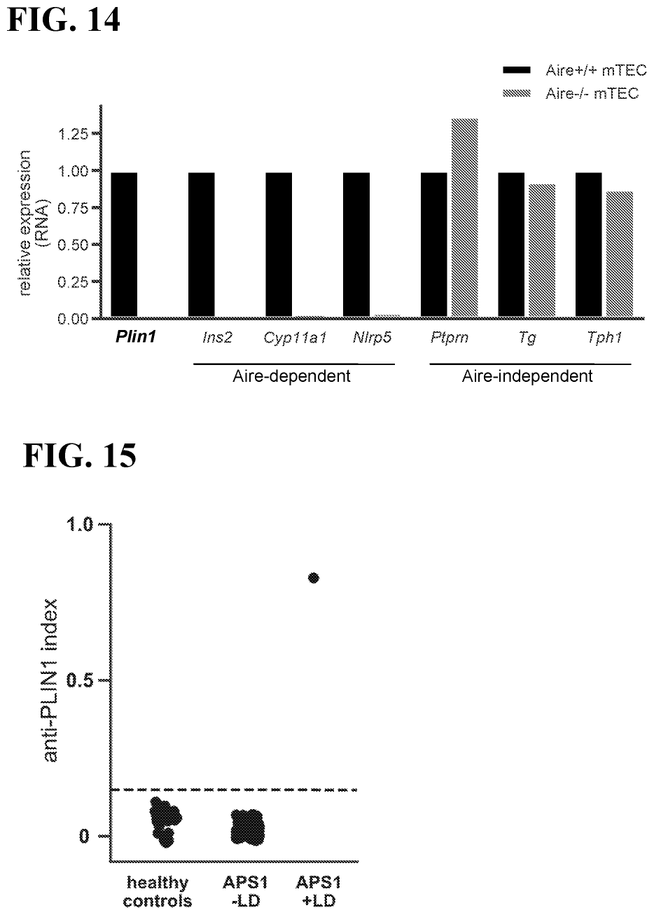

To evaluate whether PLIN1 exhibits Aire-dependent thymic expression, a publicly available bulk RNA-seq dataset previously published by Sansom et al. 2014) was assessed. The relative expression of PLIN1 in medullary thymic epithelial cells (mTECs) isolated from Aire−/− was compared to Aire+/+ mice. As shown in FIG. 14, PLIN1 expression was substantially reduced in the Aire−/− mTECs, similar to the expression patterns exhibited by known Aire-dependent genes Ins2, Cyp11a1, and Nlrp5. These data motivated a furthered candidate exploration of PLIN1 antibodies in human lipodystrophies. A well-established Radioactive Ligand Binding Assay was adapted to screen for antibodies to PLIN1 in humans and set out to test humans with clinically confirmed AGL.

### Example 14. Case Report 1: Genetic APS1 Patient

Clinical phenotyping of APS1 patient—Case report 1. Patient 1, who has been previously described (Sorkina et al. 2016), presented at age 15 months with progressive loss of subcutaneous fat and progressive weight loss. At the age of 4 years, autoimmune hepatitis and the presence or oral candidiasis was noted along with a high fasting insulin level of 40 mIU/1. The patient was started on prednisolone for treatment of the autoimmune hepatitis with improvement of liver function tests. A year later, there was continued loss of subcutaneous fat noted and notable laboratory work included a leptin <1 ng/ml (normal 3.6-11.1) and continued hyperinsulinemia. Given the continued clinical picture of lipodystrophy a genetic panel of candidate genes was tested and found to be negative for any coding mutations (ZMPSTE24, LMNA, BSCL2, PLIN1, PTRF, LMNB2, POLD1, AKT2, CIDEC, PIK3CA, PPARG, PSMB8, CAV1, PPP1R3A, AGPAT2). Shortly after this, the patient developed fatigue, nausea, weight loss and hypotension. Adrenal insufficiency was diagnosed with an elevated ACTH level and treatment with adrenal hormone replacement was initiated. The patient was then tested for a mutation in the AIRE gene and confirmed to harbor a homozygous mutation at c.769C>T p.R257X. Serum from the patient was obtained for autoantibody testing via informed consent.

Antibody reactivity. Serum from Patient 1, in addition to serum from a cohort of APS1 patients (n=68) without lipodystrophy and normal control sera (n=54), including those patients described in Examples 1-10, was tested for autoantibodies specific to PLIN1 via RLBA (FIG. 15). Antibody reactivity to PLIN1 was confirmed using immunohistochemistry on mouse enteric tissue containing fat and colocalization of human sera and commercial antibody to PLIN1.

### REFERENCES CITED IN THIS DISCLOSURE

1. Ackermann, K., et al. (2006). Characterization of Human Melatonin Synthesis Using Autoptic Pineal Tissue. Endocrinology, 147(7), 3235-3242.
2. Adriaenssens, A. E., et al. (2019). Glucose-Dependent Insulinotropic Polypeptide Receptor-Expressing Cells in the Hypothalamus Regulate Food Intake. Cell Metabolism.
3. Ahonen, P., et al. (1990). Clinical Variation of Autoimmune Polyendocrinopathy-Candidiasis-Ectodermal Dystrophy (APECED) in a Series of 68 Patients. The New England Journal of Medicine, 322(26), 1829-1836.
4. Akoury, E., et al. (2015). NLRP7 and KHDC3L, the two maternal-effect proteins responsible for recurrent hydatidiform moles, co-localize to the oocyte cytoskeleton. Human Reproduction (Oxford, England), 30(1), 159-169.
5. Alanentalo, T., et al. (2006). Cloning and analysis of Nkx6.3 during CNS and gastrointestinal development. Gene Expression Patterns, 6(2), 162-170.
6. Alimohammadi, M., et al. (2008). Autoimmune polyendocrine syndrome type 1 and NALPS, a parathyroid autoantigen. The New England Journal of Medicine, 358(10), 1018-1028.
7. Alimohammadi, M., et al. (2009). Pulmonary autoimmunity as a feature of autoimmune polyendocrine syndrome type 1 and identification of KCNRG as a bronchial autoantigen. Proceedings of the National Academy of Sciences, 106(11), 4396-4401.
8. Bebbere, D., et al. (2016). The subcortical maternal complex: multiple functions for one biological structure? Journal of Assisted Reproduction and Genetics, 33(11), 1431-1438.
9. Berger, M. et al. (2015). Gai/o-coupled receptor signaling restricts pancreatic b-cell expansion. PNAS 112:2888-2893.
10. Berson, S. A., et al. (1956). Insulin-I¹³¹ metabolism in human subjects: Demonstration of insulin binding globulin in the circulation of insulin treated subjects. Journal of Clinical Investigation, 35(2), 170-190.
11. Betterle, C., et al. (2002). Autoimmune Adrenal Insufficiency and Autoimmune Polyendocrine Syndromes: Autoantibodies, Autoantigens, and Their Applicability in Diagnosis and Disease Prediction. Endocrine Reviews, 23(3), 327-364.
12. Bjork, E., et al. (1994). GAD Autoantibodies in IDDM, Stiff-Man Syndrome, and Autoimmune Polyendocrine Syndrome Type I Recognize Different Epitopes. Diabetes, 43(1), 161-165.
13. Brozzetti, A., et al. (2015). Autoantibody Response Against NALP5/MATER in Primary Ovarian Insufficiency and in Autoimmune Addison's Disease. The Journal of Clinical Endocrinology & Metabolism, 100(5), 1941-1948.

14. Bruserud, Ø., et al. (2016). A Longitudinal Follow-up of Autoimmune Polyendocrine Syndrome Type 1. The Journal of Clinical Endocrinology & Metabolism, 101(8), 2975-2983.
15. Cheng, M., & Anderson, M. S. (2018). Thymic tolerance as a key brake on autoimmunity. Nature Immunology, 19(7), 659-664.
16. Choi, H., et al. (2016). Testicular acid phosphatase induces odontoblast differentiation and mineralization. Cell and Tissue Research, 364(1), 95-103.
17. Clemente, M., et al. (1997). Cytochrome P450 1A2 Is a Hepatic Autoantigen in Autoimmune Polyglandular Syndrome Type 1 1. The Journal of Clinical Endocrinology & Metabolism, 82(5), 1353-1361.
18. Ekwall, 0., et al. (1998). Identification of tryptophan hydroxylase as an intestinal autoantigen. The Lancet, 352(9124), 279-283.
19. Ferré, E. M., et al. (2019). Lymphocyte-driven regional immunopathology in pneumonitis caused by impaired central immune tolerance. Science Translational Medicine, 11(495), eaav5597.
20. Ferré, E. M., et al. (2016). Redefined clinical features and diagnostic criteria in autoimmune polyendocrinopathy-candidiasis-ectodermal dystrophy. JCI Insight, 1(13), e88782.
21. Fishman, D., et al. (2017). Autoantibody Repertoire in APECED Patients Targets Two Distinct Subgroups of Proteins. Frontiers in Immunology, 8, 976.
22. Gehart, H., et al. (2019). Identification of Enteroendocrine Regulators by Real-Time Single-Cell Differentiation Mapping. Cell, 176(Dev. Dyn. 194 1992), 1158-1173.e16.
23. Goldspink, D. A., et al. (2018). Models and Tools for Studying Enteroendocrine Cells. Endocrinology, 159(12), 3874-3884.
24. Green, D. R., et al. (2019). Mapping the Tooth Enamel Proteome and Amelogenin Phosphorylation Onto Mineralizing Porcine Tooth Crowns. Frontiers in Physiology, 10, 925.
25. Hedstrand, H., et al. (2001). The Transcription Factors SOX9 and SOX10 Are Vitiligo Autoantigens in Autoimmune Polyendocrine Syndrome Type I. Journal of Biological Chemistry, 276(38), 35390-35395.
26. Holland, A. M., et al. (2002). Experimental control of pancreatic development and maintenance. Proceedings of the National Academy of Sciences, 99(19), 12236-12241.
27. Huhtaniemi, I., et al. (2018). Advances in the Molecular Pathophysiology, Genetics, and Treatment of Primary Ovarian Insufficiency. Trends in Endocrinology & Metabolism, 29(6), 400-419.
28. Husebye, E. S., et al. (1997). Autoantibodies against Aromatic 1-Amino Acid Decarboxylase in Autoimmune Polyendocrine Syndrome Type I 1. The Journal of Clinical Endocrinology & Metabolism, 82(1), 147-150.
29. Jasti, S., et al. (2012). The Autoimmune Regulator Prevents Premature Reproductive Senescence in Female Mice. Biology of Reproduction, 86(4), 110.
30. Kluger, N., et al. (2015). Gastrointestinal immunity against tryptophan hydroxylase-1, aromatic L-amino-acid decarboxylase, AIE-75, villin and Paneth cells in APECED. Clinical Immunology, 158(2), 212-220.
31. Kuroda, N., et al. (2005). Development of Autoimmunity against Transcriptionally Unrepressed Target Antigen in the Thymus of Aire-Deficient Mice. The Journal of Immunology, 174(4), 1862-1870.
32. Landegren, N., et al. (2016). Proteome-wide survey of the autoimmune target repertoire in autoimmune polyendocrine syndrome type 1. Nature Publishing Group, 6(1), 1-11.
33. Landegren, N., et al. (2015). Transglutaminase 4 as a prostate autoantigen in male subfertility. Science Translational Medicine, 7(292), 292ra101.
34. Larman, B. H., et al. (2011). Autoantigen discovery with a synthetic human peptidome. Nature Biotechnology, 29(6), 535 541.
35. Leonard, J. D., et al. (2017). Identification of Natural Regulatory T Cell Epitopes Reveals Convergence on a Dominant Autoantigen. Immunity, 47(1), 107-117.e8.
36. Li, L., et al. (2008). A Subcortical Maternal Complex Essential for Preimplantation Mouse Embryogenesis. Developmental Cell, 15(3), 416-425.
37. Liu, C., et al. (2016). ECAT1 is essential for human oocyte maturation and preimplantation development of the resulting embryos. Scientific Reports, 6(1), 38192.
38. Maclaren, N., et al. (2001). Autoimmune hypogonadism as part of an autoimmune polyglandular syndrome. Journal of the Society for Gynecologic Investigation, 8(1), S52-S54.
39. Mandel-Brehm, C., et al. (2019). Kelch-like Protein 11 Antibodies in Seminoma-Associated Paraneoplastic Encephalitis. New England Journal of Medicine, 381(1), 47-54.
40. Meager, A., et al. (2006). Anti-Interferon Autoantibodies in Autoimmune Polyendocrinopathy Syndrome Type 1. PLoS Medicine, 3(7), e289.
41. Meyer, S., et al. (2016). AIRE-Deficient Patients Harbor Unique High-Affinity Disease-Ameliorating Autoantibodies. Cell, 166(3), 582-595.
42. Moody, A. J., et al. (1984). The isolation and sequencing of human gastric inhibitory peptide (GIP). FEBS Letters, 172(2), 142-148.
43. Nelson, L. M. (2009). Clinical practice. Primary ovarian insufficiency. The New England Journal of Medicine, 360(6), 606-614.
44. O'Connor, D. T., et al. (1983). Chromogranin A: Immunohistology reveals its universal occurrence in normal polypeptide hormone producing endocrine glands. Life Sciences, 33(17), 1657-1663.
45. O'Donovan, B., et al. (2018). Exploration of Anti-Yo and Anti-Hu paraneoplastic neurological disorders by PhIP-Seq reveals a highly restricted pattern of antibody epitopes. bioRxiv 502187. Published in. final form as: O'Donovan, B., et al. (2020). High-resolution epitope mapping of anti-Hu and anti-Yo autoimmunity by programmable phage display, Brain Communications, 2 (2), fcaa059, doi.org/10.1093/braincomms/fcaa059.
46. Oftedal, B. E., et al. (2015). Dominant Mutations in the Autoimmune Regulator AIRE Are Associated with Common Organ-Specific Autoimmune Diseases. Immunity, 42(6), 1185-1196.
47. Ohsie, S., et al. (2009). A paucity of colonic enteroendocrine and/or enterochromaffin cells characterizes a subset of patients with chronic unexplained diarrhea/malabsorption. Human Pathology, 40(7), 1006-1014.
48. Otsuka, N., et al. (2011). Autoimmune Oophoritis with Multiple Molecular Targets Mitigated by Transgenic Expression of Mater. Endocrinology, 152(6), 2465 2473.
49. Patel, K. A., et al. (2017). Heterozygous RFX6 protein truncating variants are associated with MODY with reduced penetrance. Nature Communications, 8(1), 1-8.
50. Pederson, R. A., & McIntosh, C. H. (2016). Discovery of gastric inhibitory polypeptide and its subsequent fate: Personal reflections. Journal of Diabetes Investigation, 7(S1), 4-7.
51. Piccand, J., et al. (2014). Rfx6 Maintains the Functional Identity of Adult Pancreatic &beta; Cells. CELREP, 9(6), 2219-2232.
52. Piccand, J., et al. (2019). Rfx6 promotes the differentiation of peptide-secreting enteroendocrine cells while repressing genetic programs controlling serotonin production. Molecular Metabolism, 29, 24-39.
53. Pontynen, N., et al. (2006). Aire deficient mice do not develop the same profile of tissue-specific autoantibodies as APECED patients. Journal of Autoimmunity, 27(2), 96-104.
54. Rath, M. F., et al. (2016). Melatonin Synthesis: Acetylserotonin 0-Methyltransferase (ASMT) Is Strongly Expressed in a Subpopulation of Pinealocytes in the Male Rat Pineal Gland. Endocrinology, 157(5), 2028-2040.
55. Reddy, R., et al. (2012). Report of four new patients with protein-truncating mutations in C6orf221/KHDC3L and colocalization with NLRP7. European Journal of Human Genetics, 21(9), 957-964.
56. Rezaei, M., et al. (2016). Two novel mutations in the KHDC3L gene in Asian patients with recurrent hydatidiform mole. Human Genome Variation, 3(1), 16027.
57. Sansom, S. N., et al. (2014). Population and single-cell genomics reveal the Aire dependency, relief from Polycomb silencing, and distribution of self-antigen expression in thymic epithelia. Genome Research, 24(12), 1918-1931.
58. Schaum, N., et al. (2018). Single-cell transcriptomics of 20 mouse organs creates a Tabula Muris. Nature, 562(7727), 1-25.
59. Seymen, F., et al. (2016). Recessive Mutations in ACPT, Encoding Testicular Acid Phosphatase, Cause Hypoplastic Amelogenesis Imperfecta. The American Journal of Human Genetics, 99(5), 1199-1205.
60. Shum, A. K., et al. (2013). BPIFB1 is a lung-specific autoantigen associated with interstitial lung disease. Science Translational Medicine, 5(206), 206ra139 206ra139.
61. Shum, A. K., et al. (2009). Identification of an autoantigen demonstrates a link between interstitial lung disease and a defect in central tolerance. Science Translational Medicine, 1(9), 9ra20.
62. Sifuentes-Dominguez, L. F., et al. (2019). SCGN deficiency results in colitis susceptibility. ELife, 8, e49910.
63. Silva, C., et al. (2014). Autoimmune primary ovarian insufficiency. Autoimmunity Reviews, 13(4-5), 427-430.
64. Smith, C. E., et al. (2017). Defects in the acid phosphatase ACPT cause recessive hypoplastic amelogenesis imperfecta. European Journal of Human Genetics, 25(8), 1015.
65. Smith, S. B., et al. (2010). Rfx6 directs islet formation and insulin production in mice and humans. Nature, 463(7282), 775-780.
66. Soderbergh, A., et al. (2004). Prevalence and Clinical Associations of 10 Defined Autoantibodies in Autoimmune Polyendocrine Syndrome Type I. The Journal of Clinical Endocrinology & Metabolism, 89(2), 557-562.
67. Sorkina, E., et al. (2016). Progressive Generalized Lipodystrophy as a Manifestation of Autoimmune Polyglandular Syndrome Type 1. The Journal of Clinical Endocrinology & Metabolism, 101(4), 1344-1347.
68. Stoffers, D. A., et al. (1997). Pancreatic agenesis attributable to a single nucleotide deletion in the human IPF1 gene coding sequence. Nature Genetics, 15(1), 106-110.
69. Uhlen, M., et al. (2015). Tissue-based map of the human proteome. Science, 347(6220), 1260419.
70. Vazquez et al. (2020). Identification of novel, clinically correlated autoantigens in the monogenic autoimmune syndrome APS1 by proteome-wide PhIP-Seq. eLife, 9: e55053.
71. Velloso, L., et al. (1994). Autoantibodies against a novel 51 kDa islet antigen and glutamate decarboxylase isoforms in autoimmune polyendocrine syndrome type I. Diabetologia, 37(1), 61-69.
72. Virant-Klun, I., et al. (2016). Identification of Maturation-Specific Proteins by Single-Cell Proteomics of Human Oocytes. Molecular & Cellular Proteomics: MCP, 15(8), 2616-2627.
73. Wang, J., et al. (2006). Mutant Neurogenin-3 in Congenital Malabsorptive Diarrhea. The New England Journal of Medicine, 355(3), 270-280.
74. Wang, X., et al. (2018). Novel mutations in genes encoding subcortical maternal complex proteins may cause human embryonic developmental arrest. Reproductive BioMedicine Online, 36(6), 698-704.
75. Welt, C. K. (2008). Autoimmune Oophoritis in the Adolescent. Annals of the New York Academy of Sciences, 1135(1), 118-122.
76. Wolff, A., et al. (2013). Anti-Cytokine Autoantibodies Preceding Onset of Autoimmune Polyendocrine Syndrome Type I Features in Early Childhood. Journal of Clinical Immunology, 33(8), 1341-1348.
77. Yousef, G. M., et al. (2001). Molecular Cloning of a Novel Human Acid Phosphatase Gene (ACPT) That Is Highly Expressed in the Testis. Genomics, 74(3), 385-395.
78. Zhang, W., et al. (2019). KHDC3L mutation causes recurrent pregnancy loss by inducing genomic instability of human early embryonic cells. PLOS Biology, 17(10), e3000468.
79. Zhang, Y., et al. (2018). Transcriptome Landscape of Human Folliculogenesis Reveals Oocyte and Granulosa Cell Interactions. Molecular Cell, 72(6), 1-19.
80. Zhu, K., et al. (2015). Identification of a human subcortical maternal complex. Molecular Human Reproduction, 21(4), 320-329.
81. Ziegler, B., et al. (1996). Murine monoclonal glutamic acid decarboxylase (GAD)65 antibodies recognize autoimmune-associated GAD epitope regions targeted in patients with type 1 diabetes mellitus and Stiff-man syndrome. Acta Diabetologica, 33(3), 225-231.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments, such substitution is considered within the scope of the disclosure.

The examples presented herein are intended to illustrate potential and specific implementations of the invention. It can be appreciated that the examples are intended primarily for purposes of illustration for those skilled in the art. There may be variations to these diagrams or the operations described herein without departing from the spirit of the invention. For instance, in certain cases, method steps or operations may be performed or executed in differing order, or operations may be added, deleted or modified.

Different arrangements of the components depicted in the drawings or described above, as well as components and steps not shown or described are possible. Similarly, some features and sub-combinations are useful and may be employed without reference to other features and sub-combinations. Aspects and embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

While exemplary embodiments have been described in some detail, by way of example and for clarity of understanding, those of skill in the art will recognize that a variety of modification, adaptations, and changes may be employed. Hence, the scope of the present invention should be limited solely by the claims.

1. A method of detecting the presence of an autoantibody associated with autoimmune polyglandular syndrome type 1 (APS1) in a biological sample from a subject suspected to have APS1, or a subject diagnosed with APS1, comprising the steps of:
(a) contacting the biological sample with one or more antigenic polypeptides listed in Table 1, or a fragment of one or more of the antigenic polypeptides listed in Table 1; and
(b) detecting the presence of binding of the antigenic polypeptide or fragment thereof to an APS1 associated autoantibody in the biological sample.

2-5. (canceled)

6. The method of claim 1, wherein step (b) of detecting is performed by at least one of immunoprecipitation, microarray analysis, enzyme-linked immunosorbent assay (ELISA), or Western blot analysis.

7. The method of claim 1, wherein the antigenic polypeptide or fragment thereof is heterologously-expressed on the surface of a cell, a phage or a virus.

8. The method of claim 1, wherein the antigenic polypeptide or fragment thereof is expressed in a phage display or eukaryotic cell display library.

9. The method of claim 1, wherein the antigenic polypeptide or fragment thereof is an isolated, purified antigenic polypeptide or fragment thereof.

10. The method of claim 1, wherein the antigenic polypeptide or fragment thereof is an isolated, purified antigenic polypeptide or fragment thereof that is immobilized on a solid carrier.

11. The method of claim 1
wherein the RFX6 antigenic polypeptide or fragment thereof comprises one or more of the sequences of SEQ ID NOs: 31-37;
wherein the KHDC3L antigenic polypeptide or fragment thereof comprises one or more of the sequences of SEQ ID NOs: 38-40;
wherein the ACPT antigenic polypeptide or fragment thereof comprises the sequence of SEQ ID NO: 41; and/or
wherein the PLIN1 antigenic polypeptide or fragment thereof comprises one or more of the sequences of SEQ ID NOs: 42-46.

12. The method of claim 1, wherein the RFX6 antigenic polypeptide comprises the sequence of SEQ ID NO:24, the KHDC3L antigenic polypeptide comprises the sequence of SEQ ID NO: 13, the ACPT antigenic polypeptide comprises the sequence of SEQ ID NO: 29, and the PLIN1 antigenic polypeptide comprises the sequence of SEQ ID NO: 30.

13. The method of claim 1, wherein the biological sample is serum, plasma, cerebrospinal fluid, blood, or urine.

14. The method of claim 1, wherein the subject has been determined to have a loss of function mutation in the AIRE gene.

15. The method of claim 1, wherein the subject has one or more of nail dystrophy, hypoparathyroidism, keratoconjunctivitis, chronic mucocutaneous candidiasis, intestinal dysfunction, autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis.

16. The method of claim 1, wherein the subject has at least one of intestinal dysfunction or primary ovarian insufficiency.

17. The method of claim 1, wherein the presence of the binding of the antigenic polypeptide or fragment thereof in the biological sample to one or more of said RFX6 autoantibody, KHDC3L autoantibody, ACPT autoantibody, or PLIN1 autoantibody indicates that the subject has APS1.

18. The method of claim 1,
wherein the subject has or is at risk of developing diarrheal-type intestinal dysfunction, and wherein the RFX6 antigenic polypeptide or fragment thereof binds to said RFX6 autoantibody in the biological sample;
wherein the subject has or is at risk of developing premature ovarian failure, and wherein the KHDC3L antigenic polypeptide or fragment thereof binds to said KHDC3L autoantibody in the biological sample;
wherein the subject has or is at risk of developing dental enamel hypoplasia, and wherein the ACPT antigenic polypeptide or fragment thereof binds to said ACPT autoantibody in the biological sample; and/or
wherein the subject has or is at risk of developing lipodystrophy, and wherein the PLIN1 antigenic polypeptide or fragment thereof binds to said PLIN1 autoantibody in the biological sample.

19-20. (canceled)

21. A method of treating a subject having autoimmune polyglandular syndrome type 1(APS1), comprising the steps of:
(a) detecting the presence an APS1 associated autoantibody that binds specifically to an antigenic polypeptide or fragment thereof listed in Table 1 in a biological sample from a subject using the method of claim 1, wherein detecting one or both of the autoantibodies in the biological sample indicates that the subject has APS1; and
(b) administering to the subject an immunosuppressive therapy.

22. The method of claim 21, wherein the subject has one or more of nail dystrophy, hypoparathyroidism, keratoconjunctivitis, chronic mucocutaneous candidiasis, intestinal dysfunction, autoimmune hepatitis, primary ovarian insufficiency, hypertension, hypothyroidism, vitamin B12 deficiency, diabetes mellitus, Sjogren's-like syndrome, growth hormone deficiency, adrenal insufficiency, dental enamel hypoplasia, testicular failure, tubulointerstital nephritis, hypopituitarism, vitiligo, gastritis, urticarial eruption, alopecia, asplenia, lipodystrophy, or pneumonitis.

23. The method of claim 21, wherein the subject has at least one of intestinal dysfunction, primary ovarian insufficiency, dental enamel hypoplasia, or lipodystrophy.

24. The method of claim 21,
wherein the subject has diarrheal-type intestinal dysfunction, and wherein the presence or absence of a RFX6 autoantibody is detected in the biological sample;
wherein the subject has primary ovarian insufficiency, and wherein the presence or absence of a KHDC3L autoantibody is detected in the biological sample;
wherein the subject has dental enamel hypoplasia, and wherein the presence or absence of an ACPT autoantibody is detected in the biological sample; and/or
wherein the subject has lipodystrophy, and wherein the presence or absence of a PLIN1 autoantibody is detected in the biological sample.

25-26. (canceled)

27. The method of claim 21, wherein the subject has been determined to have a loss of function mutation in the AIRE gene.

28. The method of claim 21, wherein the immunosuppressive therapy comprises at least one of an immunosuppressant drug, intravenous immunoglobulin administration, plasma exchange plasmapheresis, immunoadsorption, or oral administration of the antigenic polypeptide or immunogenic fragments thereof.

29. The method of claim 21, wherein the subject is also administered:
(i) a therapy for diarrheal-type intestinal dysfunction if a RFX6 autoantibody is detected in the biological sample;
(ii) a therapy for primary ovarian insufficiency if a KHDC3L autoantibody is detected in the biological sample;
(iii) a therapy for dental enamel hypoplasia if an ACPT autoantibody is detected in the biological sample; and/or
(iv) a therapy for lipodystrophy if a PLIN1 autoantibody is detected in the biological sample.


SEQUENCE LISTING (46 sequences)

SEQ ID NO: 1 - protein, 575 aa, Homo Sapiens
MQSPWKILTVAPLFLLLSLQSSASPANDDQSRPSLSNGHTCVGCVLVVSVIEQLAQVHNSTVQASMERLCSYLPEKLFLKTTCYLVIDKFGSDIIKLLSADMNADVVCHTLEFCKQNTGQPLCHLYPLPKETWKFTLQKARQIVKKSPILKYSRSGSDICSLPVLAKICQKIKLAMEQSVPFKDVDSDKYSVFPTLRGYHWRGRDCNDSDESVYPGRRPNNWDVHQDSNCNGIWGVDPKDGVPYEKKFCEGSQPRGIILLGDSAGAHFHISPEWITASQMSLNSFINLPTALTNELDWPQLSGATGFLDSTVGIKEKSIYLRLWKRNHCNHRDYQNISRNGASSRNLKKFIESLSRNKVLDYPAIVIYAMIGNDVCSGKSDPVPAMTTPEKLYSNVMQTLKHLNSHLPNGSHVILYGLPDGTFLWDNLHNRYHPLGQLNKDMTYAQLYSFLNCLQVSPCHGWMSSNKTLRTLTSERAEQLSNTLKKIAASEKFTNFNLFYMDFAFHEIIQEWQKRGGQPWQLIEPVDGFHPNEVALLLLADHFWKKVQLQWPQILGKENPFNPQIKQVFGDQGGH

SEQ ID NO: 2 - protein, 201 aa, Homo Sapiens
MYTLLSGLYKYMFQKDEYCILILGLDNAGKTTFLEQSKTRFNKNYKGMSLSKITTTVGLNIGTVDVGKARLMFWDLGGQEELQSLWDKYYAECHGVIYVIDSTDEERLAESKQAFEKVVTSEALCGVPVLVLANKQDVETCLSIPDIKTAFSDCTSKIGRRDCLTQACSALTGKGVREGIEWMVKCVVRNVHRPPRQRDIT

SEQ ID NO: 3 - protein, 414 aa, Homo Sapiens
MVLGKVKSLTISFDCLNDSNVPVYSSGDTVSGRVNLEVTGEIRVKSLKIHARGHAKVRWTESRNAGSNTAYTQNYTEEVEYFNHKDILIGHERDDDNSEEGFHTIHSGRHEYAFSFELPQTPLATSFEGRHGSVRYWVKAELHRPWLLPVKLKKEFTVFEHIDINTPSLLSPQAGTKEKTLCCWFCTSGPISLSAKIERKGYTPGESIQIFAEIENCSSRMVVPKAAIYQTQAFYAKGKMKEVKQLVANLRGESLSSGKTETWNGKLLKIPPVSPSILDCSIIRVEYSLMVYVDIPGAMDLFLNLPLVIGTIPLHPFGSRTSSVSSQCSMNMNWLSLSLPERPEAPPSYAEVVTEEQRRNNLAPVSACDDFERALQGPLFAYIQEFRFLPPPLYSEIDPNPDQSADDRPSCPSR

SEQ ID NO: 4 - protein, 194 aa, Homo Sapiens
MAKQKRKVPEVTEKKNKKLKKASAEGPLLGPEAAPSGEGAGSKGEAVLRPGLDAEPELSPEEQRVLERKLKKERKKEERQRLREAGLVAQHPPARRSGAELALDYLCRWAQKHKNWRFQKTRQTWLLLHMYDSDKVPDEHFSTLLAYLEGLQGRARELTVQKAEALMRELDEEGSDPPLPGRAQRIRQVLQLLS

SEQ ID NO: 5 - protein, 78 aa, Homo Sapiens
MSEVLPYGDEKLSPYGDGGDVGQIFSCRLQDTNNFFGAGQNKRPPKLGQIGRSKRVVIEDDRIDDVLKNMTDKAPPGV

SEQ ID NO: 6 - protein, 298 aa, Homo Sapiens
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 7 - protein, 744 aa, Homo Sapiens
MSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQAYSAYPHSPGQVITGCQLLVYNYQEYPTYPDSAFQVTTGYQLPVYNYQPFPAYPRSPFQVTAGYQLPVYNYQAFPAYPNSPFQVATGYQFPVYNYQPFPAYPSSPFQVTAGYQLPVYNYQAFPAYPNSPFQVATGYQFPVYNYQAFPAYPNSPVQVTTGYQLPVYNYQAFPAYPSSPFQVTTGYQLPVYNYQAFPAYPNSAVQVTTGYQFHVYNYQMPPQCPVGEQRRNLWTEAYKWWYLVCLIQRRD

SEQ ID NO: 8 - protein, 579 aa, Homo Sapiens
MSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQAYSAYPHSPGQVITGCQLLVYNYQEYPTYPDSAFQVTTGYQLPVYNYQPFPAYPRSPFQVTAGYQLPVYNYQAFPAYPNSPFQVATGYQFPVYNYQPFPAYPSSPFQVTAGYQLPVYNYQAFPAYPNSPFQVATGYQFPVYNYQAFPAYPNSPVQVTTGYQLPVYNYQAFPAYPSSPFQVTTGYQLPVYNYQAFPAYPNSAVQVTTGYQFHVYNYQMPPQCPVGEQRRNLWTEAYKWWYLVCLIQRRD

SEQ ID NO: 9 - protein, 326 aa, Homo Sapiens
MALSGSTPAPCWEEDECLDYYGMLSLHRMFEVVGGQLTECELELLAFLLDEAPGAAGGLARARSGLELLLELERRGQCDESNLRLLGQLLRVLARHDLLPHLARKRRRPVSPERYSYGTSSSSKRTEGSCRRRRQSSSSANSQQGQWETGSPPTKRQRRSRGRPSGGARRRRRGAPAAPQQQSEPARPSSEGKVTCDIRLRVRAEYCEHGPALEQGVASRRPQALARQLDVFGQATAVLRSRDLGSVVCDIKFSELSYLDAFWGDYLSGALLQALRGVFLTEALREAVGREAVRLLVSVDEADYEAGRRRLLLMEEEGGRRPTEAS

SEQ ID NO: 10 - protein, 327 aa, Homo Sapiens
MAAAAAMAEQESARNGGRNRGGVQRVEGKLRASVEKGDYYEAHQMYRTLFFRYMSQSKHTEARELMYSGALLFFSHGQQNSAADLSMLVLESLEKAEVEVADELLENLAKVFSLMDPNSPERVTFVSRALKWSSGGSGKLGHPRLHQLLALTLWKEQNYCESRYHFLHSADGEGCANMLVEYSTSRGFRSEVDMFVAQAVLQFLCLKNKSSASVVFTTYTQKHPSIEDGPPFVEPLLNFIWFLLLAVDGGKLTVFTVLCEQYQPSLRRDPMYNEYLDRIGQLFFGVPPKQTSSYGGLLGNLLTSLMGSSEQEDGEESPSDGSPIELD

SEQ ID NO: 11 - protein, 153 aa, Homo Sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 12 - protein, 354 aa, Homo Sapiens
MKSLLLLVLISICWADHLSDNYTLDHDRAIHIQAENGPHLLVEAEQAKVFSHRGGNVTLPCKFYRDPTAFGSGIHKIRIKWTKLTSDYLKEVDVFVSMGYHKKTYGGYQGRVFLKGGSDSDASLVITDLTLEDYGRYKCEVIEGLEDDTVVVALDLQGVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKILGYDRCDAGWLADGSVRYPISRPRRRCSPTEAAVRFVGFPDKKHKLYGVYCFRAYN

SEQ ID NO: 13 - protein, 217 aa, Homo Sapiens
MDAPRRFPTLVQLMQPKAMPVEVLGHLPKRFSWFHSEFLKNPKVVRLEVWLVEKIFGRGGERIPHVQGMSQILIHVNRLDPNGEAEILVFGRPSYQEDTIKMIMNLADYHRQLQAKGSGKALAQDVATQKAETQRSSIEVREAGTQRSVEVREAGTQRSVEVQEVGTQGSPVEVQEAGTQQSLQAANKSGTQRSPEAASKAVTQRFREDARDPVTRL

SEQ ID NO: 14 - protein, 1032 aa, Homo Sapiens
MAFTNYSSLNRAQLTFEYLHTNSTTHEFLFGALAELVDNARDADATRIDIYAERREDLRGGFMLCFLDDGAGMDPSDAASVIQFGKSAKRTPESTQIGQYGNGLKSGSMRIGKDFILFTKKEDTMTCLFLSRTFHEEEGIDEVIVPLPTWNARTREPVTDNVEKFAIETELIYKYSPFRTEEEVMTQFMKIPGDSGTLVIIFNLKLMDNGEPELDIISNPRDIQMAETSPEGTKPERRSFRAYAAVLYIDPRMRIFIHGHKVQTKRLSCCLYKPRMYKYTSSRFKTRAEQEVKKAEHVARIAEEKAREAESKARTLEVRLGGDLTRDSRVMLRQVQNRAITLRREADVKKRIKEAKQRALKEPKELNFVFGVNIEHRDLDGMFIYNCSRLIKMYEKVGPQLEGGMACGGVVGVVDVPYLVLEPTHNKQDFADAKEYRHLLRAMGEHLAQYWKDIAIAQRGIIKFWDEFGYLSANWNQPPSSELRYKRRRAMEIPTTIQCDLCLKWRTLPFQLSSVEKDYPDTWVCSMNPDPEQDRCEASEQKQKVPLGTFRKDMKTQEEKQKQLTEKIRQQQEKLEALQKTTPIRSQADLKKLPLEVTTRPSTEEPVRRPQRPRSPPLPAVIRNAPSRPPSLPTPRPASQPRKAPVISSTPKLPALAAREEASTSRLLQPPEAPRKPANTLVKTASRPAPLVQQLSPSLLPNSKSPREVPSPKVIKTPVVKKTESPIKLSPATPSRKRSVAVSDEEEVEEEAERRKERCKRGRFVVKEEKKDSNELSDSAGEEDSADLKRAQKDKGLHVEVRVNREWYTGRVTAVEVGKHVVRWKVKFDYVPTDTTPRDRWVEKGSEDVRLMKPPSPEHQSLDTQQEGGEEEVGPVAQQAIAVAEPSTSECLRIEPDTTALSTNHETIDLLVQILRNCLRYFLPPSFPISKKQLSAMNSDELISFPLKEYFKQYEVGLQNLCNSYQSRADSRAKASEESLRTSERKLRETEEKLQKLRTNIVALLQKVQEDIDINTDDELDAYIEDLITKGD

SEQ ID NO: 15 - protein, 5762 aa, Homo Sapiens
MGAPSACRTLVLALAAMLVVPQAETQGPVEPSWENAGHTMDGGAPTSSPTRRVSFVPPVTVFPSLSPLNPAHNGRVCSTWGDFHYKTFDGDVFRFPGLCNYVFSEHCRAAYEDFNVQLRRGLVGSRPVVTRVVIKAQGLVLEASNGSVLINGQREELPYSRTGLLVEQSGDYIKVSIRLVLTFLWNGEDSALLELDPKYANQTCGLCGDFNGLPAFNEFYAHNARLTPLQFGNLQKLDGPTEQCPDPLPLPAGNCTDEEGICHRTLLGPAFAECHALVDSTAYLAACAQDLCRCPTCPCATFVEYSRQCAHAGGQPRNWRCPELCPRTCPLNMQHQECGSPCTDTCSNPQRAQLCEDHCVDGCFCPPGTVLDDITHSGCLPLGQCPCTHGGRTYSPGTSFNTTCSSCTCSGGLWQCQDLPCPGTCSVQGGAHISTYDEKLYDLHGDCSYVLSKKCADSSFTVLAELRKCGLTDNENCLKAVTLSLDGGDTAIRVQADGGVFLNSIYTQLPLSAANITLFTPSSFFIVVQTGLGLQLLVQLVPLMQVFVRLDPAHQGQMCGLCGNFNQNQADDFTALSGVVEATGAAFANTWKAQAACANARNSFEDPCSLSVENENYARHWCSRLTDPNSAFSRCHSIINPKPFHSNCMFDTCNCERSEDCLCAALSSYVHACAAKGVQLSDWRDGVCTKYMQNCPKSQRYAYVVDACQPTCRGLSEADVTCSVSFVPVDGCTCPAGTFLNDAGACVPAQECPCYAHGTVLAPGEVVHDEGAVCSCTGGKLSCLGASLQKSTGCAAPMVYLDCSNSSAGTPGAECLRSCHTLDVGCFSTHCVSGCVCPPGLVSDGSGGCIAEEDCPCVHNEATYKPGETIRVDCNTCTCRNRRWECSHRLCLGTCVAYGDGHFITFDGDRYSFEGSCEYILAQDYCGDNTTHGTFRIVTENIPCGTTGTTCSKAIKLFVESYELILQEGTFKAVARGPGGDPPYKIRYMGIFLVIETHGMAVSWDRKTSVFIRLHQDYKGRVCGLCGNFDDNAINDFATRSRSVVGDALEFGNSWKLSPSCPDALAPKDPCTANPFRKSWAQKQCSILHGPTFAACRSQVDSTKYYEACVNDACACDSGGDCECFCTAVAAYAQACHDAGLCVSWRTPDTCPLFCDFYNPHGGCEWHYQPCGAPCLKTCRNPSGHCLVDLPGLEGCYPKCPPSQPFFNEDQMKCVAQCGCYDKDGNYYDVGARVPTAENCQSCNCTPSGIQCAHSLEACTCTYEDRTYSYQDVIYNTTDGLGACLIAICGSNGTIIRKAVACPGTPATTPFTFTTAWVPHSTTSPALPVSTVCVREVCRWSSWYNGHRPEPGLGGGDFETFENLRQRGYQVCPVLADIECRAAQLPDMPLEELGQQVDCDRMRGLMCANSQQSPPLCHDYELRVLCCEYVPCGPSPAPGTSPQPSLSASTEPAVPTPTQTTATEKTTLWVTPSIRSTAALTSQTGSSSGPVTVTPSAPGTTTCQPRCQWTEWFDEDYPKSEQLGGDVESYDKIRAAGGHLCQQPKDIECQAESFPNWTLAQVGQKVHCDVHFGLVCRNWEQEGVFKMCYNYRIRVLCCSDDHCRGRATTPPPTTELETATTTTTQALFSTPQPTSSPGLTRAPPASTTAVPTLSEGLTSPRYTSTLGTATTGGPTTPAGSTEPTVPGVATSTLPTRSALPGTTGSLGTWRPSQPPTLAPTTMATSRARPTGTASTASKEPLTTSLAPTLTSELSTSQAETSTPRTETTMSPLTNTTTSQGTTRCQPKCEWTEWFDVDFPTSGVAGGDMETFENIRAAGGKMCWAPKSIECRAENYPEVSIDQVGQVLTCSLETGLTCKNEDQTGRFNMCFNYNVRVLCCDDYSHCPSTPATSSTATPSSTPGTTWILTKPTTTATTTASTGSTATPTSTLRTAPPPKVLTTTATTPTVTSSKATPSSSPGTATALPALRSTATTPTATSVTPIPSSSLGTTWTRLSQTTTPTATMSTATPSSTPETAHTSTVLTATATTTGATGSVATPSSTPGTAHTTKVPTTTTTGFTATPSSSPGTALTPPVWISTTTTPTTRGSTVTPSSIPGTTHTATVLTTTTTTVATGSMATPSSSTQTSGTPPSLTTTATTITATGSTTNPSSTPGTTPIPPVLTTTATTPAATSNTVTPSSALGTTHTPPVPNTMATTHGRSLPPSSPHTVRTAWTSATSGILGTTHITEPSTVTSHTLAATTGTTQHSTPALSSPHPSSRTTESPPSPGTTTPGHTTATSRTTATATPSKTRTSTLLPSSPTSAPITTVVTMGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTAMPSSTPGTTWILTELTTTATTTESTGSTATPSSTPGTTWILTEPSTTATVTVPTGSTATASSTQATAGTPHVSTTATTPTVTSSKATPFSSPGTATALPALRSTATTPTATSFTAIPSSSLGTTWTRLSQTTTPTATMSTATPSSTPETVHTSTVLTTTATTTGATGSVATPSSTPGTAHTTKVLTTTTTGFTATPSSSPGTARTLPVWISTTTTPTTRGSTVTPSSIPGTTHTPTVLTTTTTTVATGSMATPSSSTQTSGTPPSLTTTATTITATGSTTNPSSTPGTTPIPPVLTTTATTPAATSSTVTPSSALGTTHTPPVPNTTATTHGRSLSPSSPHTVRTAWTSATSGTLGTTHITEPSTGTSHTPAATTGTTQHSTPALSSPHPSSRTTESPPSPGTTTPGHTRATSRTTATATPSKTRTSTLLPSSPTSAPITTVVTMGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTATPSSTPGTTWILTEQTTAATTTATTGSTAIPSSTPGTAPPPKVLTSTATTPTATSSKATSSSSPRTATTLPVLTSTATKSTATSFTPIPSFTLGTTGTLPEQTTTPMATMSTIHPSSTPETTHTSTVLTTKATTTRATSSMSTPSSTPGTTWILTELTTAATTTAATGPTATPSSTPGTTWILTEPSTTATVTVPTGSTATASSTRATAGTLKVLTSTATTPTVISSRATPSSSPGTATALPALRSTATTPTATSVTAIPSSSLGTAWTRLSQTTTPTATMSTATPSSTPETVHTSTVLTTTTTTTRATGSVATPSSTPGTAHTTKVPTTTTTGFTATPSSSPGTALTPPVWISTTTTPTTRGSTVTPSSIPGTTHTATVLTTTTTTVATGSMATPSSSTQTSGTPPSLTTTATTITATGSTTNPSSTPGTTPIPPVLTTTATTPAATSSTVTPSSALGTTHTPPVPNTTATTHGRSLPPSSPHTVRTAWTSATSGILGTTHITEPSTVTSHTPAATTSTTQHSTPALSSPHPSSRTTESPPSPGTTTPGHTRGTSRTTATATPSKTRTSTLLPSSPTSAPITTVVTTGCEPQCAWSEWLDYSYPMPGPSGGDFDTYSNIRAAGGAVCEQPLGLECRAQAQPGVPLRELGQVVECSLDFGLVCRNREQVGKFKMCFNYEIRVFCCNYGHCPSTPATSSTATPSSTPGTTWILTKLTTTATTTESTGSTATPSSTPGTTWILTEPSTTATVTVPTGSTATASSTQATAGTPHVSTTATTPTVTSSKATPFSSPGTATALPALRSTATTPTATSFTAIPSSSLGTTWTRLSQTTTPTATMSTATPSSTPETAHTSTVLTTTATTTRATGSVATPSSTPGTAHTTKVPTTTTTGFTVTPSSSPGTARTPPVWISTTTTPTTSGSTVTPSSVPGTTHTPTVLTTTTTTVATGSMATPSSSTQTSGTPPSLITTATTITATGSTTNPSSTPGTTPIPPVLTTTATTPAATSSTVTPSSALGTTHTPPVPNTTATTHGRSLSPSSPHTVRTAWTSATSGTLGTTHITEPSTGTSHTPAATTGTTQHSTPALSSPHPSSRTTESPPSPGTTTPGHTTATSRTTATATPSKTRTSTLLPSSPTSAPITTVVTTGCEPQCAWSEWLDYSYPMPGPSGGDFDTYSNIRAAGGAVCEQPLGLECRAQAQPGVPLGELGQVVECSLDFGLVCRNREQVGKFKMCFNYEIRVFCCNYGHCPSTPATSSTAMPSSTPGTTWILTELTTTATTTASTGSTATPSSTPGTAPPPKVLTSPATTPTATSSKATSSSSPRTATTLPVLTSTATKSTATSVTPIPSSTLGTTGTLPEQTTTPVATMSTIHPSSTPETTHTSTVLTTKATTTRATSSTSTPSSTPGTTWILTELTTAATTTAATGPTATPSSTPGTTWILTELTTTATTTASTGSTATPSSTPGTTWILTEPSTTATVTVPTGSTATASSTQATAGTPHVSTTATTPTVTSSKATPSSSPGTATALPALRSTATTPTATSFTAIPSSSLGTTWTRLSQTTTPTATMSTATPSSTPETVHTSTVLTATATTTGATGSVATPSSTPGTAHTTKVPTTTTTGFTATPSSSPGTALTPPVWISTTTTPTTTTPTTSGSTVTPSSIPGTTHTARVLTTTTTTVATGSMATPSSSTQTSGTPPSLTTTATTITATGSTTNPSSTPGTTPITPVLTSTATTPAATSSKATSSSSPRTATTLPVLTSTATKSTATSFTPIPSSTLWTTWTVPAQTTTPMSTMSTIHTSSTPETTHTSTVLTTTATMTRATNSTATPSSTLGTTRILTELTTTATTTAATGSTATLSSTPGTTWILTEPSTIATVMVPTGSTATASSTLGTAHTPKVVTTMATMPTATASTVPSSSTVGTTRTPAVLPSSLPTFSVSTVSSSVLTTLRPTGFPSSHFSTPCFCRAFGQFFSPGEVIYNKTDRAGCHFYAVCNQHCDIDRFQGACPTSPPPVSSAPLSSPSPAPGCDNAIPLRQVNETWTLENCTVARCVGDNRVVLLDPKPVANVTCVNKHLPIKVSDPSQPCDFHYECECICSMWGGSHYSTFDGTSYTFRGNCTYVLMREIHARFGNLSLYLDNHYCTASATAAAARCPRALSIHYKSMDIVLTVTMVHGKEEGLILFDQIPVSSGFSKNGVLVSVLGTTTMRVDIPALGVSVTFNGQVFQARLPYSLFHNNTEGQCGTCTNNQRDDCLQRDGTTAASCKDMAKTWLVPDSRKDGCWAPTGTPPTASPAAPVSSTPTPTPCPPQPLCDLMLSQVFAECHNLVPPGPFFNACISDHCRGRLEVPCQSLEAYAELCRARGVCSDWRGATGGLCDLTCPPTKVYKPCGPIQPATCNSRNQSPQLEGMAEGCFCPEDQILFNAHMGICVQACPCVGPDGFPKFPGERWVSNCQSCVCDEGSVSVQCKPLPCDAQGQPPPCNRPGFVTVTRPRAENPCCPETVCVCNTTTCPQSLPVCPPGQESICTQEEGDCCPTFRCRPQLCSYNGTFYGVGATFPGALPCHMCTCLSGDTQDPTVQCQEDACNNTTCPQGFEYKRVAGQCCGECVQTACLTPDGQPVQLNETWVNSHVDNCTVYLCEAEGGVHLLTPQPASCPDVSSCRGSLRKTGCCYSCEEDSCQVRINTTILWHQGCETEVNITFCEGSCPGASKYSAEAQAMQHQCTCCQERRVHEETVPLHCPNGSAILHTYTHVDECGCTPFCVPAPMAPPHTRGFPAQEATAV

SEQ ID NO: 16 - protein, 265 aa, Homo Sapiens
MESNLQGTFLLNNTPLAQFPEMKAPVCQYSVQNSFYKLSPPGLGPQLAAGTPHGITDILSRPVAAPNNSLLSGYPHVAGFGGLSSQGVYYSPQVGNFSKAGNEYPTRTRNCWADTGQDWRGGRQCSNTPDPLSDSIHKKKHTRPTFTGHQIFALEKTFEQTKYLAGPERARLAYSLGMTESQVKVWFQNRRTKWRKKSALEPSSSTPRAPGGAGAGAGGDRAPSENEDDEYNKPLDPDSDDEKIRLLLRKHRAAFSVLSLGAHSV

SEQ ID NO: 17 - protein, 178 aa, Homo Sapiens
MPKAKGKTRRQKFGYSVNRKRLNRNARRKAAPRIECSHIRHAWDHAKSVRQNLAEMGLAVDPNRAVPLRKRKVKAMEVDIEERPKELVRKPYVLNDLEAEASLPEKKGNTLSRDLIDYVRYMVENHGEDYKAMARDEKNYYQDTPKQIRSKINVYKRFYPAEWQDFLDSLQKRKMEVE

SEQ ID NO: 18 - protein, 254 aa, Homo Sapiens [sequence omitted]

SEQ ID NO: 19 - protein, 252 aa, Homo Sapiens
MESEMETQSARAEEGFTQVTRKGGRRAKKRQAEQLSAAGEGGDAGRMDTEEARPAKRPVFPPLCGDGLLSGKEETRKIPVPANRYTPLKENWMKIFTPIVEHLGLQIRFNLKSRNVEIRTCKETKDVSALTKAADFVKAFILGFQVEDALALIRLDDLFLESFEITDVKPLKGDHLSRAIGRIAGKGGKTKFTIENVTRTRIVLADVKVHILGSFQNIKMARTAICNLILGNPPSKVYGNIRAVASRSADRF

SEQ ID NO: 20 - protein, 963 aa, Homo Sapiens
MLSGERKEGGSPRFGKLHLPVGLWINSPRKQLAKLGRRWPSAASVKSSSSDTGSRSSEPLPPPPPHVELRRVGAVKAAGGASGSRAKRISQLFRGSGTGTTGSSGAGGPGTPGGAQRWASEKKLPELAAGVAPEPPLATRATAPPGVLKIFGAGLASGANYKSVLATARSTARELVAEALERYGLAGSPGGGPGESSCVDAFALCDALGRPAAAGVGSGEWRAEHLRVLGDSERPLLVQELWRARPGWARRFELRGREEARRLEQEAFGAADSEGTGAPSWRPQKNRSRAASGGAALASPGPGTGSGAPAGSGGKERSENLSLRRSVSELSLQGRRRRQQERRQQALSMAPGAADAQIGTADPGDFDQLTQCLIQAPSNRPYFLLLQGYQDAQDFVVYVMTREQHVFGRGGNSSGRGGSPAPYVDTFLNAPDILPRHCTVRAGPEHPAMVRPSRGAPVTHNGCLLLREAELHPGDLLGLGEHFLFMYKDPRTGGSGPARPPWLPARPGATPPGPGWAFSCRLCGRGLQERGEALAAYLDGREPVLRFRPREEEALLGEIVRAAAAGSGDLPPLGPATLLALCVQHSARELELGHLPRLLGRLARLIKEAVWEKIKEIGDRQPENHPEGVPEVPLTPEAVSVELRPLMLWMANTTELLSFVQEKVLEMEKEADQEDPQLCNDLELCDEAMALLDEVIMCTFQQSVYYLTKTLYSTLPALLDSNPFTAGAELPGPGAELGAMPPGLRPTLGVFQAALELTSQCELHPDLVSQTFGYLFFFSNASLLNSLMERGQGRPFYQWSRAVQIRTNLDLVLDWLQGAGLGDIATEFFRKLSMAVNLLCVPRTSLLKASWSSLRTDHPTLTPAQLHHLLSHYQLGPGRGPPAAWDPPPAEREAVDTGDIFESFSSHPPLILPLGSSRLRLTGPVTDDALHRELRRLRRLLWDLEQQELPANYRHGPPVATSP

SEQ ID NO: 21 - protein, 392 aa, Homo Sapiens
MVEADRPGKLFIGGLNLETDEKALEAEFGKYGRIVEVLLMKDRETNKSRGFAFVTFESPADAKAAARDMNGKSLDGKAIKVAQATKPAFESSRRGPPPPRSRGRPRFLRGTRGGGGGPRRSPSRGGPDDDGGYTADFDLRPSRAPMPMKRGPPPRRVGPPPKRAAPSGPARSSGGGMRGRALAVRGRDGYSGPPRREPLPPRRDPYLGPRDEGYSSRDGYSSRDYREPRGFAPSPGEYTHRDYGHSSVRDDCPLRGYSDRDGYGGRDRDYGDHLSRGSHREPFESYGELRGAAPGRGTPPSYGGGGRYEEYRGYSPDAYSGGRDSYSSSYGRSDRYSRGRHRVGRPDRGLSLSMERGCPPQRDSYSRSGCRVPRGGGRLGGRLERGGGRSRY

SEQ ID NO: 22 - protein, 1067 aa, Homo Sapiens
MMEADRPEKLFIGGLNLKTDEKALKAEFGKYGHIIKVFLMKDRKTNKSRGFAFVTFESPADAKAAARDMNGKYLDGKAIMVAQTIKPAFKSSRWVPPTPGSGSRSRFSHRTRGGGSSPQRPPSQGRPDDGRGYAGYFDLWPYRAPMPRKRGPPPRHWASPPHKRATPSSLAHSVGCGMRGKAPTVSGQDGYSGLQPRRWAGPPHKRAVPRSSLARIGGSGMPGKAPAVWGQDGYSGPRVREPLPPCRDPGDFVPALRDYSRRYYGHSSVPDYRPLRGDGNQNGYRGRDHEYTDHPSKGSYREPLKSYGGPCGAAPVWGTPPSYGGGCRYEEYQGNSPDACSEGRSSEALPVVLPDAYSRDHSPKAYSGGRSSSSNGYSRSDRYGEEGCYEEYRGRSPDAHSGGRNSSSNSYGQSHHYGGEGRYEEYRGRSHEARSGGRSTDAHSRGRSDDAYSGGHDSSSWSDCCGGGGRYEEYQGRSLDANSGGCSPEAYSGGHDNSSWSDRYGVGGHYEENRGHSLDANSGGRSPDTHSGGHSSSSNSYGQSHRYGGEGRYEYRGRSHDAHSGGCSADAYSGGHDSSSQSNRYGGGGCYEEYRGRSLDANSGGRSPNAYSGGHDSSSWSHRYGGGGRYEEYRGRSLDANSGGRSPDAYSGGHDSSGQSNCYGGGGRYEEYRGRLLDANSGGRSPDAYSGGHDSSSQSNRYGGGGRYEEYRGHSLDANSGGRSPDTYSRGHDSSSQSDHYGGGGRSLDANSSGRLPDAYSGGHDSSSRSHRYGGGGRYEEYRGRSLDANSGGRSPNAYSGGHNSSSRNDPCRGGGRYEENRGHSLDANSGGHSPNAYSGGRDSSSNSYDRSHRYGGGGHYEEYRGRSHDTHSRGRSPDAHSGDHYTEAYSRGRDSFSNSYGRSDHYGRGGCYEEYQGRSPNAYGGGRGLNSSNNSHGRSHRYGGGGRYEEYRGPSPDAHSGGRDSSIKSYGLSDRYGGGGHYEEYQGSLPDAYSGDHDRSSNSYGRSDRYSRGRDRVGRPDRGLPLPMETGSPPLHDSYSRSGCRVPRGGGRQGGRFERGEGRSRY

SEQ ID NO: 23 - protein, 735 aa, Homo Sapiens
MHCGLLEEPDMDSTESWIERCLNESENKRYSSHTSLGNVSNDENEEKENNRASKPHSTPATLQWLEENYEIAEGVCIPRSALYMHYLDFCEKNDTQPVNAASFGKIIRQQFPQLTTRRLGTRGQSKYHYYGIAVKESSQYYDVMYSKKGAAWVSETGKKEVSKQTVAYSPRSKLGTLLPEFPNVKDLNLPASLPEEKVSTFIMMYRTHCQRILDTVIRANFDEVQSFLLHFWQGMPPHMLPVLGSSTVVNIVGVCDSILYKAISGVLMPTVLQALPDSLTQVIRKFAKQLDEWLKVALHDLPENLRNIKFELSRRFSQILRRQTSLNHLCQASRTVIHSADITFQMLEDWRNVDLNSITKQTLYTMEDSRDEHRKLITQLYQEFDHLLEEQSPIESYIEWLDTMVDRCVVKVAAKRQGSLKKVAQQFLLMWSCFGTRVIRDMTLHSAPSFGSFHLIHLMFDDYVLYLLESLHCQERANELMRAMKGEGSTAEVREEIILTEAAAPTPSPVPSFSPAKSATSVEVPPPSSPVSNPSPEYTGLSTTGAMQSYTWSLTYTVTTAAGSPAENSQQLPCMRNTHVPSSSVTHRIPVYPHREEHGYTGSYNYGSYGNQHPHPMQSQYPALPHDTAISGPLHYAPYHRSSAQYPFNSPTSRMEPCLMSSTPRLHPTPVTPRWPEVPSANTCYTSPSVHSARYGNSSDMYTPLTTRRNSEYEHMQHFPGFAYINGEASTGWAK

SEQ ID NO: 24 - protein, 928 aa, Homo Sapiens
MAKVPELEDTFLQAQPAPQLSPGIQEDCCVQLLGKGLLVYPEETVYLAAEGQPGGEQGGGEKGEDPELPGAVKSEMHLNNGNFSSEEEDADNHDSKTKAADQYLSQKKTITQIVKDKKKQTQLTLQWLEENYIVCEGVCLPRCILYAHYLDFCRKEKLEPACAATFGKTIRQKFPLLTTRRLGTRGHSKYHYYGIGIKESSAYYHSVYSGKGLTRFSGSKLKNEGGFTRKYSLSSKTGTLLPEFPSAQHLVYQGCISKDKVDTLIMMYKTHCQCILDNAINGNFEEIQHFLLHFWQGMPDHLLPLLENPVIIDIFCVCDSILYKVLTDVLIPATMQEMPESLLADIRNFAKNWEQWVVSSLENLPEALTDKKIPIVRRFVSSLKRQTSFLHLAQIARPALFDQHVVNSMVSDIERVDLNSIGSQALLTISGSTDTESGIYTEHDSITVFQELKDLLKKNATVEAFIEWLDTVVEQRVIKTSKQNGRSLKKRAQDFLLKWSFFGARVMHNLTLNNASSFGSFHLIRMLLDEYILLAMETQFNNDKEQELQNLLDKYMKNSDASKAAFTASPSSCFLANRNKGSMVSSDAVKNESHVETTYLPLPSSQPGGLGPALHQFPAGNTDNMPLTGQMELSQIAGHLMTPPISPAMASRGSVINQGPMAGRPPSVGPVLSAPSHCSTYPEPIYPTLPQANHDFYSTSSNYQTVFRAQPHSTSGLYPHHTEHGRCMAWTEQQLSRDFFSGSCAGSPYNSRPPSSYGPSLQAQDSHNMQFLNTGSFNFLSNTGAASCQGATLPPNSPNGYYGSNINYPESHRLGSMVNQHVSVISSIRSLPPYSDIHDPLNILDDSGRKQTSSFYTDTSSPVACRTPVLASSLQTPIPSSSSQCMYGTSNQYPAQETLDSHGTSSREMVSSLPPINTVFMGTAAGGT

SEQ ID NO: 25 - protein, 525 aa, Homo Sapiens
MLRTILDAPQRLLKEGRASRQLVLVVVFVALLLDNMLFTVVVPIVPTFLYDMEFKEVNSSLHLGHAGSSPHALASPAFSTIFSFFNNNTVAVEESVPSGIAWMNDTASTIPPPATEAISAHKNNCLQGTGFLEEEITRVGVLFASKAVMQLLVNPFVGPLTNRIGYHIPMFAGFVIMFLSTVMFAFSGTYTLLFVARTLQGIGSSFSSVAGLGMLASVYTDDHERGRAMGTALGGLALGLLVGAPFGSVMYEFVGKSAPFLILAFLALLDGALQLCILQPSKVSPESAKGTPLFMLLKDPYILVAAGSICFANMGVAILEPTLPIWMMQTMCSPKWQLGLAFLPASVSYLIGTNLFGVLANKMGRWLCSLIGMLVVGTSLLCVPLAHNIFGLIGPNAGLGLAIGMVDSSMMPIMGHLVDLRHTSVYGSVYAIADVAFCMGFAIGPSTGGAIVKAIGFPWLMVITGVINIVYAPLCYYLRSPPAKEEKLAILSQDCPMETRMYATQKPTKEFPLGEDSDEEPDHEE

SEQ ID NO: 26 - protein, 282 aa, Homo Sapiens
MSCGRPPPDVDGMITLKVDNLTYRTSPDSLRRVFEKYGRVGDVYIPREPHTKAPRGFAFVRFHDRRDAQDAEAAMDGAELDGRELRVQVARYGRRDLPRSRQGEPRGRSRGGGYGRRSRSYGRRSRSPRRRHRSRSRGPSCSRSRSRSRYRGSRYSRSPYSRSPYSRSRYSRSPYSRSRYRESRYGGSHYSSSGYSNSRYSRYHSSRSHSKSGSSTSSRSASTSKSSSARRSKSSSVSRSRSRSRSSSMTRSPPRVSKRKSKSRSRSKRPPKSPEEEGQMSS

SEQ ID NO: 27 - protein, 1488 aa, Homo Sapiens
MAEARKRRELLPLIYHHLLRAGYVRAAREVKEQSGQKCFLAQPVTLLDIYTHWQQTSELGRKRKAEEDAALQAKKTRVSDPISTSESSEEEEEAEAETAKATPRLASTNSSVLGADLPSSMKEKAKAETEKAGKTGNSMPHPATGKTVANLLSGKSPRKSAEPSANTTLVSETEEEGSVPAFGAAAKPGMVSAGQADSSSEDTSSSSDETDVEGKPSVKPAQVKASSVSTKESPARKAAPAPGKVGDVTPQVKGGALPPAKRAKKPEEESESSEEGSESEEEAPAGTRSQVKASEKILQVRAASAPAKGTPGKGATPAPPGKAGAVASQTKAGKPEEDSESSSEESSDSEEETPAAKALLQAKASGKTSQVGAASAPAKESPRKGAAPAPPGKTGPAVAKAQAGKREEDSQSSSEESDSEEEAPAQAKPSGKAPQVRAASAPAKESPRKGAAPAPPRKTGPAAAQVQVGKQEEDSRSSSEESDSDREALAAMNAAQVKPLGKSPQVKPASTMGMGPLGKGAGPVPPGKVGPATPSAQVGKWEEDSESSSEESSDSSDGEVPTAVAPAQEKSLGNILQAKPTSSPAKGPPQKAGPVAVQVKAEKPMDNSESSEESSDSADSEEAPAAMTAAQAKPALKIPQTKACPKKTNTTASAKVAPVRVGTQAPRKAGTATSPAGSSPAVAGGTQRPAEDSSSSEESDSEEEKTGLAVTVGQAKSVGKGLQVKAASVPVKGSLGQGTAPVLPGKTGPTVTQVKAEKQEDSESSEEESDSEEAAASPAQVKTSVKKTQAKANPAAARAPSAKGTISAPGKVVTAAAQAKQRSPSKVKPPVRNPQNSTVLARGPASVPSVGKAVATAAQAQTGPEEDSGSSEEESDSEEEAETLAQVKPSGKTHQIRAALAPAKESPRKGAAPTPPGKTGPSAAQAGKQDDSGSSSEESDSDGEAPAAVTSAQVIKPPLIFVDPNRSPAGPAATPAQAQAASTPRKARASESTARSSSSESEDEDVIPATQCLTPGIRTNVVTMPTAHPRIAPKASMAGASSSKESSRISDGKKQEGPATQVSKKNPASLPLTQAALKVLAQKASEAQPPVARTQPSSGVDSAVGTLPATSPQSTSVQAKGTNKLRKPKLPEVQQATKAPESSDDSEDSSDSSSGSEEDGEGPQGAKSAHTLGPTPSRTETLVEETAAESSEDDVVAPSQSLLSGYMTPGLTPANSQASKATPKLDSSPSVSSTLAAKDDPDGKQEAKPQQAAGMLSPKTGGKEAASGTTPQKSRKPKKGAGNPQASTLALQSNITQCLLGQPWPLNEAQVQASVVKVLTELLEQERKKVVDTTKESSRKGWESRKRKLSGDQPAARTPRSKKKKKLGAGEGGEASVSPEKTSTTSKGKAKRDKASGDVKEKKGKGSLGSQGAKDEPEEELQKGMGTVEGGDQSNPKSKKEKKKSDKRKKDKEKKEKKKKAKKASTKDSESPSQKKKKKKKKTAEQTV

SEQ ID NO: 28 - protein, 499 aa, Homo Sapiens
MLSLSPILLYTCEMFQDPVAFKDVAVNFTQEEWALLDISQKNLYREVMLETFWNLTSIGKKWKDQNIEYEYQNPRRNFRSVTEEKVNEIKEDSHCGETFTPVPDDRLNFQKKKASPEVKSCDSFVCEVGLGNSSSNMNIRGDTGHKACECQEYGPKPWKSQQPKKAFRYHPSLRTQERDHTGKKPYACKECGKNIIYHSSIQRHMVVHSGDGPYKCKFCGKAFHCLSLYLIHERTHTGEKPYECKQCGKSFSYSATHRIHERTHIGEKPYECQECGKAFHSPRSCHRHERSHMGEKAYQCKECGKAFMCPRYVRRHERTHSRKKLYECKQCGKALSSLTSFQTHIRMHSGERPYECKTCGKGFYSAKSFQRHEKTHSGEKPYKCKQCGKAFTRSGSFRYHERTHTGEKPYECKQCGKAFRSAPNLQLHGRTHTGEKPYQCKECGKAFRSASQLRIHRRIHTGEKPYECKKCGKAFRYVQNFRFHERTQTHKNALWRKTL

SEQ ID NO: 29 - protein, 426 aa, Homo Sapiens
MAGLGFWGHPAGPLLLLLLLVLPPRALPEGPLVFVALVFRHGDRAPLASYPMDPHKEVASTLWPRGLGQLTTEGVRQQLELGRFLRSRYEAFLSPEYRREEVYIRSTDFDRTLESAQANLAGLFPEAAPGSPEARWRPIPVHTVPVAEDKLLRFPMRSCPRYHELLREATEAAEYQEALEGWTGFLSRLENFTGLSLVGEPLRRAWKVLDTLMCQQAHGLPLPAWASPDVLRTLAQISALDIGAHVGPPRAAEKAQLTGGILLNAILANFSRVQRLGLPLKMVMYSAHDSTLLALQGALGLYDGHTPPYAACLGFEFRKHLGNPAKDGGNVTVSLFYRNDSAHLPLPLSLPGCPAPCPLGRFYQLTAPARPPAHGVSCHGPYEAAIPPAPVVPLLAGAVAVLVALSLGLGLLAWRPGCLRALGGPV

SEQ ID NO: 30 - protein, 522 aa, Homo Sapiens
MAVNKGLTLLDGDLPEQENVLQRVLQLPVVSGTCECFQKTYTSTKEAHPLVASVCNAYEKGVQSASSLAAWSMEPVVRRLSTQFTAANELACRGLDHLEEKIPALQYPPEKIASELKDTISTRLRSARNSISVPIASTSDKVLGAALAGCELAWGVARDTAEFAANTRAGRLASGGADLALGSIEKVVEYLLPPDKEESAPAPGHQQAQKSPKAKPSLLSRVGALTNTLSRYTVQTMARALEQGHTVAMWIPGVVPLSSLAQWGASVAMQAVSRRRSEVRVPWLHSLAAAQEEDHEDQTDTEGEDTEEEEELETEENKFSEVAALPGPRGLLGGVAHTLQKTLQTTISAVTWAPAAVLGMAGRVLHLTPAPAVSSTKGRAMSLSDALKGVTDNVVDTVVHYVPLPRLSLMEPESEFRDIDNPPAEVERREAERRASGAPSAGPEPAPRLAQPRRSLRSAQSPGAPPGPGLEDEVATPAAPRPGFPAVPREKPKRRVSDSFFRPSVMEPILGRTHYSQLRKKS

SEQ ID NO: 31 - protein, 48 aa, Homo Sapiens
PLPSSQPGGLGPALHQFPAGNTDNMPLTGQMELSQIAGHLMTPPISPA

SEQ ID NO: 32 - protein, 48 aa, Homo Sapiens
MVNQHVSVISSIRSLPPYSDIHDPLNILDDSGRKQTSSFYTDTSSPVA

SEQ ID NO: 33 - protein, 48 aa, Homo Sapiens
MPLTGQMELSQIAGHLMTPPISPAMASRGSVINQGPMAGRPPSVGPVL

SEQ ID NO: 34 - protein, 48 aa, Homo Sapiens
NRNKGSMVSSDAVKNESHVETTYLPLPSSQPGGLGPALHQFPAGNTDN

SEQ ID NO: 35 - protein, 48 aa, Homo Sapiens
AGSPYNSRPPSSYGPSLQAQDSHNMQFLNTGSFNFLSNTGAASCQGAT

SEQ ID NO: 36 - protein, 48 aa, Homo Sapiens
SAPSHCSTYPEPIYPTLPQANHDFYSTSSNYQTVFRAQPHSTSGLYPH

SEQ ID NO: 37 - protein, 48 aa, Homo Sapiens
TKAADQYLSQKKTITQIVKDKKKQTQLTLQWLEENYIVCEGVCLPRCI

SEQ ID NO: 38 - protein, 48 aa, Homo Sapiens
GSPVEVQEAGTQQSLQAANKSGTQRSPEAASKAVTQRFREDARDPVTR

SEQ ID NO: 39 - protein, 48 aa, Homo Sapiens
TQRSVEVREAGTQRSVEVQEVGTQGSPVEVQEAGTQQSLQAANKSGTQ

SEQ ID NO: 40 - protein, 48 aa, Homo Sapiens
ALAQDVATQKAETQRSSIEVREAGTQRSVEVREAGTQRSVEVQEVGTQ

SEQ ID NO: 41 - protein, 48 aa, Homo Sapiens
TGLSLVGEPLRRAWKVLDTLMCQQAHGLPLPAWASPDVLRTLAQISAL

SEQ ID NO: 42 - protein, 49 aa, Homo Sapiens
LQLPVVSGTCECFQKTYTSTKEAHPLVASVCNAYEKGVQSASSLAAWSM

SEQ ID NO: 43 - protein, 49 aa, Homo Sapiens
PLVASVCNAYEKGVQSASSLAAWSMEPVVRRLSTQFTAANELACRGLDH

SEQ ID NO: 44 - protein, 49 aa, Homo Sapiens
ASVAMQAVSRRRSEVRVPWLHSLAAAQEEDHEDQTDTEGEDTEEEEELE

SEQ ID NO: 45 - protein, 49 aa, Homo Sapiens
HLEEKIPALQYPPEKIASELKDTISTRLRSARNSISVPIASTSDKVLGA

SEQ ID NO: 46 - protein, 49 aa, Homo Sapiens
STRLRSARNSISVPIASTSDKVLGAALAGCELAWGVARDTAEFAANTRA